SEQUENCE LISTING (150 sequences)

SEQ ID NO: 1 - protein, 126 aa, Bacillus cereus
MACECSSTVLTCCSDNSSNFVQDKVCNPWSSAEASTFTVYANNVNQNIVGTGYLTYDVGPGVSPANQITVTVLDSGGGTIQTFLVNEGTSISFTFRRFNIIQITTPATPIGTYQGEFCITTRYLMA

SEQ ID NO: 2 - protein, 125 aa, Bacillus mycoides
MSCNCSVTALTCCPDDSSNYVQDKVCNPWSSIENLTFTVYGNNVNQNIVGTGYITYDVGPGASPANDITVTVLDSGGGPIQTFTMVEGTSIAFTFRRFNTIQITTPATAGTYQGEFCITTRYAMS

SEQ ID NO: 3 - protein, 126 aa, Bacillus cytotoxicus
MACNCSVTALTCCPDESNNFVQDKVCNPWSSAEASTFTVYGNNINQNIVGTGYLTYDVGPGVSPANAITVNVLDSGGGIIQTFTVTEGMSVAFTFRRFDVIQIVTPATPAGTYQGEFCITTRYVMS

SEQ ID NO: 4 - protein, 125 aa, Bacillus luti
MACECSSTVLTCCSDNSSNFVQDKVCNPWSSAEDSTFTVYANNVNQNIIGTGYLTYNVGPGVSPANQITVTVLDSGGGPIQTFLVNEGTSISFTFRRFNIIQITTPVTPVGTYQGEFCITTRYLM

SEQ ID NO: 5 - protein, 126 aa, Bacillus cereus
MACECSSTVLTCCSDNSSNFVQDKVCNPRSSAEASTFTVYANNVNQNIVGTGYLTYDVGPGVSPANQITVTVLDSGGGTIQTFLVNEGTSISFTFRRFNIIQITTPATPIGTYQGEFCITTRYLMA

SEQ ID NO: 6 - protein, 126 aa, Bacillus tropicus; identical to SEQ ID NO: 1

SEQ ID NO: 7 - protein, 126 aa, Bacillus wiedmannii
MACECSGTVLTCCSDNSSNFVQDKVCNSWSSAAASTFTVYANNVNQNIVGTGYLTYDVGPGVSPANQITVAVLDSGGGTIQSFTVSEGTSIAFTFRRFNIIQITTPATPLGTYQGEFCITTRYLIS

SEQ ID NO: 8 - protein, 117 aa, Bacillus cereus
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGTAAAETGEFCMTIRYTLS

SEQ ID NO: 9 - protein, 120 aa, Bacillus cereus
MGNCGTNLSCCANAQKTIVQDKVCIDWTITTIAAPGQIIYADNISQDIYASGYLKVETGTGPVTITFYSGGTGGTAVETIIVASGSSASFTVRRFDTVAILGTTLGETGEFCMTIRYSLS

SEQ ID NO: 10 - protein, 116 aa, Bacillus cytotoxicus
MKNYSANLSCCANSQKNIVQDKVCINWTATAAPAIIYADNIAQDIYASGYLKVDTGTGPVTLVFYSGGIGGTAVETITVATGSGASFTVRRFDTVTILGTAAAETGEFCMTIRYTL

SEQ ID NO: 11 - protein, 117 aa, Bacillus cereus; identical to SEQ ID NO: 8

SEQ ID NO: 12 - protein, 117 aa, Bacillus wiedmannii
MGNCSTNLSCCANSQKTIVQDKVCIDWTASATAEVIYADNISQDIYASGYLKVDTGTGPVTLVFYSGGIAGTALETIVVATGSSASFTVRRFDTVTILGTAAAETGEFCMTIRYSLS

SEQ ID NO: 13 - protein, 117 aa, Bacillus luti
MGNCSTNLSCCANGQKTIVQDKVCIDWTAAVTAAIIYADNISQDIYASGYLKVDTGTGPVTINFYSGGTGGTIVETITVASGSSASFTVRRFDTVTIIGTAAAETGEFCMTIRYTLS

SEQ ID NO: 14 - protein, 117 aa, Bacillus tropicus
MGNCSTNLSCCANGQKTIVQDKVCIDWTAAATAAIIYADNISQDIYASGYLKVDTGTGPVTIVFYSGGVTGTALETIVVATGSSASFTVRRFDTVTILGTAAGETGEFCMTIRYTLS

SEQ ID NO: 15 - protein, 155 aa, Bacillus cereus
MKPHKNIGCFAPLSIICQPTCPCPPPILPPERGDAELVTNEFAGDILISNDFIPISQKQLKQTNTTVNIWKNDGIVSLSGTISIYNNRNSTNALSIQIISSTTNTFTALPGNTISYTGFDLQSVSVIDIPSDPSIYIEGRYCFQLTYCKSKRDCL

SEQ ID NO: 16 - protein, 150 aa, Bacillus cytotoxicus
MQKHKKIGCFAPLSIICEVPCPPPPPISNAEFVTNEFAGNFLITNDTFLPETNQLKQSDSTFELWKGDGLSSLSGSISVYNNRNSTTAITVQIVGETTNTLTALPGNTVSYTGSGLQSVSLINIPSDSSVYIEGRYCCQITYCKFKSDCM

SEQ ID NO: 17 - protein, 155 aa, Bacillus cereus
LKPHKNIGCFAPLSIICQPTCPCPPPISPPERGDAELVTNEFAGDILISNDFIPISQKQLKQTNTTVNIWKNDGIISLSGTISIYNNRNSTNALSIQIISSTTNTFTVLPGNTISYTGFDLQSVSVIDIPSDPSIYIEGRYCFQLTYCKSKRDCL

SEQ ID NO: 18 - protein, 155 aa, Bacillus wiedmannii
LKPHKNIGCFAPLSIICQPICPCPPPIPPPERCDAELITNEFAGDILISNDFIPISQKQLKQTYSTVTIWKNGGIVSLSGTISIYNNRNSTSALSVQIIGSTTNIFTVLPGNTISYTGFDLLSVSIIDIPSDPSIYIEGRYCFQLTYCKSKLDCL

SEQ ID NO: 19 - protein, 155 aa, Bacillus wiedmannii; identical to SEQ ID NO: 18

SEQ ID NO: 20 - protein, 155 aa, Bacillus luti
LKPHKNIGCFAPLSIICQPICPCPPPIPPPEQCDAELITNEFAGNIFISNDFISVAQRQSKQIYPTLTIWKDDGSISLSGTISVYNSKNSTDALTVQIVSNNTNTFTVLPGNTISYTGSNLQSVSIIDIPTDPSTYIEGRYCFQLTYCKSKRDCL

SEQ ID NO: 21 - protein, 126 aa, Bacillus cytotoxicus
MACNCSVTALTCCPDESNNFVQDKVCNPWSSSEASTFTVYGNNINQNIVGTGYLTYDVGPGVSPANAITVNVLDSGGGIIQTFTVTEGMSVAFTFRRFDVIQIVTPATPVGTYQGEFCITTRYVMS

SEQ ID NO: 22 - protein, 122 aa, Bacillus wiedmannii
MSCECSGIALTCCPDKNYVQDKVCSPWSATVVATAIDNVLYTNNINQNVVGTGFVKYDVGPGPITVEVLDSAGTVLDTQTLNPGTSIGFTYRRFDIIQVVLPATPAGTYQGEFCITTRYPLS

SEQ ID NO: 23 - protein, 119 aa, Bacillus manliponensis
MSCECTSTVLSCCPDKTFVQDKVCSPWSGTVVAADVTVILYTNNINQTVVGTGFIKYDVGPTDITVQVLDSTATIIDTQTLSPGSSLGFTYRQFNTIQVILPLATPGVYQGEFCITSRY

SEQ ID NO: 24 - protein, 122 aa, Bacillus cereus
MSCECSGAALTCCPDKNYVQDKVCSPWSGTVVATAITNVLYNNNINQNVIGTGFVRYDVGPAAITLTVLDSAGNTLDTQTLNPGTSIAFTYRRFETIEVTLPAATAGTYQGEFCITTRYPLS

SEQ ID NO: 25 - protein, 122 aa, Bacillus cereus
MSCECSGSALTCCPDKNYVQDKVCSPWSGTVVATAITNVLYNNNINQNMIGTGFVRYDVGPAPITLTVLDAAGATIDTQTLNPGTSIAFTYRRFVTIEVTLPAATAGTYQGEFCITTRYPLS

SEQ ID NO: 26 - protein, 122 aa, Bacillus anthracis
MSCECSGTALTCCPDKNYVQDKVCSPWSATVVATAIDNVLYTNNINQNVVGTGFVKYDVGPGPITVEALDSAGTVIDTQTLNPGTSIGFTYRRFDIIQVVLPATPAGTYQGEFCITTRYPLS

SEQ ID NO: 27 - protein, 122 aa, Bacillus toyonensis
MSCECSGSALTCCPDKNYVQDKVCSPWSATVVVTAIDNVLYTNNINQNVIGTGFVKYDVGPGPITVEALDSAGATIDTQTLNPGTSIAFTYRRFDSIQVVLPATPAGTYQGEFCITTRYPLS

SEQ ID NO: 28 - protein, 122 aa, Bacillus mycoides
MSCECSGSALTCCPDKNYVQDKVCSPWSATVVATAIDNVLYTNNINQNVIGTGFVKYDVGPGPITVEALDSAGATIDTQTLNPGTSIAFTYRRFDSIQVVLPATPAGTYQGEFCITTRYSLS

SEQ ID NO: 29 - protein, 117 aa, Bacillus cytotoxicus
MENYSANLSCCANSQKNIVQDKVCINWTASAAPAIIYADNIAQDIYASGYLKVDTGTGPVTLVFYSGGIGGTAVETITVATGSGASFTVRRFDTVTILGTASAETGEFCMTIRYTLI

SEQ ID NO: 30 - protein, 113 aa, Bacillus wiedmannii
LTCCPDKNYVQDKVCSPWSATVVATAIDNVLYTNNINQNVVGTGFVKYDVGPGPITVEVLDSAGTVLDTQTLNPGTSIGFTYRRFDIIQVVLPATPAGTYQGEFCITTRYPLS

SEQ ID NO: 31 - protein, 114 aa, Bacillus manliponensis
MGSNYSGLSCCSNKTIVQDKVCLDWSIDGTGAVVVYTNNVTQEIVASGYVKYDVGVGDITVEFLVGTTPVETLTISPGSSAAFTVRRFTDIEITTAGVGEHQGEFCITLRYPIS

SEQ ID NO: 32 - protein, 112 aa, Bacillus cereus
MGNSSLSCCSNNTLVQDQVCIDWSATGAVTETVYTNNITQDLYASGYVKYDVGTAPITVNFLVGATVVNTITVQPQSSGSFTVRYFTTIQIVTTGTGVSQGQLCLTVRYPIS

SEQ ID NO: 33 - protein, 110 aa, Bacillus thuringiensis
NSNLSCCSSKTLVQDQVCIDWAATGAVTETVYTNNITQDLYASGYVKYDVGTAPITVNFLVGATVVNTITVQPQSSGSFTVRYFTTIQIVTTGTGVSQGQLCLTVRYPIS

SEQ ID NO: 34 - protein, 111 aa, Bacillus mycoides
CNSNLSCCSNKTLVQDQVCIDWSATGAVTETVYTNNITQDLYASGYVKYDVGVNPVTVNFLVGATVVNTITIQPQSSGSFTVRYFTTIQIVTTGIGTSQGQLCLTVRYPIS

SEQ ID NO: 35 - protein, 114 aa, Bacillus anthracis
MGMRSSSLSCCSNKTLVQDQVCTDWSITGAGTQIVYTNNIAQEVYGSGYVKYDVGTAPITVDFLVGATVVDTITVQPQSSGTFTVRYFTTVRITTTGTTVNQGEFCITVRYPIS

SEQ ID NO: 36 - protein, 114 aa, Bacillus toyonensis
MGMRSSSLSCCSNKTLVQDQVCTDWSITGAGTQIVYTNNITQEVYGSGYVKYDVGANPITVDFLVGATVVDTITVQPQSSGTFTIRYFTTIRITTTGTTVSQGEFCITVRYPIS

SEQ ID NO: 37 - protein, 114 aa, Bacillus mycoides
MGMRSSSLSCCSNKTLVQDQVCTDWSITGAGNQIVYTNNLTQEIYGSGYVKYDTGANPITVDFLVGATVVDTITVQPQSSGTFTVRHFTTVRITTTGTTVNQGEFCITVRYSIS

SEQ ID NO: 38 - protein, 150 aa, Bacillus cytotoxicus
MQKHKKIGCFAPLSIICEVPCPPPPPISNAEFVTNEFVGNFLITNDTFLPETNQLKQSDSTFELWKGDGLSSLSGSISVYNNRNSTTAITVQIVGETTNTLTALPGNTVSYTGSGLQSVSLINIPSDPSVYIEGRYCCQITYCKFKSDCM

SEQ ID NO: 39 - protein, 135 aa, Bacillus thuringiensis
KKNIGCYAPLSIICPPYDPPNPPEPPGCELITNEFAGNFLITKEIPPPSENPTLILWEGDGVVKISGTISVYNSTSSTAGITVQIIGKVTNTFTVYPGNTMSYTGQSLQSVAIIDIPNNPTRYMEGKYCCQFSFC

SEQ ID NO: 40 - protein, 135 aa, Bacillus mycoides
KKNIGCYAPLSIICPPYDPPKPPKPPSCELVTNEFAGNFFITKEIPPPSENSTLVLWEGDGLLKISGTISVYNSTSSTGGITVQIIGQVTNTFTVYPGNTMSYTGQSLQSVTIVNIPNNPIQYIEGKYCCQFSFC

SEQ ID NO: 41 - protein, 135 aa, Bacillus wiedmannii
KKNIGCYAPLSIICPPYDPPNPPNPSSCELVNNEFAGNFFITKEIPPPSESSTLILWEGDGVLKISGTISVYNSTSSTEAVTVQIIGQVTNTFTVYPGNTMSYTGQSLQSLAIIDIPNNPTRYMEGKYCCQFSFC

SEQ ID NO: 42 - protein, 135 aa, Bacillus pacificus
KKNIGCYAPLSIICPPYDPPTPPDPPSCELVNNEFAGNFFITKEIPPPSEGSTLILWEGDGVLKISGNISVYNSTSSTEAVTVQIIGQVTNTFTVYPGNTMSYTGQSLQSVAIINLPNNPIRYIEGKYCCQFSFC

SEQ ID NO: 43 - protein, 134 aa, Bacillus thuringiensis
KNIGCYAPLSIICPPYDPPDPPNPPSCELVTNEFAGNFFITKEIPPPSKSPTLTLWEGDGILKISGTISVYNSTSSTEAVTVQIIGQVTNAFTVYPGNTMSYTGQSLQSMTIINIPHNPTRYIEGKYCCQFSFC

SEQ ID NO: 44 - protein, 143 aa, Bacillus mycoides
KKNIGCYAPLSIICPPYPPPNPPEPPGCELVMNEFAGNFFITNEIPLPPKENPNPFRTLPLWEGDGVLKISGTISIYNSTSSTGEVTVQIVGQVTSTFIFYPGNTMSYTGKSLQSVTIINIPNTPTQYIEGKYCCQFSFCLNR

SEQ ID NO: 45 - protein, 136 aa, Bacillus anthracis
KNIGCFAPLSIICPDPCPSTPPPNPNCERVTNEFAGNFLITKNTMPSAKGASQSMILWQSDGILLISGTVSVYNSTSSTEAITIEIVGAVTNIFTMFPGNTISYTGKDLQSVSIANIQHNPSLYLEGKYCCQFTCC

SEQ ID NO: 46 - protein, 136 aa, Bacillus wiedmannii
KNIGCFAPLSIICPDPCPPPPPPNPSCERVTNEFAGNFLITNNTISSVKDTSQSMILWQSDGILLISGTVSVYNSTSSTEAITIEIVGAVTNIFTVFPGNTISYTGKNLQSVSITNIQNNPSLYLEGKYCCQFTCC

SEQ ID NO: 47 - protein, 145 aa, Bacillus manliponensis
LSNRKRIGCFAPLGMNCYPPSPPLFPPPKPIETELITNEFCGNFLVTDQPLPSRETLELWQRDETSLITGTVSIYNSSNSTHPVSIQIIDKIAHQFIVFPGNTTSFTGNALQSISIIDIPNDSPLYIEGKYCCQFTYCRKKMNCM

SEQ ID NO: 48 - protein, 140 aa, Bacillus mycoides
LNKKKQIGCYAPLELSCHPIYPPCPPQPNPGCELVSNEFSGNFLVRDGQVQTLQLWKSDGITSISGTISVYNSSNSTDPATIVISGISTTTLIVLPGNTSSFTGTDLQSVEMIDIPNTSLSYLEGKYCCQFTYCHSKSNA

SEQ ID NO: 49 - protein, 113 aa, Bacillus cereus
MAQIGNCCTEQLCCVNDAVCCTIILDDTGGTALPIWDDATTFVINGTIMVENNGTVGVGPTAALTVNGTAVGGFVVAPGERSITMNDINSIAIVGAGTGTSSVKISFSINYKF

SEQ ID NO: 50 - protein, 112 aa, Bacillus sp. ms-22
MLGGCSNEQAGFVNDAVCFTLDLAAAADAVVVWTDGTPYVINGTIMGENNGIVAVAPTVTLEINGTAVPDFTVPPGESRSITLSDLNSIGIVATGGTSTGNIVVSFSLNYQY

SEQ ID NO: 51 - protein, 114 aa, Bacillus sp. LLTC93
MAILGGCSNEQTGFVNDAVCFTVDLAAAADAVVVWTDGTPFVINGTIMVENNGIVAVAPTVTLEVNGTAVPDFTVPPGESRSITLNDINSIGIVGTGGTSTGNIVVSFSLNYQY

SEQ ID NO: 52 - protein, 115 aa, Bacillus pumilus
MAILGGCSNEQTGFVNDAVCFTVDLGATAPDAVVVWTDGTPFVINGTIMVENNGIAAVAPTVSLQVNDAAVPGFTVPPGESRSITLNDINSIGIIGTGGTSTGNIVVSFSLNYQY

SEQ ID NO: 53 - protein, 115 aa, Bacillus atrophaeus
MAMLGGCSNEQMGFVNDAVCFTIDLGDTGAVATPVWEDGTSFVINGTIMVENNGIVGVGPTASLTVNGTAVAGFTVAPGESRSITLNDINSIGIVGTGGTGTANVTVSFSLNYQY

SEQ ID NO: 54 - protein, 116 aa, Peribacillus psychrosaccharolyticus
MAQIGSCNGEQLVGVNDSVCLTLELDDAVAPTVVWTDVTPYVINGTIMIENNGIVGVGQDAALSVNGTAIADFVVVPGEARSITLDNINSISLLGSGGEAGASSSVNVAFSLNYKF

SEQ ID NO: 55 - protein, 115 aa, Amphibacillus marinus
MAQLGSCCGDELVGVNDAVCFTVDLEATGEGADLILWEDETSFIINGSIMVENNGIIGASPTAALTVNGAAIPGFTVAPGEARSITLNDIETIGLTGTGTGTGSVKVSFSINYKY

SEQ ID NO: 56 - protein, 115 aa, Bacillus cereus
MALLGGCSDMSVSSVNDAVCFTITLEDTTAGTPVEVWEDSTGFAINGTIMIENNGITETSPTASLLVNDTEVTGFTVAPGESRAITLDNLNSIGISGAGTGSSAVKVSFSLNYKF

SEQ ID NO: 57 - protein, 116 aa, Bacillus endophyticus
MAQLGGCSSDELGVVNDAVCVTIDLPVTTVGTPIPVWTDTSTFAINGTIVVENNGTVGISPTASLEVNGTAVTDFTVGPGEAQSITLNNIESIAIAGTGGTGTAIVKVAFSLNYKF

SEQ ID NO: 58 - protein, 114 aa, Peribacillus loiseleuriae
MAQIGNCCSEQLVGVNDALCFTIDLADTATDTIDLWNNNTTFVINGTIMVENNGINGVTTTATLMVNGTAVTGFTVGPGEAMSVTMNNINSIGLVGAGTGTASVKVSFSLNYKF

SEQ ID NO: 59 - protein, 118 aa, Bacillus cereus
MAQIGNCCNDQLVGVNDALCFTIVLDDTNGTAIDLWNDATTFVINGTIMVENNGTIGVAPTASLTVNGTPVGGFIVGPGEARSITMNDINSIGIVGAGGTPVGSTANVKVSFSLNYKF

SEQ ID NO: 60 - protein, 118 aa, Peribacillus loiseleuriae
MAQIGNCCNDSLVGVNDALCFTIDIADTDGTPLVLWNDATTFVINGTIMVENNGTIGVSPTAALTVNGTPIAGFIVGPGEAKSVTLNDINSIGIIGTGGTPAGSTASVKVSFSLNYKF

SEQ ID NO: 61 - protein, 118 aa, Bacillus megaterium
MAQLGSCCSDSLGVVNDAVCFTVDLADTGGTEIVLWNDSTTFIINGTVMVENNGVTGVGPTASLTVNGVAVAGFVVEPGSARAITVNDINTIGIIGTGGAPLGSTSNVKISFSLNYKF

SEQ ID NO: 62 - protein, 118 aa, Bacillus aryabhattai
MADLGSCCSDNLGVVNDAVCFTISLGDTDGSPISIWSNGTTFVVNGTIMVENNGLANVGSTASLFVNGSAIEGFVVAPGEARAITINDINIIGIVGAGGTPVGSTSKVKISFSINYKF

SEQ ID NO: 63 - protein, 114 aa, Lysinibacillus sinduriensis
MAQLGGCNGEQLVGVNDAICFTVLLDDTGATPLPIWSDATTYIINGTIMVENNGVVGASPTAALTVNGTAVDGFVVAPGEARSITMNDINSIALTGTGAGTANVKVSFSLNYKF

SEQ ID NO: 64 - protein, 115 aa, Bacillus megaterium
MAQLGGCSSEGDFGSVNDAVCTTVELSDTGVTPITIWADTTSYIINGTILIENNGIIGASPTASLLVNGTPVADFTVAAGEARSLTVDNINLIQLAGVGAGTARVKVSFSLNYQF

SEQ ID NO: 65 - protein, 114 aa, Bacillus sp. YR335
MSKLGNCCQDQLVGVNDALCFTINLTNTGGSPIPIWDDATSFNINGTILIENKGVIGASPTAALVVNGTPVAGFIVGAGEVRSITMSNINSIRITGAGTGTAPVMISFSINYKY

SEQ ID NO: 66 - protein, 113 aa, Vibrio vulnificus
MQYGNCCNGLFGIVSDAVCFTVNLSNTAGVALELWNDATPFIINGTIVVENNGIIGASPTAALSVNGTPVGGFIVTAGQVKSITMNNINSIGLIGSGTGTASVRVSFSLNYKF

SEQ ID NO: 67 - protein, 114 aa, Peribacillus asahii
MANLGGFSNNPLNVISDSVSCTIESGDTNGAATTIWQDDTPFVINGTIVVDNKGKVGASPKAALLVNGTAINGFVVAPGECRSITVNDLHSISIIVTGTGTAKINISFSLNYKF

SEQ ID NO: 68 - protein, 116 aa, Bacillus cereus
MAQLGNCSNSQQNTVNDTVCNNIVLEDTAGVPISIWDNNTNMIINGTILVQNNRIIGIGTTTALTVNDTTVCGFVVRPGESRSITMNDINSIGIVGVGAGVNSSNVKISFSINYKF

SEQ ID NO: 69 - protein, 114 aa, Bacillus cereus
MTQLGNYSSSQQSTVNDAICSDIVLEDTAGVPVPIWDDNTNMIINGTILVQNNGIIGVGATAALIVNGTPVGGFIVGPGESRSITINDINSIGIVGAGTNSSNIKVSFSINYKF

SEQ ID NO: 70 - protein, 114 aa, Bacillus cereus group
MAQLGNCSNFQQQAVNDAVCCNIVLSDTGGTPVPIWNDDTSRIINGTILVQNDGIIGVGATASLIVNGTAVGGFTVGPGESRSITMSDINSIGIVGAGTNSSNVKVSFSINYKF

SEQ ID NO: 71 - protein, 114 aa, Halobacillus
MAQLGSCISNSYCCVNDAVCCTISLEDTGEIPVTVWEDVTDFVINGTIMIENNGITDTSPTAALYVNGEAIGDFVLAPGECRSITLNDINSIGIVGAGTGSSNVKVSFSINYKF

SEQ ID NO: 72 - protein, 114 aa, Virgibacillus dokdonensis
MAQLGLCNTDSLCCINDAVCCTITLEDTATTPISVWENASTFIINGTIVIENNGLVVGSPTAELYVNGTAVSGFVVEPGECRSITLEGVNSIGIVGSGTGSSNVKISFSINYKF

SEQ ID NO: 73 - protein, 114 aa, Bacillus sp. BPN334
MAQIGNCCTEQLCCVNDAVCCTIILDDTDGTALPIWDDSTTFVINGTIMVENNGAVGVGSTAALTVNGTAVGGFVVAPGECRSITMNDINSIAIVGSGTGTSSVKISFSLNYKF

SEQ ID NO: 74 - protein, 115 aa, Bacillus cereus ATCC 10987
MLAQIGNCCTEQLCCVNDAVCCTIILDDTGGTALPIWDDATTFVINGTIMVENNGTVGVGPTAALTVNGTAVGGFVVAPGECRSITMNDINSIAIVGAGTGTSSVKISFSINYKF

SEQ ID NO: 75 - protein, 114 aa, Paenibacillus sp. GM1FR
MAQIGNCNNTVSPAVVNDAVCTTISLSDTGGTPTIIYENSSTFLINGTILIENNGVGVSPTAALLIDGVQIPGFVVAPGSSRSYTAPTINSISLIGAGLGTTSIKIAFSLNYRF

SEQ ID NO: 76 - protein, 115 aa, Bacillus aurantiacus
MAQLGSCGRQQQDCCVNDAVCFNIEIETGGEEQLVWTNNGAFTINGTIVIENDGIEEGPSASLVVNGVATGVVVAPGESEAVTLDNLNSISVAPEGGGEGLTSDVKVAFSINYRF

SEQ ID NO: 77 - protein, 113 aa, Rummeliibacillus pycnus
MAKLGNCHMEPCCVNDAVCSTVHVVNQSLVTVWTNESPFNINGTIVVENPLQDGNATQQTVRLIITGSSGTDVNIAPGQTVALTVRGLHIIQVQGLTTGTTDVKVSFSLNYKF

SEQ ID NO: 78 - protein, 109 aa, Anaerobacillus alkalidiazotrophicus
MAQLGKCFPTTLCCVNDAVCCDISVTVGTGPVDLYTNSTNFPVNGTFMVDNSNSSTGNVTLTDGTNAIDVAPGTCQAITYSDINNGESITWQTTAAADFKVSFSINYKF

SEQ ID NO: 79 - protein, 111 aa, Lysinibacillus meyeri
MAKLGSCCGSKTSVHDSVCQDVTLTDETPVIVWENSTPFSINGTILVENASSSTADVTLTVTSNPATPAIAAIAPGNSVSVTANNISDIQLAGEAAEVANVKISYSLNYNF

SEQ ID NO: 80 - protein, 111 aa, Metasolibacillus fluoroglycofenilyticus
MAKLGSCCGSKTSVHDSVCQDLTLTDETAVTVWENSTPFAINGTILVENASSSTGDVTLTVTSSPATPAIPPITPGNSISITANNIADIQLAGTAASVANVKISYSLNYNF

SEQ ID NO: 81 - DNA, 429 bp, Artificial Sequence
  recombinant Ena1A nucleotide sequence
atgcatcaccatcaccatcacagcagcggtgaaaatctgtattttcagggcgcttgtgaatgtagcagcacagtcctgacctgttgttcggacaactctagtaattttgtgcaggataaagtttgcaacccctggtcttccgcggaagcaagcactttcaccgtatatgcgaacaatgttaaccaaaacattgtcggcaccggctatttaacatatgatgtggggcccggtgtgagcccggccaaccagattacggttacggttctggactccggcggcggtactatccagacctttctggtcaacgaagggacgtctatctcatttacctttcgccgctttaacattattcagattacaaccccagccacaccgattggcacgtatcagggtgaattttgcatcacgacccggtatttaatggcctaa

SEQ ID NO: 82 - protein, 142 aa, Artificial Sequence
  recombinant Ena1A amino acid sequence (with N-terminal 6xHis tag and TEV cleavage site)
MHHHHHHSSGENLYFQGACECSSTVLTCCSDNSSNFVQDKVCNPWSSAEASTFTVYANNVNQNIVGTGYLTYDVGPGVSPANQITVTVLDSGGGTIQTFLVNEGTSISFTFRRFNIIQITTPATPIGTYQGEFCITTRYLMA

SEQ ID NO: 83 - DNA, 399 bp, Artificial Sequence
  recombinant Ena1B nucleotide sequence
atgcaccaccaccaccatcattctagcggtgaaaacctgtactttcagggtaactgcagcaccaatctgtcatgctgtgccaatggtcagaagaccattgtccaggataaagtctgcatcgactggaccgcagccgctactgcagcaatcatttacgctgataatatcagccaagacatctacgcttcaggctatctgaaagtggatacaggtacgggtcccgtgaccatcgtcttttactctggtggagtcacaggcaccgctgtggagaccattgtggtcgccacgggttcgtcggccagctttacggtgcgccgttttgataccgtcactattctgggcaccgcagcagcggagactggtgagttttgtatgaccatccgttacactttgagctaa

SEQ ID NO: 84 - protein, 132 aa, Artificial Sequence
  recombinant Ena1B amino acid sequence (with N-terminal 6xHis tag and TEV cleavage site)
MHHHHHHSSGENLYFQGNCSTNLSCCANGQKTIVQDKVCIDWTAAATAAIIYADNISQDIYASGYLKVDTGTGPVTIVFYSGGVTGTAVETIVVATGSSASFTVRRFDTVTILGTAAAETGEFCMTIRYTLS

SEQ ID NO: 85 - DNA, 516 bp, Artificial Sequence
  recombinant Ena1C nucleotide sequence
atgcatcatcatcatcaccattccagcggtgagaatctctatttccaaggcaaaccgcacaaaaatatcggctgctttgcgccgctctccattatctgccagccgacctgtccgtgcccgccgccaattttaccgccggaacgcggtgacgccgagctggtcacaaatgaatttgcgggggacatcctgattagcaacgattttattccaattagccagaaacagctgaaacaaacaaacaccaccgttaatatctggaaaaacgacggaatcgtttccctgagcggcacgatttcaatttataataatcgcaattcgaccaacgcgctgtcgattcagattatcagtagtacgaccaatacctttacagcgctcccggggaatacgatttcctatactggttttgacctgcagtccgtctctgttatcgacattccaagcgatccaagtatctacattgagggccgctattgttttcagttaacttactgtaaatctaaacgcgattgtctttaa

SEQ ID NO: 86 - protein, 171 aa, Artificial Sequence [sequence omitted]

SEQ ID NO: 87 - protein, 116 aa, Artificial Sequence
  Ena1B_NM_Oslo
MGNCSTNLSCCANGQIIVQDKVCIDWTAAATAAIIYADNISQDIYASGYLKVDTGTGPVTIVFYSGGVTGTAVETIVVATGSSASFTVRRFDTVTILGTAAAETGEFCMTIRYTLS

SEQ ID NO: 88 - protein, 7 aa, Artificial Sequence
  synthetic peptide in figure 8
FCMTIRY

SEQ ID NO: 89 - protein, 7 aa, Artificial Sequence
  TEV cleavage site
ENLYFQG

SEQ ID NO: 90 - DNA, 20 bp, Artificial Sequence
  primer
aatggcgccagttcaattac

SEQ ID NO: 91 - DNA, 30 bp, Artificial Sequence
  primer
cctctctacatagcctttcccctctctctt

SEQ ID NO: 92 - DNA, 28 bp, Artificial Sequence
  Primer
aaggctatgtagagaggggaattagtat

SEQ ID NO: 93 - DNA, 22 bp, Artificial Sequence
  Primer
cctcctattctcccacctgaaa

SEQ ID NO: 94 - DNA, 20 bp, Artificial Sequence
  primer
tccatgtggtatggcaaaaa

SEQ ID NO: 95 - DNA, 28 bp, Artificial Sequence
  primer
ccatatattacatactaattcccctctc

SEQ ID NO: 96 - DNA, 33 bp, Artificial Sequence
  primer
aattagtatgtaatatatggtgatttaaagatt

SEQ ID NO: 97 - DNA, 20 bp, Artificial Sequence
  primer
aacctacttgcccctgtcct

SEQ ID NO: 98 - DNA, 20 bp, Artificial Sequence
  primer
cgcatcttgtttaggtgcaa

SEQ ID NO: 99 - DNA, 34 bp, Artificial Sequence
  primer
atttttttgttatccttttcataagactgtttac

SEQ ID NO: 100 - DNA, 32 bp, Artificial Sequence
  primer
tgaaaaggataacaaaaaaattattgcttttg

SEQ ID NO: 101 - DNA, 21 bp, Artificial Sequence
  primer
aggtggagggacaatccaaac

SEQ ID NO: 102 - DNA, 20 bp, Artificial Sequence
  primer
tccatgtggtatggcaaaaa

SEQ ID NO: 103 - DNA, 28 bp, Artificial Sequence
  primer
ccatatattacatagcctttcccctctc

SEQ ID NO: 104 - DNA, 32 bp, Artificial Sequence
  primer
aaaggctatgtaatatatggtgatttaaagat

SEQ ID NO: 105 - DNA, 20 bp, Artificial Sequence
  primer
aacctacttgcccctgtcct

SEQ ID NO: 106 - DNA, 24 bp, Artificial Sequence
  primer
aagtgcgtctaatcaacaaggaaa

SEQ ID NO: 107 - DNA, 21 bp, Artificial Sequence
  primer
gggaaatctcccatgaacaca

SEQ ID NO: 108 - DNA, 21 bp, Artificial Sequence
  primer
aggtggagggacaatccaaac

SEQ ID NO: 109 - DNA, 21 bp, Artificial Sequence
  primer
ggcgaaacgtaaatgaaatgc

SEQ ID NO: 110 - DNA, 21 bp, Artificial Sequence
  primer
ccactggaagtagcgcatctt

SEQ ID NO: 111 - DNA, 20 bp, Artificial Sequence
  primer
gccgctgttccaagaattgt

SEQ ID NO: 112 - DNA, 22 bp, Artificial Sequence
  primer
cctcctattctcccacctgaaa

SEQ ID NO: 113 - DNA, 23 bp, Artificial Sequence
  primer
ctccagcgaactcattggtaact

SEQ ID NO: 114 - DNA, 24 bp, Artificial Sequence
  primer
gggtgtacgagggtgatatgaatt

SEQ ID NO: 115 - DNA, 19 bp, Artificial Sequence
  primer
tgtcgttccgccaagtgtt

SEQ ID NO: 116 - DNA, 18 bp, Artificial Sequence
  primer
gcggatgttgttggacaa

SEQ ID NO: 117 - protein, 20 aa, Artificial Sequence
  primer
ACGTGCAAACACATGAATCG

SEQ ID NO: 118 - protein, 15 aa, Artificial Sequence
  N-terminal motif
  MISC_FEATURE (1)..(1): X can be Leu, Ile, Val, or Phe
  misc_feature (2)..(2): Xaa can be any naturally occurring amino acid
  misc_feature (5)..(14): Xaa can be any naturally occurring amino acid
XXCCXXXXXXXXXXC

SEQ ID NO: 119 - protein, 16 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(15): Xaa can be any naturally occurring amino acid
XXCCXXXXXXXXXXXC

SEQ ID NO: 120 - protein, 17 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(16): Xaa can be any naturally occurring amino acid
XXCCXXXXXXXXXXXXC

SEQ ID NO: 121 - protein, 16 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(15): Xaa can be any naturally occurring amino acid
XXXCCXXXXXXXXXXC

SEQ ID NO: 122 - protein, 17 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(16): Xaa can be any naturally occurring amino acid
XXXCCXXXXXXXXXXXC

SEQ ID NO: 123 - protein, 18 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(17): Xaa can be any naturally occurring amino acid
XXXCCXXXXXXXXXXXXC

SEQ ID NO: 124 - protein, 9 aa, Artificial Sequence
  C-terminal motif
  misc_feature (2)..(3): Xaa can be any naturally occurring amino acid
  misc_feature (5)..(8): Xaa can be any naturally occurring amino acid
GXXCXXXXY

SEQ ID NO: 125 - protein, 10 aa, Artificial Sequence
  C-termninal motif
  misc_feature (2)..(4): Xaa can be any naturally occurring amino acid
  misc_feature (6)..(9): Xaa can be any naturally occurring amino acid
GXXXCXXXXY

SEQ ID NO: 126 - protein, 18 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(17): Xaa can be any naturally occurring amino acid
XXCCXXXXXXXXXXXXXC

SEQ ID NO: 127 - protein, 19 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(18): Xaa can be any naturally occurring amino acid
XXCCXXXXXXXXXXXXXXC

SEQ ID NO: 128 - protein, 20 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(19): Xaa can be any naturally occurring amino acid
XXCCXXXXXXXXXXXXXXXC

SEQ ID NO: 129 - protein, 21 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(20): Xaa can be any naturally occurring amino acid
XXCCXXXXXXXXXXXXXXXXC

SEQ ID NO: 130 - protein, 12 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(11): Xaa can be any naturally occurring amino acid
XXCCXXXXXXXC

SEQ ID NO: 131 - protein, 13 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(12): Xaa can be any naturally occurring amino acid
XXCCXXXXXXXXC

SEQ ID NO: 132 - protein, 14 aa, Artificial Sequence
  N-terminal motif
  MISC_FEATURE (1)..(1): X is Leu, Ile, Val, or Phe
  misc_feature (2)..(2): Xaa can be any naturally occurring amino acid
  misc_feature (5)..(13): Xaa can be any naturally occurring amino acid
XXCCXXXXXXXXXC

SEQ ID NO: 133 - protein, 19 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(18): Xaa can be any naturally occurring amino acid
XXXCCXXXXXXXXXXXXXC

SEQ ID NO: 134 - protein, 20 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(19): Xaa can be any naturally occurring amino acid
XXXCCXXXXXXXXXXXXXXC

SEQ ID NO: 135 - protein, 21 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(20): Xaa can be any naturally occurring amino acid
XXXCCXXXXXXXXXXXXXXXC

SEQ ID NO: 136 - protein, 22 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(21): Xaa can be any naturally occurring amino acid
XXXCCXXXXXXXXXXXXXXXXC

SEQ ID NO: 137 - protein, 13 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(12): Xaa can be any naturally occurring amino acid
XXXCCXXXXXXXC

SEQ ID NO: 138 - protein, 14 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(13): Xaa can be any naturally occurring amino acid
XXXCCXXXXXXXXC

SEQ ID NO: 139 - protein, 15 aa, Artificial Sequence
  N-terminal motif
  MISC_FEATURE (1)..(1): X is Leu, Ile, Val, or Phe
  misc_feature (2)..(3): Xaa can be any naturally occurring amino acid
  misc_feature (6)..(14): Xaa can be any naturally occurring amino acid
XXXCCXXXXXXXXXC

SEQ ID NO: 140 - protein, 117 aa, Artificial Sequence
  Ena1B-DE-HA insertion variant
MGNCSTNLSCCANGQKTIVQDKVCIDWTAAATAAIIYADNISQDIYASGYLKVDTGSGPVTIVFYSGGVTGTAVETIVVATGSSASFTVRRFDTVTILGTAAAETGEFCMTIRYTLS

SEQ ID NO: 141 - protein, 127 aa, Artificial Sequence
  Ena1B-DE-Flag insertion variant
MGNCSTNLSCCANGQKTIVQDKVCIDWTAAATAAIIYADNISQDIYASGYLKVDTGSDYKDDDDKGSGPVTIVFYSGGVTGTAVETIVVATGSSASFTVRRFDTVTILGTAAAETGEFCMTIRYTLS

SEQ ID NO: 142 - protein, 128 aa, Artificial Sequence
  Ena1B-HI-HA insertion variant
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGSYPYDVPDYAGSAAETGEFCMTIRYTLS

SEQ ID NO: 143 - protein, 9 aa, Artificial Sequence
  HA-tag
YPYDVPDYA

SEQ ID NO: 144 - protein, 8 aa, Artificial Sequence
  FLAG-tag
DYKDDDDK

SEQ ID NO: 145 - protein, 122 aa, Bacillus thuringiensis
MSCECSGSALTCCPDKNYVQDKVCSPWSATVVATAIDNVLYTNNINQNVVGTGFVKYDVGPGPITVEALDSAGGTIDTQTLNPGTSIAFTYRRFDSIQVVLPATPAGTYQGEFCITTRYPLS

SEQ ID NO: 146 - protein, 141 aa, Bacillus thuringiensis
MKNKKNIGCFAPLSIICPDPCPPPPPPNPNCERVTNEFAGNFLITNNTIPSAKDASQSMILWQSDGILLISGTVSVYNSTSSTETITIQIVGTVTNIFTVFPGNTISYTGKDLHSVSIINITSNPSLYLEGKYCCEFTCCL

SEQ ID NO: 147 - protein, 6 aa, Artificial Sequence
  Conserved motif
  misc_feature (5)..(5): Xaa can be any naturally occurring amino acid
SLNYXF

SEQ ID NO: 148 - protein, 6 aa, Artificial Sequence
  Conserved motif
  misc_feature (5)..(5): Xaa can be any naturally occurring amino acid
SLNYXY

SEQ ID NO: 149 - protein, 6 aa, Artificial Sequence
  Conserved motif
  misc_feature (5)..(5): Xaa can be any naturally occurring amino acid
SINYXF

SEQ ID NO: 150 - protein, 6 aa, Artificial Sequence
  Conserved motif
  misc_feature (5)..(5): Xaa can be any naturally occurring amino acid
SINYXY

(19) United States
(12) Patent Application Publication
(10) Pub. No.: US 2023/0279059 A1
(43) Pub. Date: Sep. 7, 2023

(54) NOVEL BACTERIAL PROTEIN FIBERS

(71) Applicants: VIB VZW, Gent (BE); VRIJE UNIVERSITEIT BRUSSEL, Brussel (BE); NORWEGIAN UNIVERSITY OF LIFE SCIENCES, Oslo (NO)

(72) Inventors: Han Remaut, Roosbeek (BE); Mike Sleutel, Huizingen (BE); Marina Aspholm, Oslo (NO); Brajabandhu Pradhan, Brussel (BE)

(21) Appl. No.: 18/020,068

(22) PCT Filed: Aug. 6, 2021
(86) PCT No.: PCT/EP2021/072085

§ 371 Date: Feb. 6, 2023

(30) Foreign Application Priority Data
Aug. 7, 2020 (EP) 20189961.4

(51) Int. Cl. C07K 14/32; C12N 15/63

(52) U.S. Cl. CPC C07K 14/32; C12N 15/63; C12N 2310/35

(57) ABSTRACT

The present invention relates to the field of Bacillus endospore appendages (Ena) and new protein multimeric and fibrous assemblies for applications as bionanomaterials. In particular, the invention relates to self-assembling proteins composed of bacterial DUF3992 domain-containing protein subunits, containing a conserved N-terminal cysteine-containing region, and engineered proteins, as well as multimers and fibers thereof. Moreover, recombinant expression of said self-assembling protein subunits provides for production methods of novel protein nanofibers and modified display surfaces, such as Bacillus spores. Finally, the use of said multimers, fibers, and surfaces in biomedical and biotechnological applications is described herein.

### CROSS-REFERENCE TO RELATED APPLICATIONS

This application is a national phase entry under 35 U.S.C. § 371 of International Patent Application PCT/EP2021/072085, filed Aug. 6, 2021, designating the United States of America and published in English as International Patent Publication WO 2022/029325 on Feb. 10, 2022, which claims the benefit under Article 8 of the Patent Cooperation Treaty to European Patent Application Serial No. 20189961.4, filed Aug. 7, 2020, the entireties of which are hereby incorporated by reference.

### FIELD OF THE INVENTION

The present invention relates to the field of Bacillus endospore appendages (Ena) and new protein multimeric and fibrous assemblies for applications as bionanomaterials. In particular, the invention relates to self-assembling proteins composed of bacterial DUF3992 domain-containing protein subunits, containing a conserved N-terminal cysteine-containing region, and engineered proteins, as well as multimers and fibers thereof. Moreover, recombinant expression of said self-assembling protein subunits provides for production methods of novel protein nanofibers and modified display surfaces, such as Bacillus spores. Finally, the use of said multimers, fibers, and surfaces in biomedical and biotechnological applications is described herein.

### BACKGROUND

Self-assembling molecules provide the challenging opportunity to control chemical functionality and morphology and thus biological activity. The unique properties of proteins including their modular nature, biocompatibility, and biodegradability offer exciting opportunities in designing smart nanomaterials (Herrera Estrada & Champion, 2015; Jain et al., 2018). Inspired by nature, several proteins/peptides have been engineered to self-assemble into a variety of complex structures, ranging from nanoparticles, vesicles, cages and fibrous assemblies; these can be endowed with novel functionalities offering numerous applications in diverse areas of bioengineering (Matsuurua 2014; Katyal et al., 2019). Varying the amino acid sequences of self-assembling peptides and proteins and manipulating the environmental parameters, allows to modulate the properties, and to control self-assembly to obtain diverse on demand supramolecular nanostructures (Lombardi et al., 2019). The various properties of the side chains in amino acids offer possibilities for their chemical modification with infinite sequence combinations, as well as modifying the amine- and/or carboxy-termini of proteins can tune the self-assembly of protein polymers into specific nanoarchitectures (Aluri et al., 2012; Yu et al., 1996). So natural self-assembling proteins or peptides may be engineered to induce various properties other than self-assembly, including self-healing, shear-thinning, shape memory, and so on (Chen and Zou, 2019).

When faced with adverse growth conditions, bacteria belonging to the phylum Firmicutes can differentiate into the metabolically dormant and non-productive endospore state. These endospores exhibit extreme resilience towards environmental stressors due to their dehydrated state and unique multilayered cellular structure, and can germinate into the metabolically active and replicating vegetative growth state even hundreds of years after their formation (Setlow, 2014). In this way, Firmicutes belonging to the classes Bacilli and Clostridia are able to withstand long periods of drought, starvation, high oxygen or antibiotic stress. Endospores typically consist of an innermost dehydrated core which contains the bacterial DNA. The core is enclosed by an inner membrane surrounded by a thin layer of peptidoglycan that will function as the cell wall of the vegetative cell that emerges during spore germination. Then comes a thick cortex layer of modified peptidoglycan that is essential for dormancy (Atrih and Foster, 1999). The cortex layer is in turn surrounded by several proteinaceous coat layers. In some Clostridium and most Bacillus cereus group species, the spore is enclosed by an outermost loose-fitting paracrystalline exosporium layer consisting of (glyco)proteins and lipids (Stewart, 2015). The surface of Bacillus and Clostridium endospores can also be decorated with multiple micrometers long and a few nanometers wide filamentous appendages, which show a great structural diversity between strains and species (Hachisuka and Kuno, 1976; Rode et al., 1971; Walker et al., 2007). Bacillus cereus sensu lato is a group of Gram-positive endospore-forming bacteria that displays a high ecological diversity notwithstanding their phylogenetic relationship. Their endospores exhibit extreme resilience towards environmental stressors due to their dehydrated state and unique multilayered cellular structure and can germinate into the metabolically active and replicating vegetative growth state even hundreds of years after their formation (Setlow, 2014). B. cereus endospores are decorated with micrometer-long appendages of unknown identity and function. The number of endospore appendages (hereafter called Enas) varies and morphology between B. cereus group strains and species and some strains even simultaneously express Enas of different morphologies (Smirnova et al., 2013). Structures resembling the Enas have not been observed on the surface of the vegetative cells suggesting that they represent spore-specific fibers. Enas appear to be a widespread feature among spores of strains belonging to the B. cereus group. Ankolekar et al., showed that all of 47 food isolates of B. cereus produced endospores with appendages (Ankolekar & Labbe, 2010). Appendages were also found on spores of ten out of twelve food-borne, enterotoxigenic isolates of Bacillus thuringiensis, which is closely related to B. cereus, and best known for its insecticidal activity (Ankolekar & Labbe, 2010). Altogether, this makes those Ena structures an interesting starting point for engineering towards new sustainable biomaterials. Remarkably, the presence of spore appendages in species belonging to the B. cereus group was reported already in the '60s but efforts to characterize their composition and genetic identity has failed due to difficulties to solubilize and enzymatically digest the fibers (Gerhardt & Ribi, 1964; DesRosier & Lara, 1981). So, there is an interest and need for the structural characterization of such endospore appendages to allow the design, development, and production of novel types of smart biomaterials with improved properties such as sustainability in harsh environmental conditions.

### SUMMARY OF THE INVENTION

The present invention is based on the resolution of the genetic and structural basis of isolated endospore appendages (Enas) from the food poisoning outbreak strain B. cereus NVH-0075/95, which revealed proteinaceous fibers of two main morphologies, S-type and L-type fibers. By using cryo-EM and 3D helical reconstruction it was shown that Bacillus endospore appendages (Enas) form a novel class of Gram-positive pili, characterized by subunits with a jellyroll topology forming multimers that are laterally stacked by β-sheet augmentation. Moreover, Ena fibers are longitudinally stabilized by disulphide crosslinking through extension of their N-terminal protein subunit peptides that bridge the multimers resulting in flexible pili (see also FIG. 2) that are highly resistant to heat, drought and chemical damage. The 3D structure allowed to deduce that Ena fibers are composed of a protein family of bacterial DUF3992 domain-containing proteins with a so far unknown function, and a conserved N-terminal region for each family member, which were herein annotated for the first time as ‘Ena’ proteins. The genetic identity of S-type and L-type fiber constituents was confirmed by analysis of mutants lacking genes encoding potential Ena protein subunits. Phylogenetic analyses show that the S-type ena fibers are encoded by a di-cistronic operon that is uniquely present in a subset of species belonging to the B. cereus group and revealed the presence of defined ena clades amongst different eco- and pathotypes, with these Ena genes having the commonality to encode Ena proteins, characterized by an N-terminal region with at least two conserved Cysteine residues and a spacer region (see FIG. 8), followed by a DUF3992 domain, to allow self-assembly into folded structures as defined herein, resulting in multimeric or fibrous assemblies. In vivo, the subunits encoded in the Ena operons are interdependent for the assembly of Enas. Surprisingly, recombinantly expressed Ena proteins can be made to individually self-assemble into protein nanofibers with properties and structure similar to those of in vivo Enas. Enas thus represent a novel class of pili specifically adapted to the harsh conditions encountered by bacterial spores, and by revealing the genetic and structural basis, the insights on how to produce modified spores, or modified and engineered Ena protomers or multimers to provide for protein assemblies such as discs or helices applicable as next-generation biomaterials, are established herein.

The first aspect of the invention relates to a protein with self-assembling properties, which is characterized in its amino acid sequence as belonging to the PFAM13157 class, i.e. characterized by the presence of a DUF3992 domain in its sequence, and which further requires to match the 3D structural fold of an Ena protein, as presented herein, specifically the fold of Ena1B (with a sequence depicted in SEQ ID NO:8), with a highly significant similarity score, defined as a Dali Z-score of 6 or more, 6.5 or more, or preferably n/10-4 or more, wherein n is the number of amino acids of said protein sequence. In one embodiment, said self-assembling protein subunit is provided by the bacterially originating proteins comprising an amino acid sequence selected from the group of SEQ ID NOs:1-80, SEQ ID NO:145 and SEQ ID NO:146, representing the Ena protein sequences identified in the present application, or any prokaryotic homologue with at least 60%, or at least 70% or at least 80% or at least 90% identity of any one of the sequences of SEQ ID NO:1-80, SEQ ID NO:145 or SEQ ID NO:146, wherein the % identity is calculated over the full length window of the sequence. In fact, the structural requirement described herein to match the Ena1B fold as disclosed herein often still stands for bacterial proteins with homologies even lower than 60% identity to the structural reference sequence of SEQ ID NO:8, since the bacterial Ena family is further classified in different members, as described below. So one embodiment relates to the isolated self-assembling protein comprising a DUF3992 domain, as determined by aligning to its Hidden Markov Model as depicted in Table 1, and wherein said protein subunit has a 3D (predicted) fold matching the Ena1B structure with a fold similarity score of 6.5 or more, as defined herein, and wherein Ena1B corresponds to SEQ ID NO:8 and wherein the Ena1B reference structure corresponds to the coordinates as provided herein in Table 2, and as deposited in PDB7A02.

In a specific embodiment, the self-assembling proteins referred to herein relates to said Ena protein family, as defined above, and/or as provided by the amino acid sequences depicted in SEQ ID NOs: 1-80, SEQ ID NO:145, or SEQ ID NO:146, providing representative examples of the Bacillus Ena1A (SEQ ID NO: 1-7), Ena1B (SEQ ID NO: 8-14), Ena1C (SEQ ID NO: 15-20), Bacillus Ena2A (SEQ ID NO: 21-28, SEQ ID NO:145), Ena2B (SEQ ID NO: 29-37), Ena2C (SEQ ID NO: 38-48, SEQ ID NO:146), and different types of other Bacillus Ena3 (SEQ ID NO: 49-80) proteins, respectively, or bacterial orthologues of any one thereof, which have at least 80% identity of any sequence depicted in SEQ ID NO:1-80, SEQ ID NO:145 or SEQ ID NO:146. The regions and level of sequence conservation is shown for the Ena family members by the multiple sequence alignments depicted in FIGS. 16-19.

A further embodiment relates to said self-assembling protein as described herein, which is an engineered self-assembling protein, wherein the Ena fold and HMM profile as described herein matches the Ena1B fold and DUF3992 profile, as described herein, but which is ‘engineered’ or ‘modified’ by further comprising for example, but not limited to, at least one of the modifications including a heterologous N- or C-terminal tag, and/or a steric block, a protein sequence variant which may contain one or more mutations as compared to the native or wild type Ena sequence, or which may contain an insertion of a peptide or scaffold, or a deletion of a number of amino acids, or which may be provided as separate parts of the Ena protein, such as ‘split’ parts, that assemble upon co-incubation.

A second aspect of the invention relates to a protein multimer comprising or containing at least seven of said self-assembling protein subunits, and preferably between 7 and maximally twelve subunits, which are non-covalently linked. More specifically, said multimer consists of seven, eight, nine, ten, eleven, twelve, 13, 14, 15, 16, 17, 18, 19, 20, or more self-assembling Ena protein subunits as defined herein, non-covalently stacked via β-sheet augmentation (a protein-protein interaction principle described in Remaut and Waksman, 2006). In a specific embodiment, said multimers as described herein may further comprise covalent connections, provided by for instance Cys connections between different protein subunits of said multimer (in suitable conditions). In one embodiment, said multimers are present ‘as such’, i.e. not as a filament or fiber constellation, and are therefore non-naturally occurring multimeric assemblies. Particularly, said self-assembling protein subunits defined herein as Ena proteins, may further comprise at least two conserved cysteine residues in their N-terminal region or N-terminal connector, as used interchangeably herein, for intermolecular disulphide bridge formation with further multimers. In a specific embodiment the multimeric assembly comprises seven to twelve protein subunits from the Ena protein family, as further defined herein, or as provided by the amino acid sequences depicted in SEQ ID NOs: 1-80, SEQ ID NO:145, or SEQ ID NO:146 providing representative examples of the Bacillus Ena1A (SEQ ID NO: 1-7), Ena1B (SEQ ID NO: 8-14), Ena1C (SEQ ID NO: 15-20), Bacillus Ena2A (SEQ ID NO: 21-28, SEQ ID NO:145), Ena2B (SEQ ID NO: 29-37), Ena2C (SEQ ID NO: 38-48, SEQ ID NO:146), and different types of other Bacillus Ena3 (SEQ ID NO: 49-80) proteins respectively, or bacterial orthologues thereof, which have at least 80% identity of any sequence depicted in SEQ ID NO:1-80, SEQ ID NO:145, or SEQ ID NO:146. A specific embodiment relates to said multimers with 7 to 12 protein subunits with identical self-assembling proteins as described herein. Alternatively, the multimers comprise at least 7 protein subunits wherein at least one of said protein subunits is an engineered self-assembling Ena protein, as defined herein and which concerns a non-naturally occurring Ena protein. In a specific embodiment, said multimers comprise at least 7, preferably maximally 12 Ena protein subunits, wherein at least one subunit is an engineered Ena protein comprising a steric block at the N- and/or C-terminus, thereby preventing the multimer to further assemble into fibers (FIG. 14). In a specific embodiment said N- or C-terminal steric block is a heterologous N- and/or C-terminal tag. In a specific embodiment said heterologous N- and/or C-terminal tag or extension to form such as steric block is minimally 1, 2, 3, 4, 5, preferably 6, or more amino acid residues. Certain embodiments relate to said multimers wherein said Ena protein subunits may be identical or different self-assembling Ena proteins wherein at least one of them is engineered to comprise a heterologous N- and/or C-terminal tag. Alternatively, said at least one engineered Ena protein subunit may be an Ena mutant protein variant, or may be an Ena protein that is a fusion protein, or containing an inserted peptide or protein domain at exposed loops, as exemplified and described in FIG. 15 and outlined in the Example section.

A specific embodiment relates to said multimers as described herein which are homomultimers or heteromultimers, and more specifically relate to multimers consisting of 6, or 7 to 12 subunits, and preferably relate to a heptamer, so consisting of 7 subunits, or a nonamer, so consisting of 9 subunits, both thereby possibly forming a disc-like multimer, or a decamer, undecamer or dodecamer, so consisting of 10, 11 or 12 subunits, respectively, thereby forming a helical turn or an arc of a β-propeller structure (FIG. 14).

Another embodiment relates to said self-assembling protein subunits, or multimers of self-assembling DUF3992-containing protein subunits or Ena protein subunits or engineered Ena protein subunits, which comprise an N-terminal region or N-terminal connector (Ntc) region wherein the amino acid residue consensus motif ZXₙCCXₘC is present, wherein X is any amino acid, n is 1 or 2, m is between 10-12, and Z is preferably Leu, Ile, Val or Phe, and preferably wherein the C-terminal region or C-terminal receiver region comprises the consensus motif GX_{2/3}CX₄Y, wherein G is Glycine, X is any amino acid (2 or 3 residues), and Y is Tyrosine, so that the Cysteines (C) present in said N- and C-terminal region motifs of the protein subunits may form disulphide bridges for longitudinally connecting one multimer to another multimer (ultimately leading to assemblies into S-fibers as in FIG. 14A; FIG. 16-17). A further alternative embodiment relates to engineered self-assembling protein subunits or multimers comprising an N-terminal connector region with the motif ZXₙCCXₘC as defined herein, but with a shorter N-terminal spacer region wherein the m is 7 to 9, or a longer N-terminal spacer region wherein m is 13-16. Said engineered multimers will upon self-assembly result in fibers with lower flexibility or increased rigidity as compared to assembled fibers with multimers wherein m is 10 to 12 for said spacer region. A further alternative embodiment relates to said self-assembling protein subunits or multimers constituted by said Ena protein subunits, which comprise an N-terminal region or N-terminal connector region wherein the amino acid residue consensus motif ZXₙC(C)XₘC is present, wherein X is any amino acid, n is 1 or 2, m is between 10-12, and Z is preferably Leu, Ile, Val or Phe, C is cys and (C) is an optional Cys, meaning that one or 2 cys are present in said motif for these Ena proteins (ultimately classified further herein as Ena3 proteins), and preferably wherein the C-terminal region or C-terminal receiver region comprises the consensus motif S-Z-N-Y-X-B, wherein Z is Leu or Ile, B is Phe or Tyr, and X is any amino acid, so that the Cysteines (C) present in said N- and C-terminal region motifs of the protein subunits may form disulphide bridges for longitudinally connecting one multimer to another multimer (ultimately leading to assemblies into L-fibers as in FIG. 14B; FIG. 19).

Another aspect of the invention relates to protein fibers produced as to comprise at least two of said multimers as described herein, wherein said multimers are not hindered to longitudinally crosslink through disulphide bonds, more specifically through at least one disulphide bond, preferably two or more disulphide bonds. Said disulphide bonds may be formed between side chains of cysteine residues of the N-terminal region or N-terminal connector of one or more subunits of a multimer with one or more cysteine residues present in the N- and/or C-terminal region of one or more subunits of the multimer constituting the preceding layer of the longitudinally formed protein fiber. Said protein fiber may be a recombinantly produced fiber.

In another embodiment, said protein fiber is an engineered protein fiber, comprising at least two multimers of which at least one multimer is an engineered multimer as defined herein, or wherein at least one multimer comprises at least one engineered Ena protein, as defined herein. In a preferred embodiment the protein fibers comprises multimers wherein the protein subunits comprise identical self-assembling protein subunits as described herein, and/or are composed of identical Ena proteins.

Another aspect of the invention relates to a chimeric gene construct comprising a promoter or regulatory sequence element that is operably linked to a DNA element comprising a coding sequence for the (engineered) self-assembling protein, preferably an Ena protein, as defined herein. More specifically, said coding sequence may code for a protein comprising an Ena protein as depicted in SEQ ID NOs: 1-80; SEQ ID NO:145, or SEQ ID NO:146, or a functional homologue of any of said Ena family members comprising Ena1/2A, Ena1/2B, Ena1/2C, or Ena3A, with at least 80% amino acid identity to any of SEQ ID NO:1-80, SEQ ID NO:145, or SEQ ID NO:146, or may code for an engineered Ena protein form thereof, as defined herein. In a specific embodiment, said promoter or regulatory element is heterologous to the coding sequence where it is operably linked to, and optionally is an inducible promoter, as known in the art.

A further embodiment relates to a host cell for expression of the chimeric gene as described herein, or for expression of the self-assembling protomers of the multimers or protein assemblies as described herein. Another embodiment relates to a modified spore-forming cell or bacterium, comprising the chimeric gene as described herein, or an engineered Ena gene or a gene encoding an engineered Ena protein. Another embodiment relates to a modified bacterial spore, in particular a modified Bacillus endospore, which comprises and/or displays Ena proteins, or engineered forms thereof, or multimers as described herein, or has protein fibers, in particular engineered or modified protein fibers, recombinantly produced fibers or spores, as described herein.

In a further aspect of the invention a modified surface or solid support is provided, said surface comprising an Ena protein, a multimer assembly, or a protein fiber as described herein, or an engineered form of any thereof. Said modified surface is composed by covalent attachments of said Ena protein, multimer or fiber to said surface, and may be a cellular or artificial surface, in particular a solid surface of any material type. Said modified surface may thus be used as a nucleator for epitaxial growth of a protein fiber, for instance when said modified surface is exposed or contacted with a solution of Ena proteins, wherein said Ena proteins are preferably present in monomeric or oligomeric form.

Further embodiments relate to a protein film comprising the engineered Ena protein fiber and/or the Ena protein fibers as described herein, said film preferably being a thin film, as known in the art. Alternatively a hydrogel is disclosed herein comprising the engineered protein fiber as described herein and/or the Ena protein fiber as described herein. A further embodiment relates to a nanowire comprising the engineered protein fibers that are spun into a thicker, thread-like bundle.

A final aspect of the invention relates to method to recombinantly produce the protein assemblies as described herein, more particularly the Ena proteins, multimeric and fibrous assemblies, or modified surfaces, in particular spore surfaces or synthetic surfaces as described herein.

One embodiment describes a method to produce a self-assembling DUF3992 domain-containing monomer, or multimer as described herein comprising the steps of:
a) expressing a chimeric gene construct as described herein in a host cell, or using the host cell as described herein, wherein the self-assembling protein subunit optionally comprises an N- and/or C-terminal tag, and (optionally)
b) purifying the self-assembled DUF3992-domain-containing proteins or multimers, the latter being formed after oligomerisation of the expressed protein subunits.

Another embodiment provides for a method to recombinantly produce the self-assembling DUF3992 domain-containing or Ena proteins which are arrested or at least impeded in fiber assembly or in epitaxial growth, so a method to recombinantly produce engineered Ena proteins blocked in fiber outgrowth, comprising the method as described above, wherein the N- and/or C-terminal tag is at least 1, preferably at least 6, more preferably at least 9, or 15 amino acids in length to sterically block self-assembly of the protein subunits or multimers in longitudinal fiber formation. In a further embodiment, said N- or C-terminal tag is at least 6 amino acids in length to reversibly impede or hamper self-assembly of the protein subunits or multimers in longitudinal rigid fiber formation. In said case the N- or C-terminal tag may be a removable tag, for instance, by including a protease recognition sequence for removal of the tag by a protease, and reversal of the steric blockage of subunit and multimer assembly.

Another embodiment relates to a method to produce a protein fiber as described herein, comprising the steps a) and b) of the above method, wherein the N- and/or C-terminal tag is a present as a removable or cleavable tag, said method further comprising the step c) wherein the N- and/or C-terminal tag is removed or cleaved off to allow further self-assembly of the formed multimers into protein fibers. Alternatively step c) may be exerted prior to the purification step b). Furthermore, a method is provided to produce the modified surface as described herein, comprising the steps a), b), and/or c) (or vice versa c) and/or b)), further comprising step d) wherein a surface is modified by displaying or covalently attaching the (engineered) Ena protein, multimer or fiber to said surface.

Finally, the protein assemblies, such as fibers as described herein, may be produced within a cell, as depicted in the method for recombinant production of the Ena protein fibers comprising the steps of:
a) expressing the chimeric gene construct as described herein in a host cell, or using the host cell as described herein, or expressing an Ena protein, or an engineered Ena protein, as described herein, wherein the protein subunit does not have a steric block, so the self-assembling protein consisting of a wild-type or engineered self-assembling Ena protein with a free N-terminal connector, and (optionally)
b) isolation of the Ena protein assemblies, such as fiber or multimers, formed after oligomerisation of the expressed protein subunits within the cytoplasm.

### DESCRIPTION OF THE FIGURES

The drawings described are only schematic and are non-limiting. In the drawings, the size of some of the elements may be exaggerated and not drawn on scale for illustrative purposes.

The drawings described are only schematic and are non-limiting. In the drawings, the size of some of the elements may be exaggerated and not drawn on scale for illustrative purposes.

FIGS. 1A-1E. Bacillus cereus endospores carry S and 1-type Enas.

(FIGS. 1A and 1B) negative stain TEM image of B. cereus NVH 0075/95 endospore, showing spore body (SB), exosporium (E), and endospore appendages (Ena), which emerge from the endospore individually or as fiber clusters (boxed). At the distal end, Enas terminate in a single or multiple thin ruffles (R). (FIGS. 1C AND 1D) Single fiber cryoTEM images and negative stain 2D class averages of S-type (FIG. 1C) and L-type Enas (FIG. 1D). (FIG. 1E) Length distribution of S- and L-type Enas and number of Enas per endospore (inset), (n=1023, from 150 endospores, from 5 batches). See also FIG. 7.

FIGS. 2A-2E. CryoTEM structure of S-type Enas.

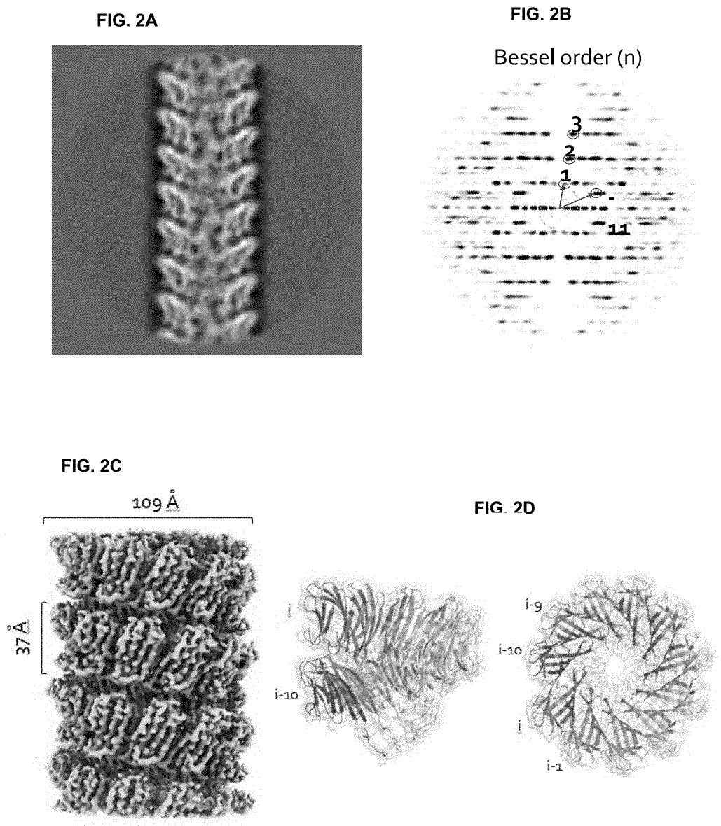

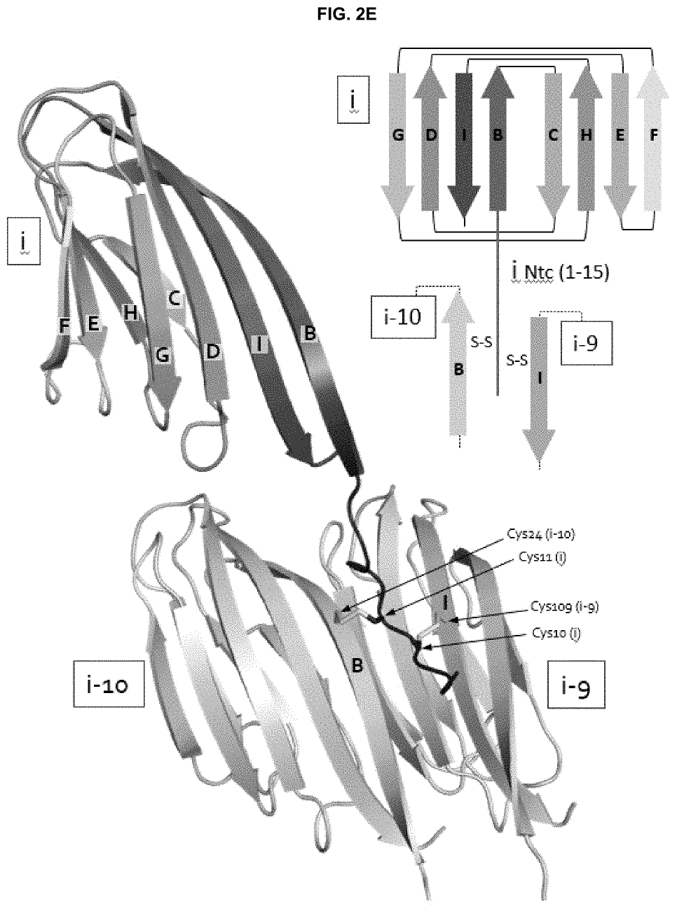

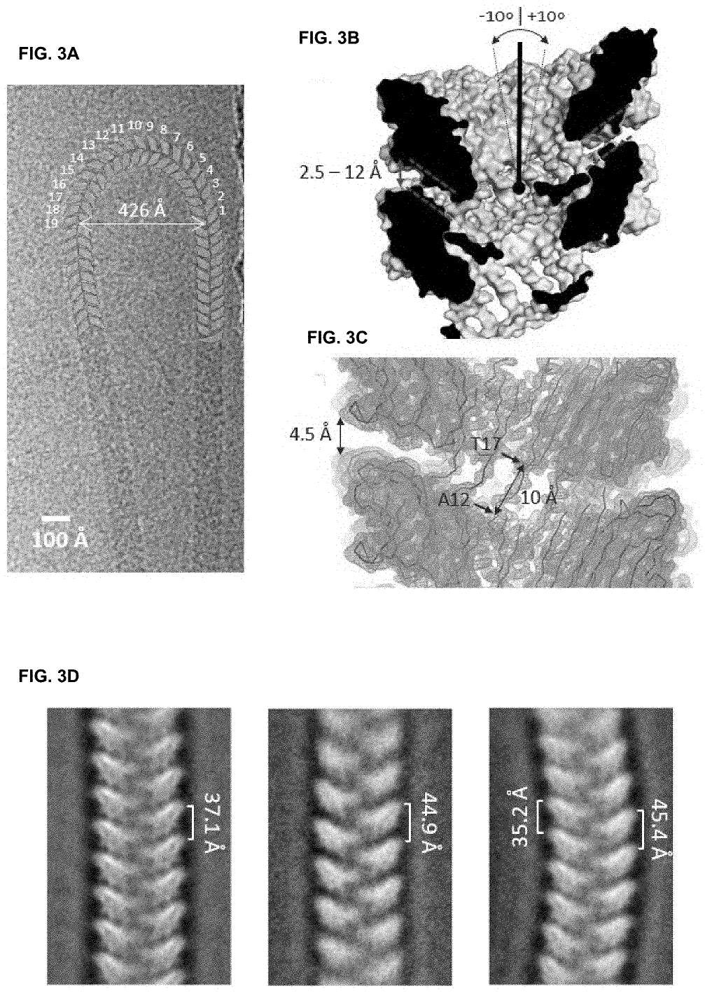

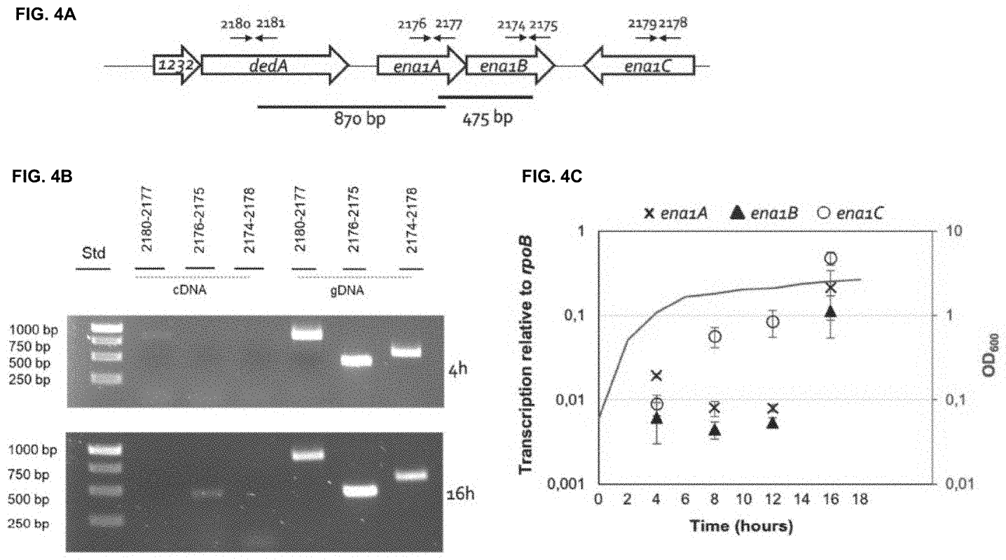

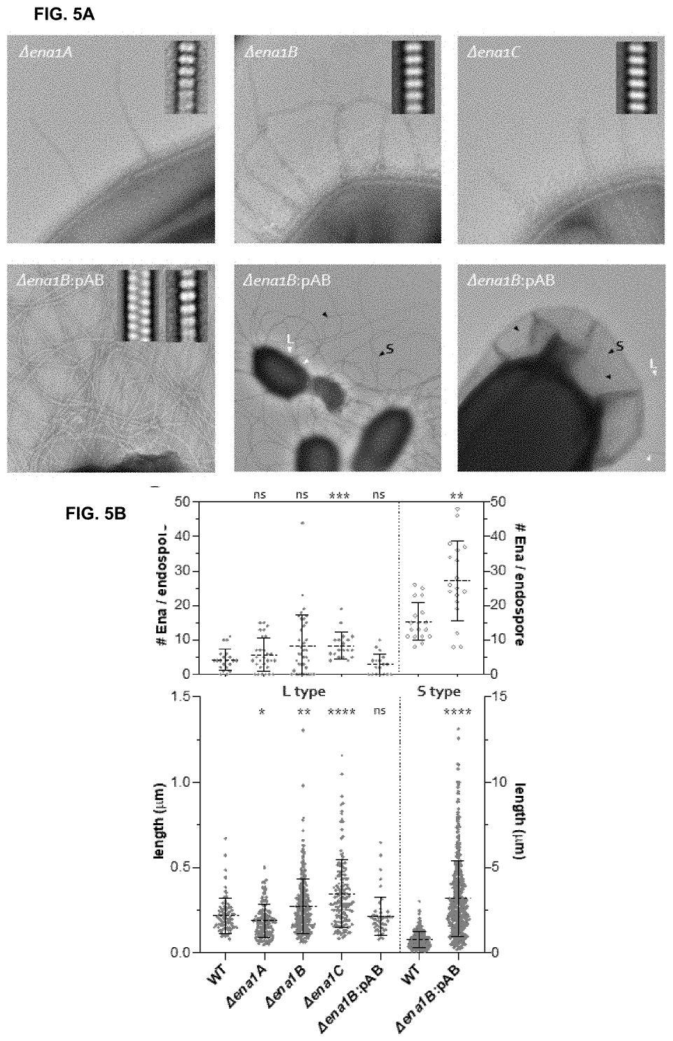

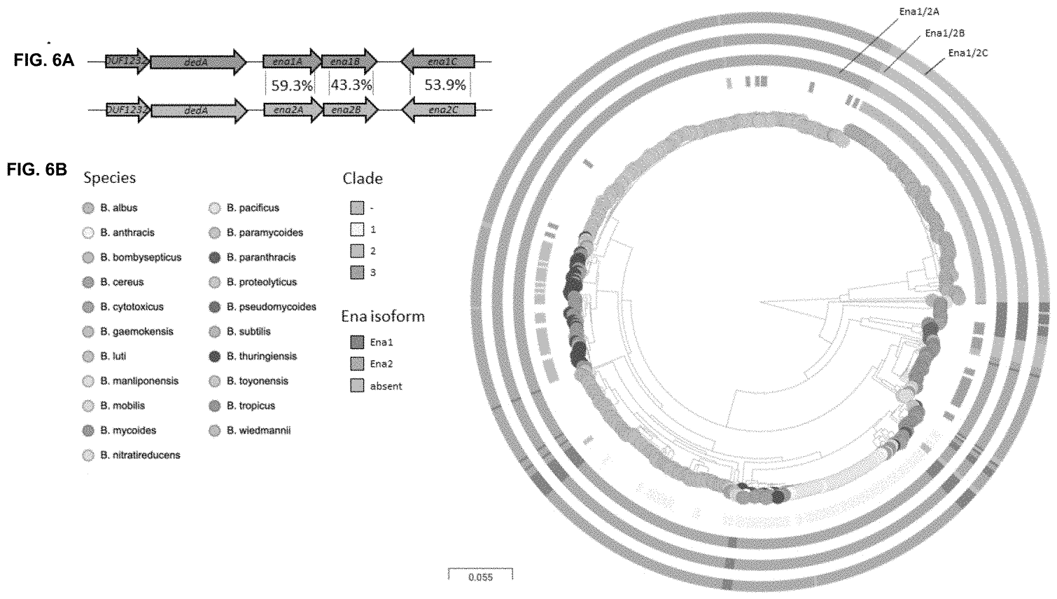

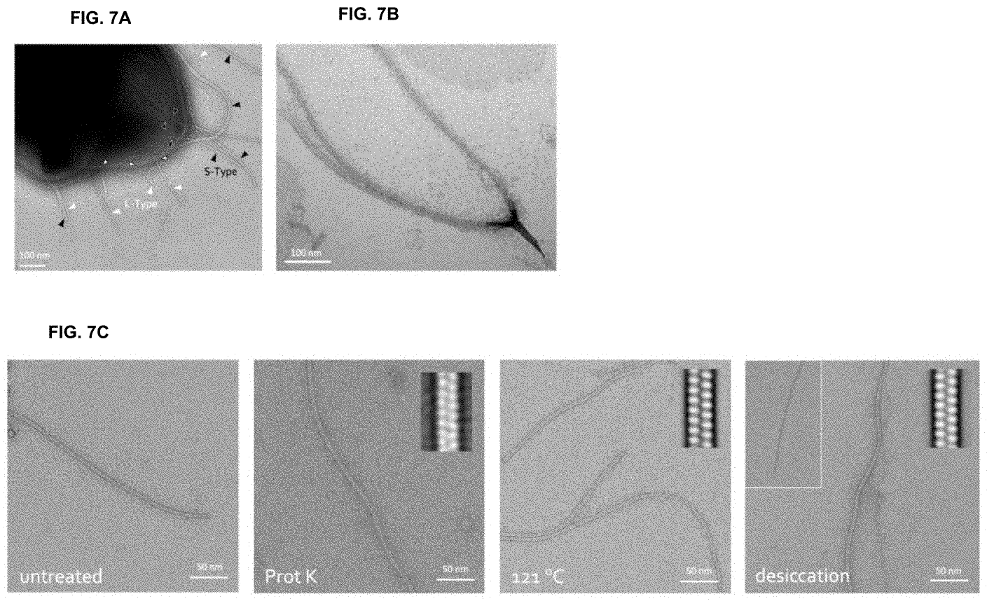

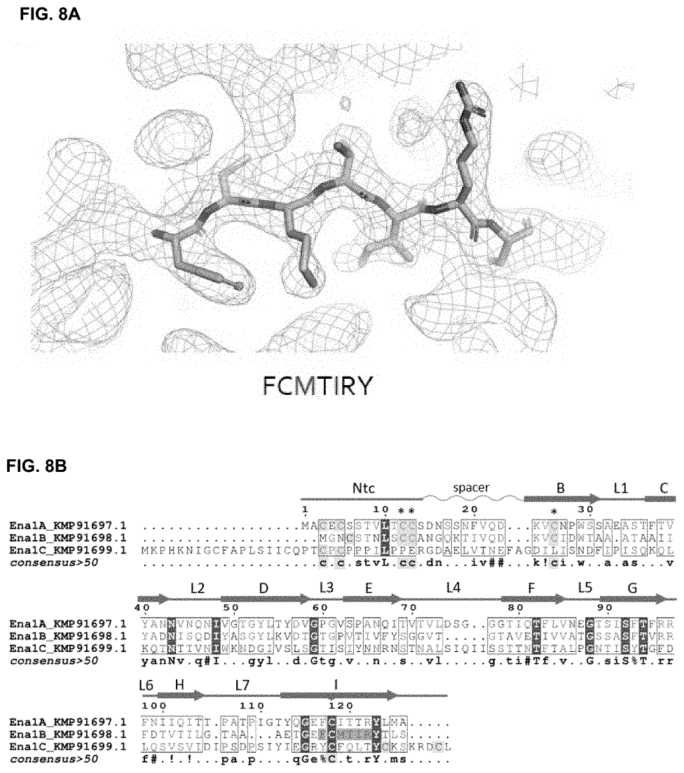

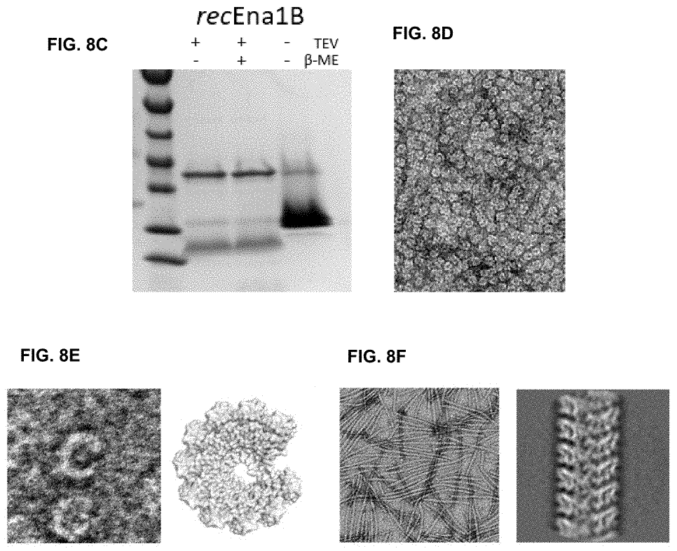

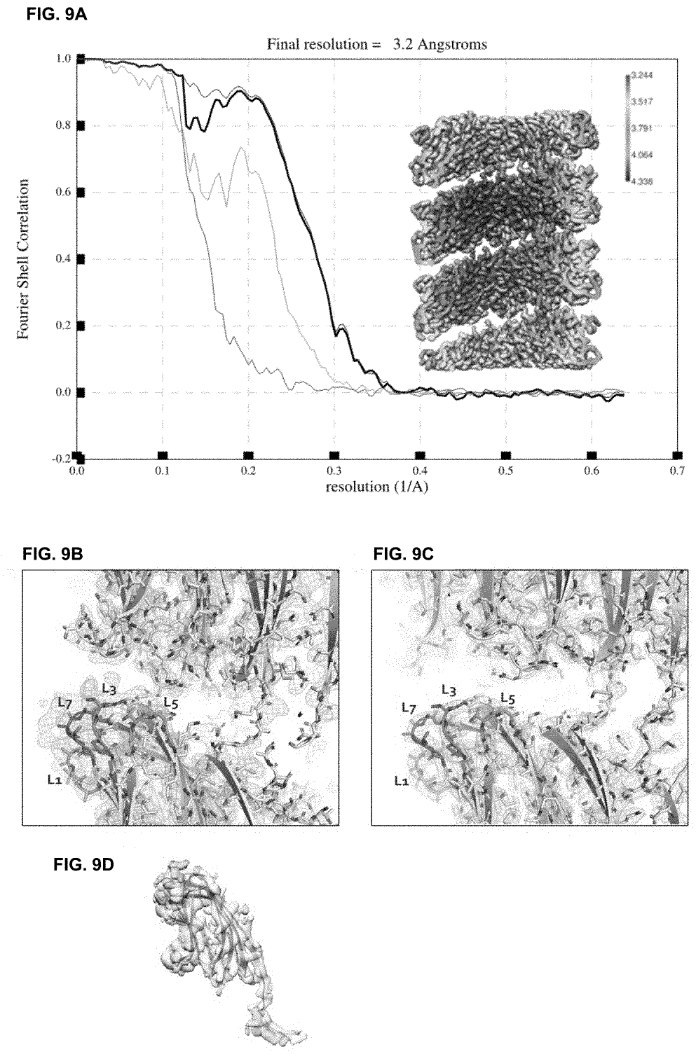

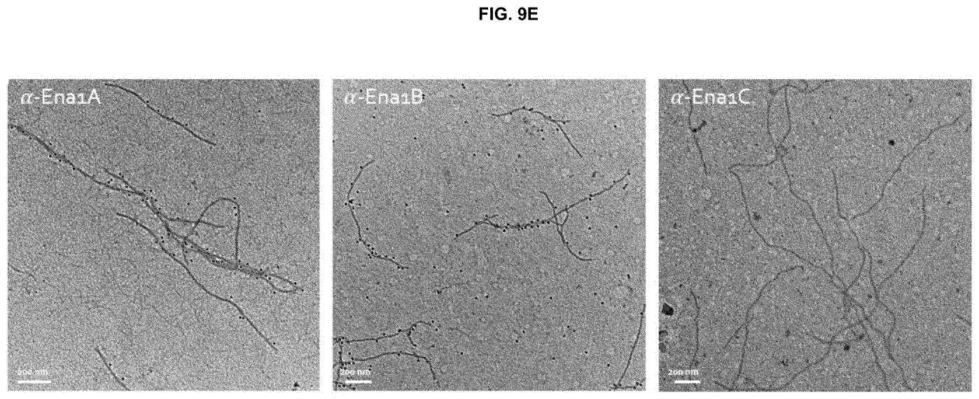

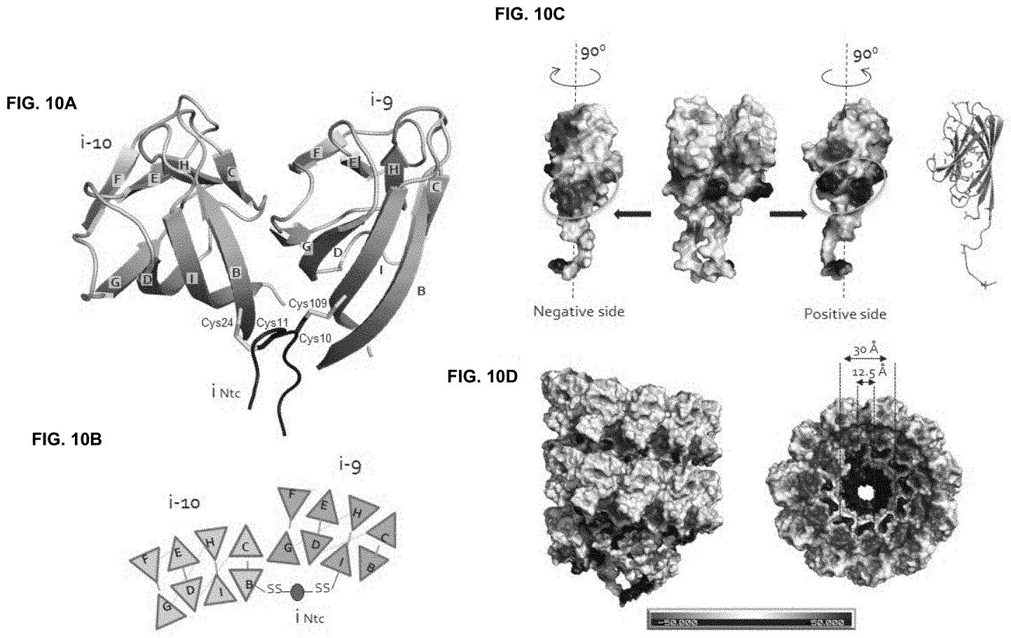

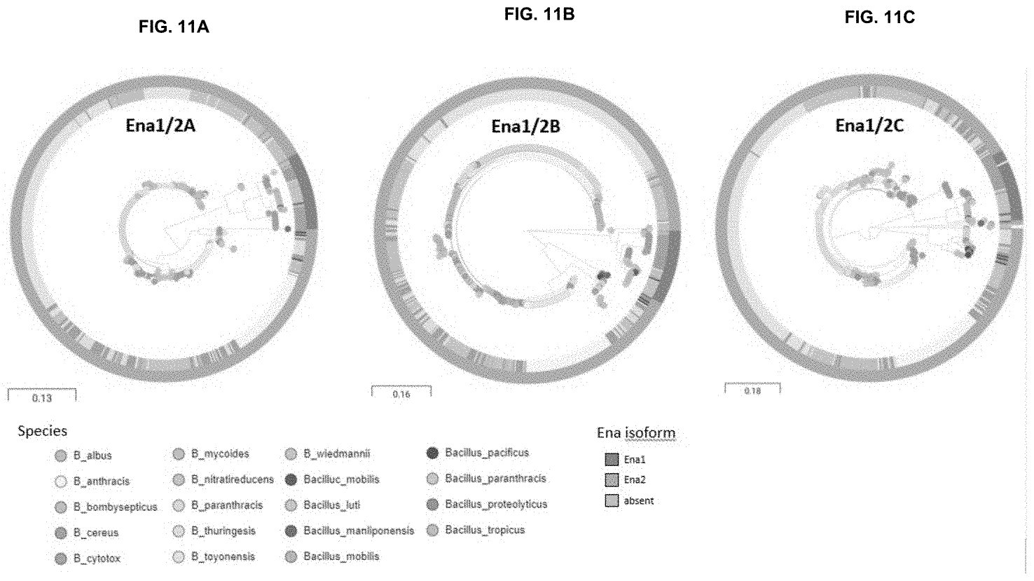

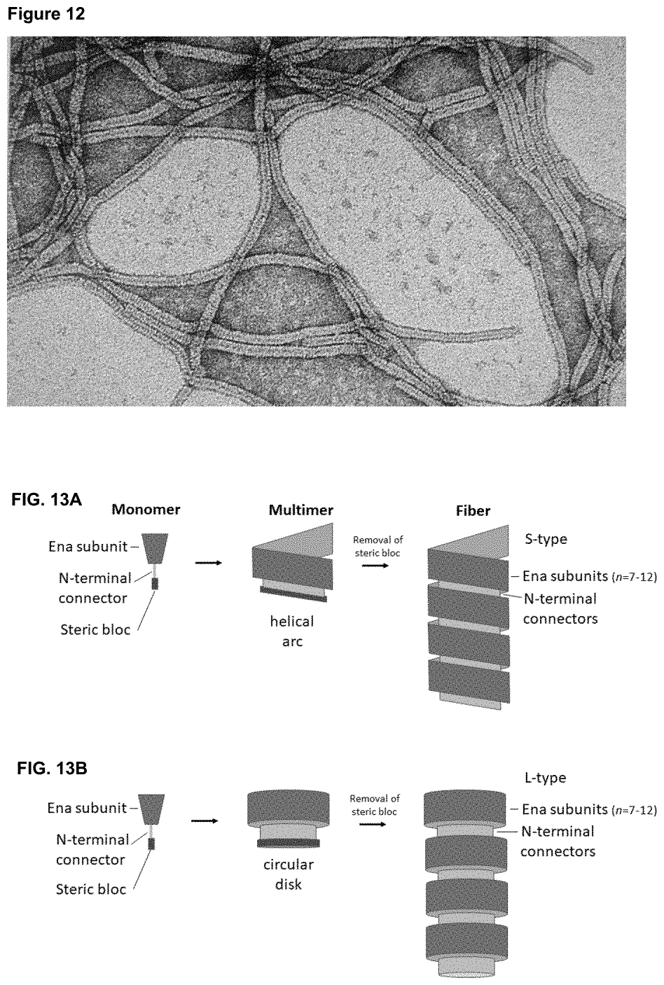

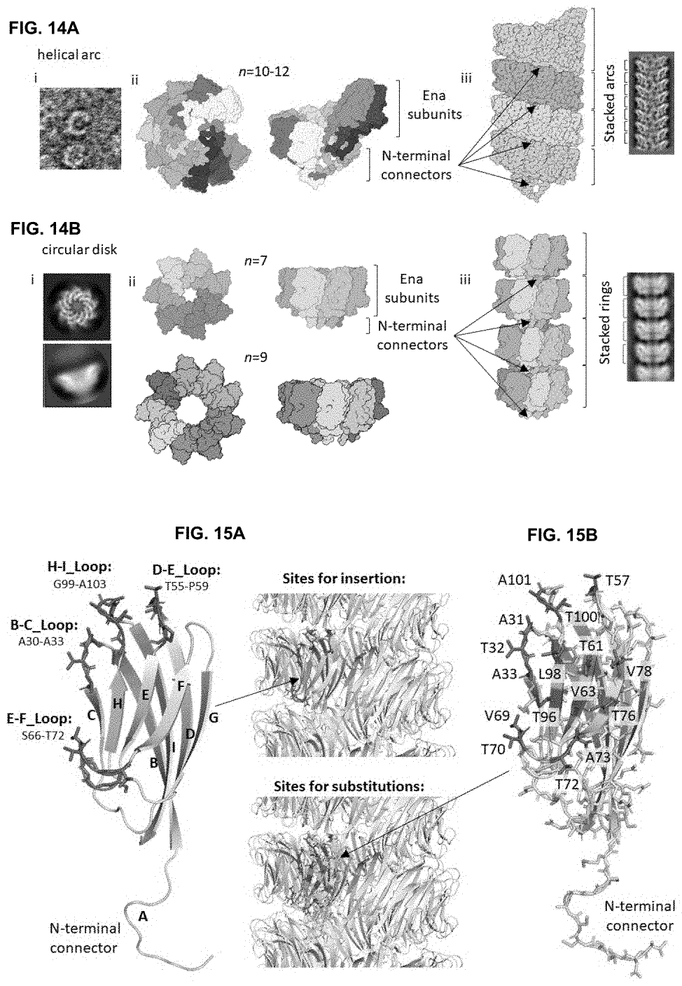

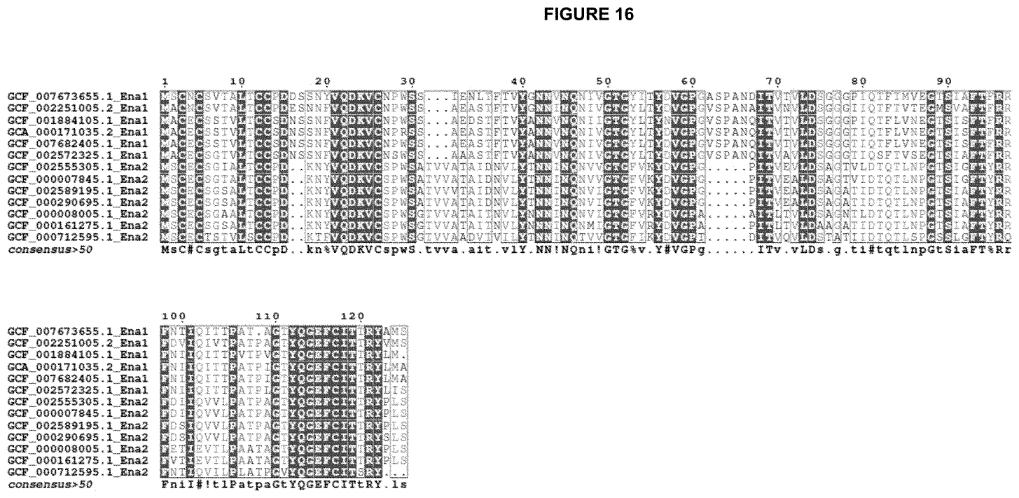

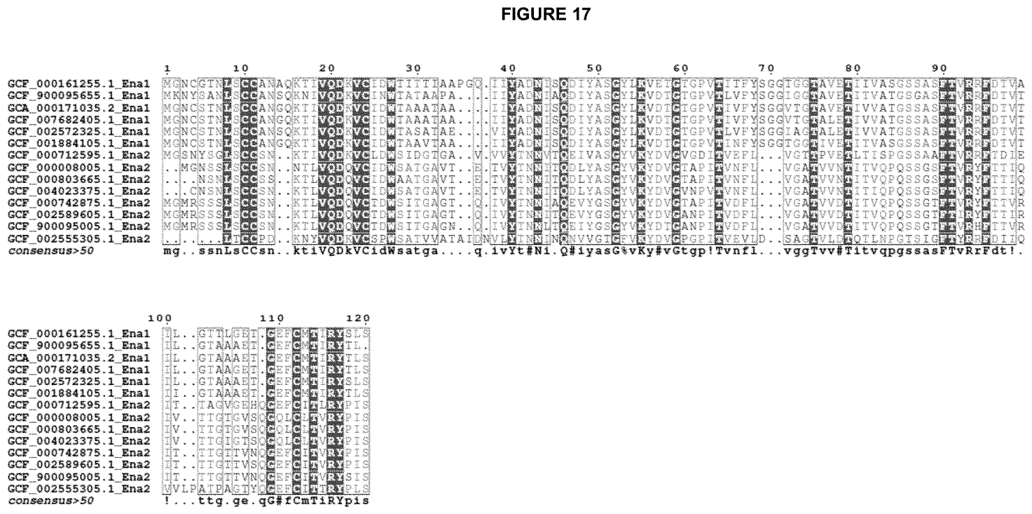

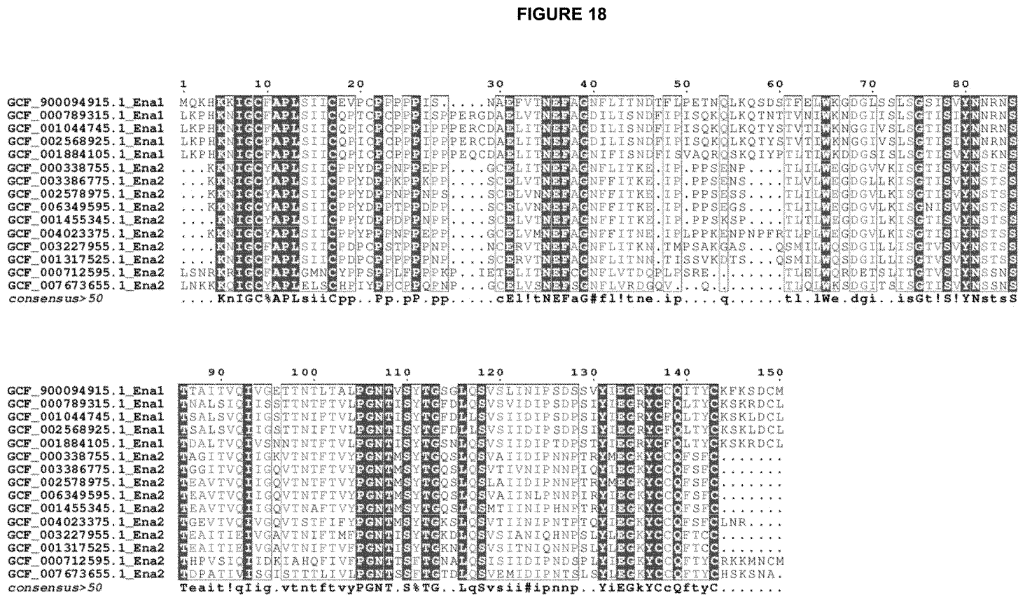

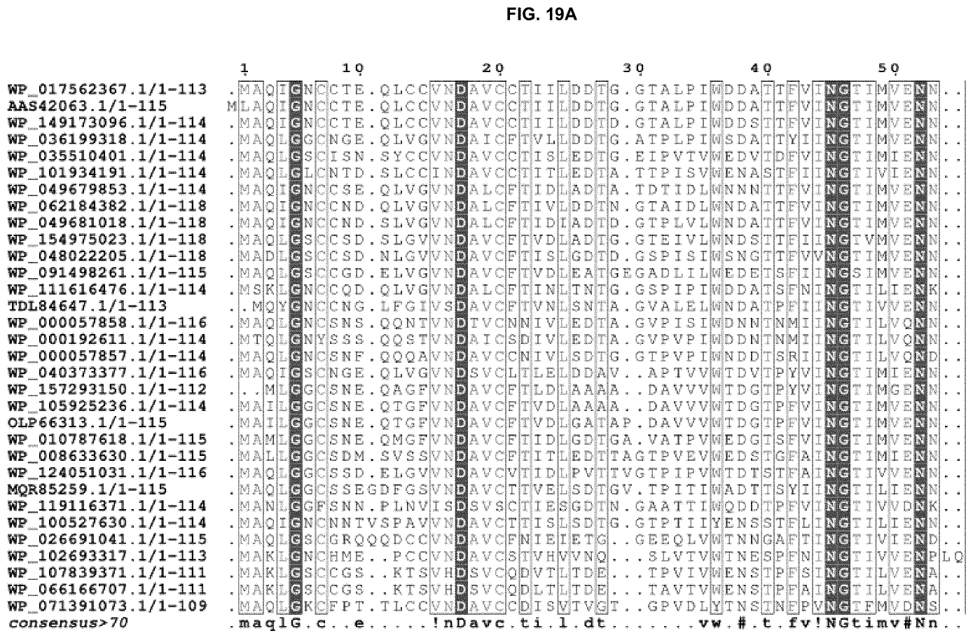

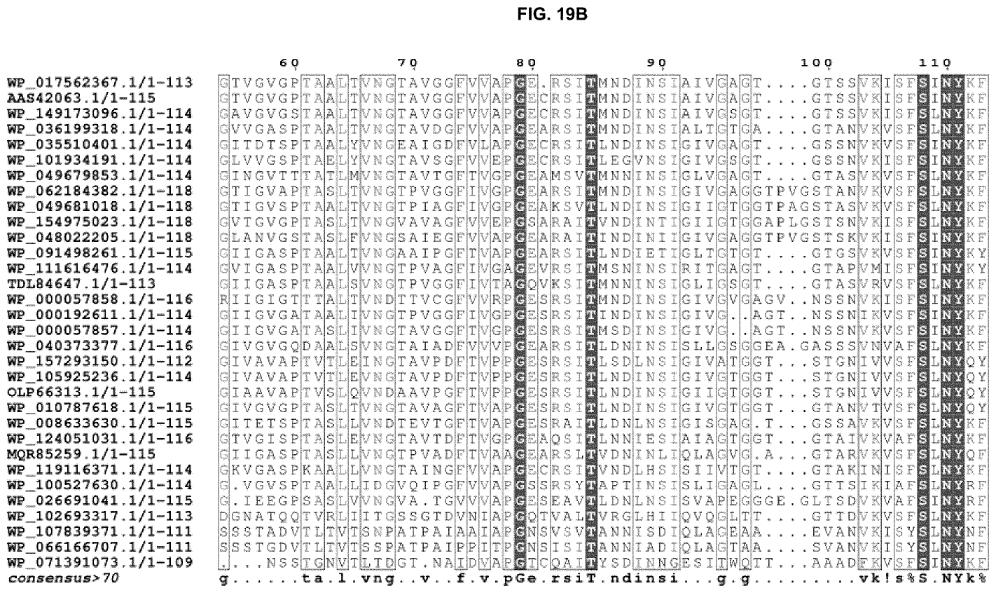

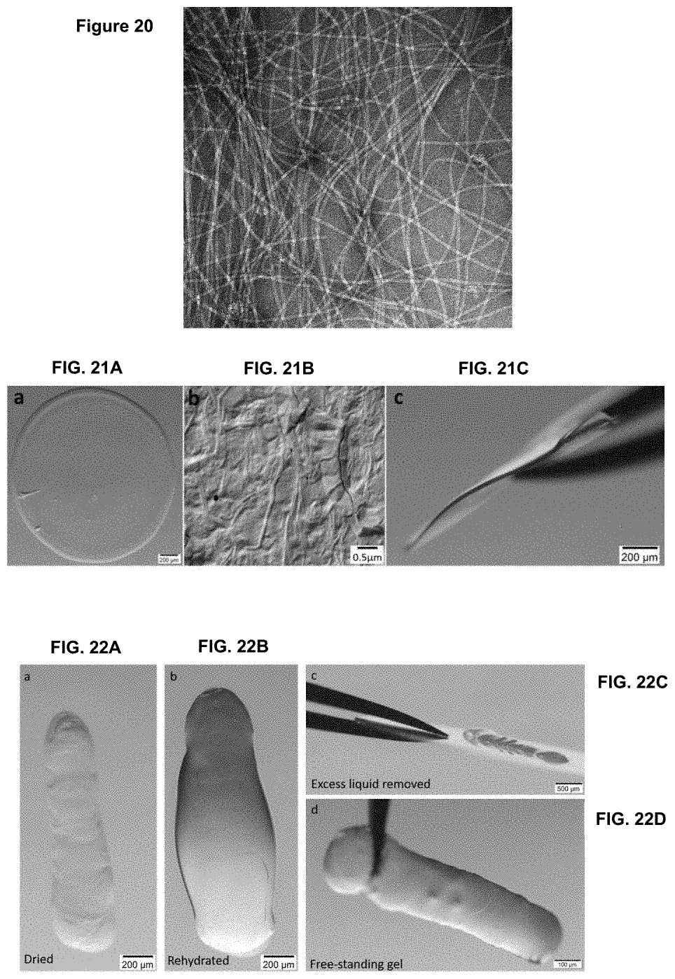

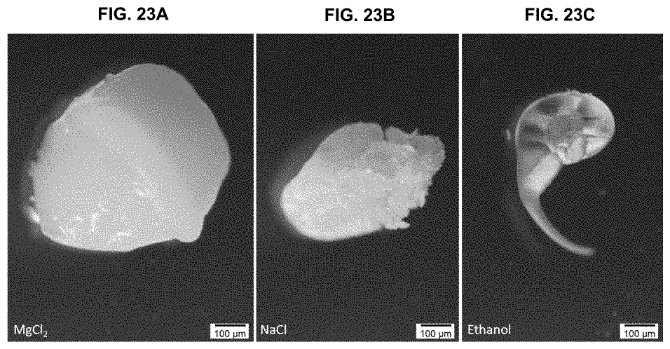

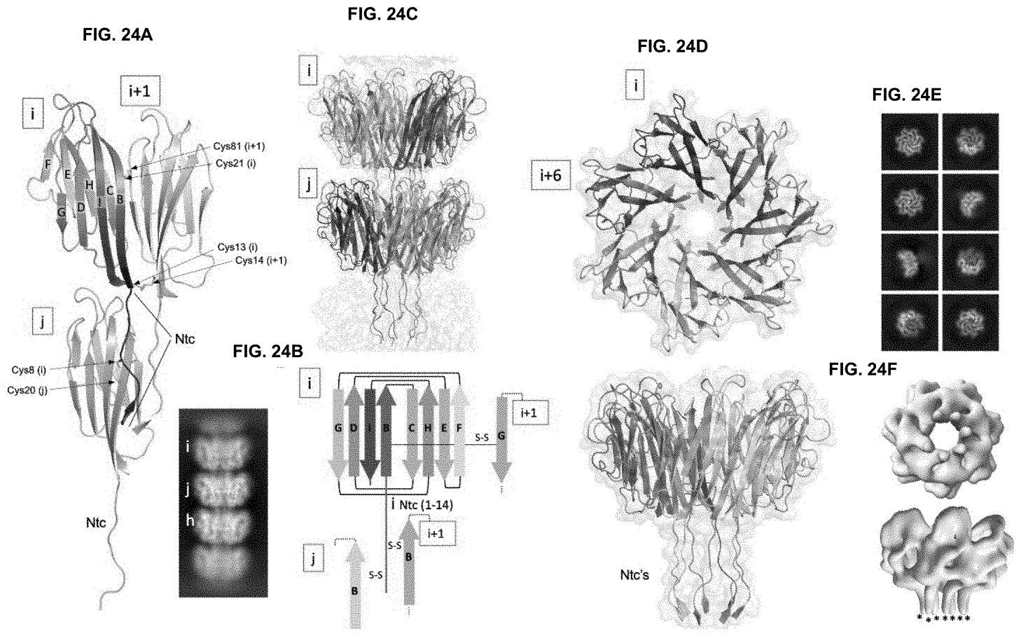

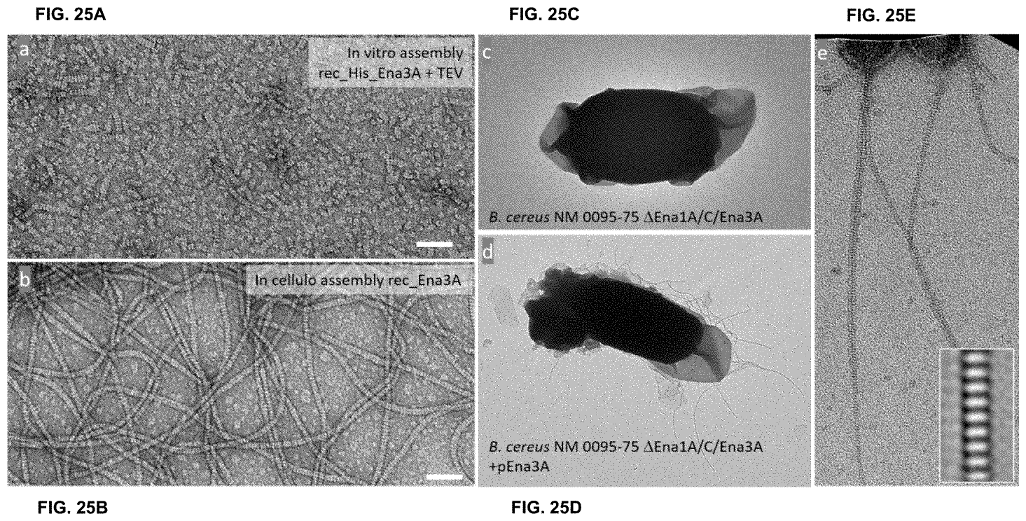

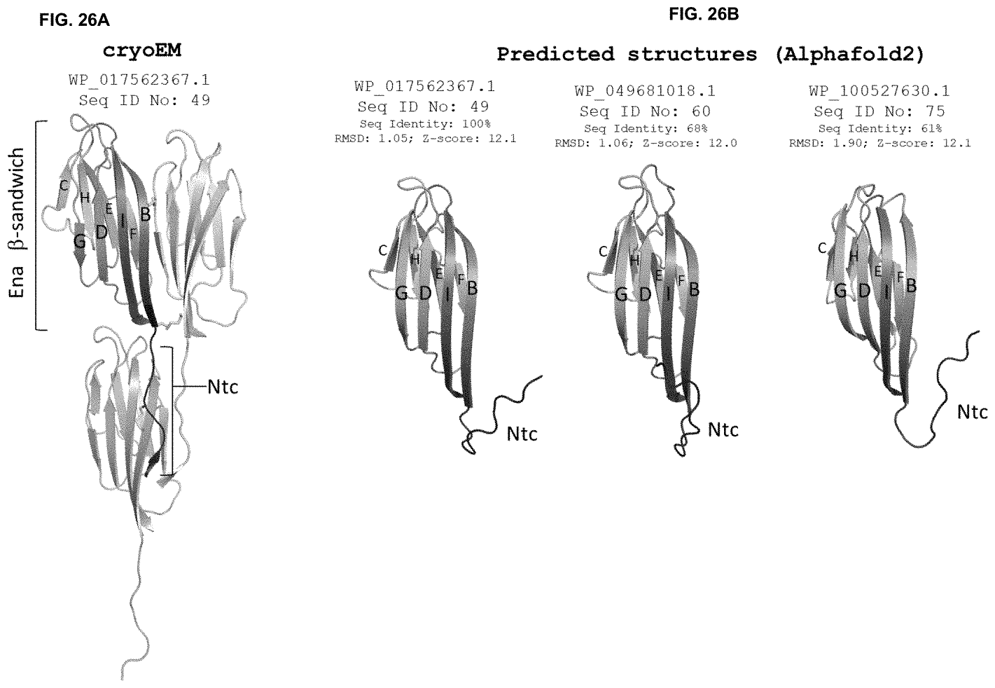

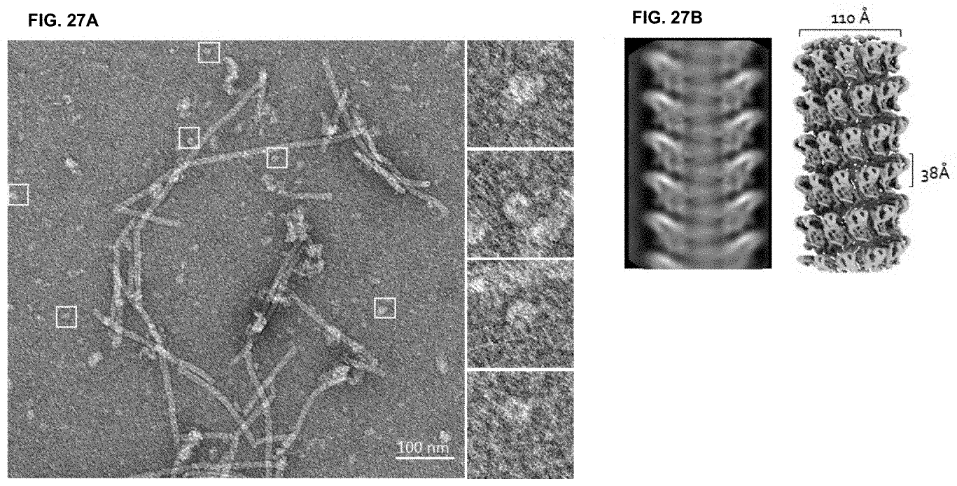

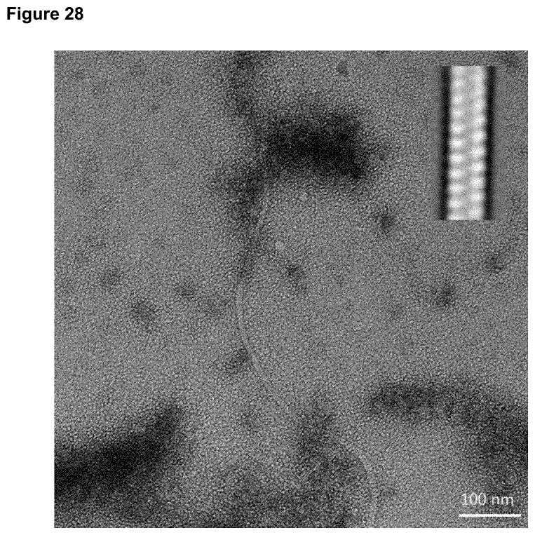

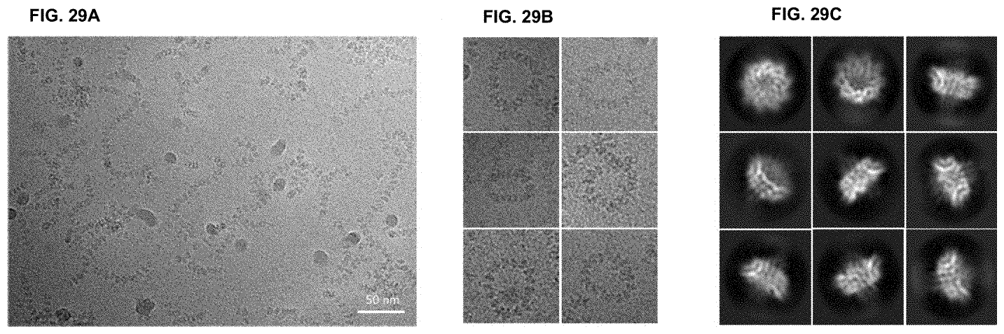

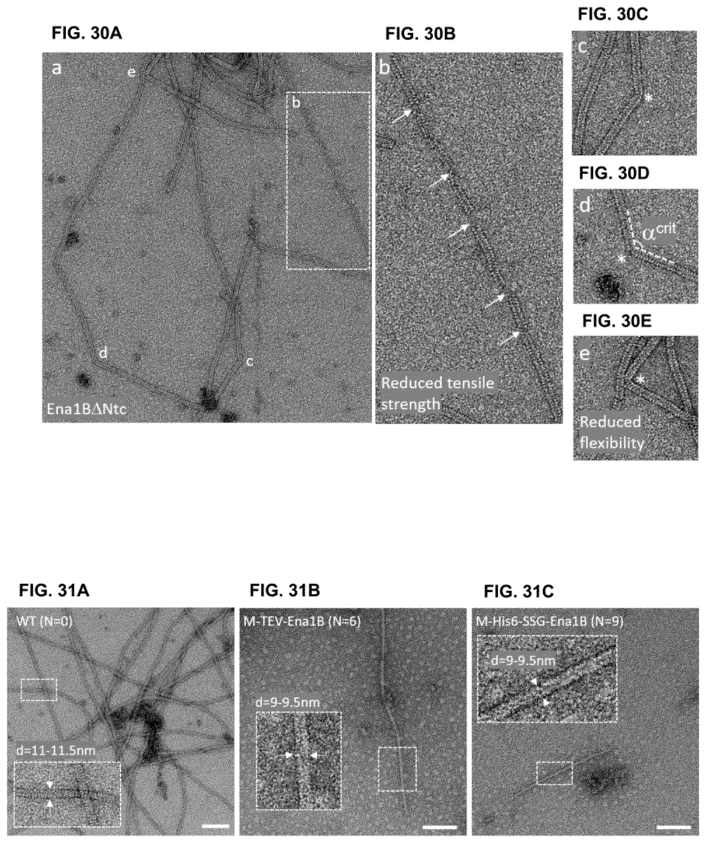

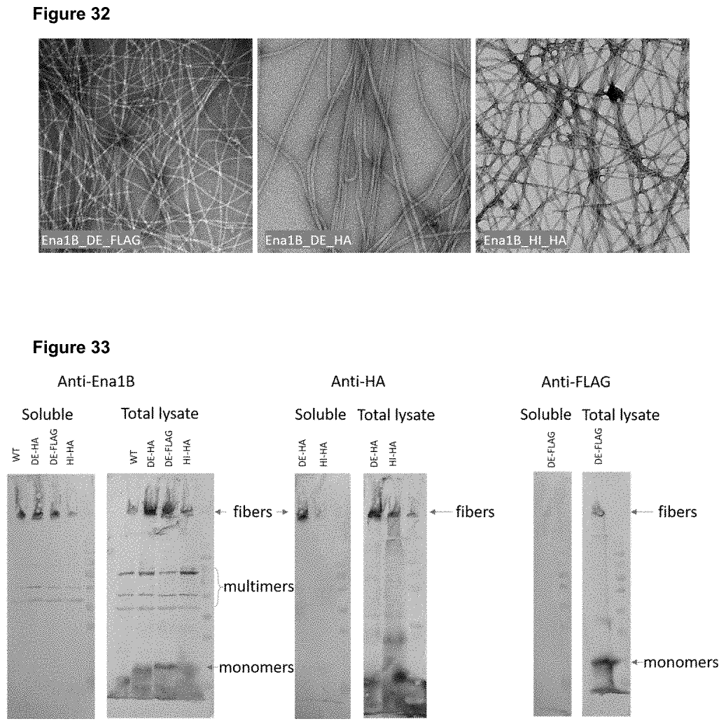

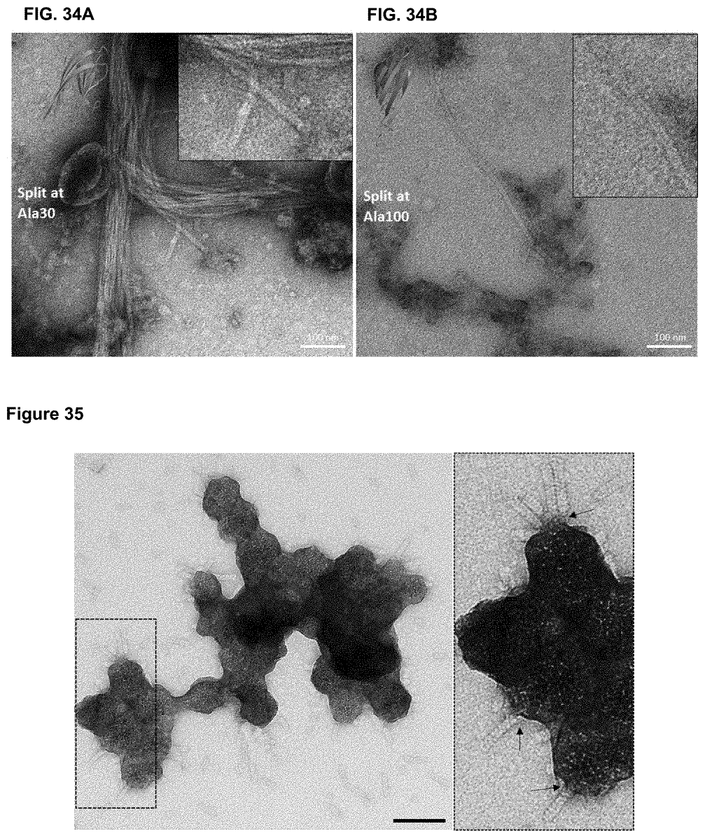

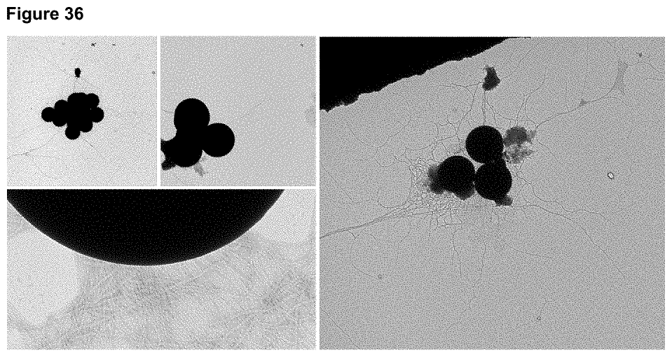

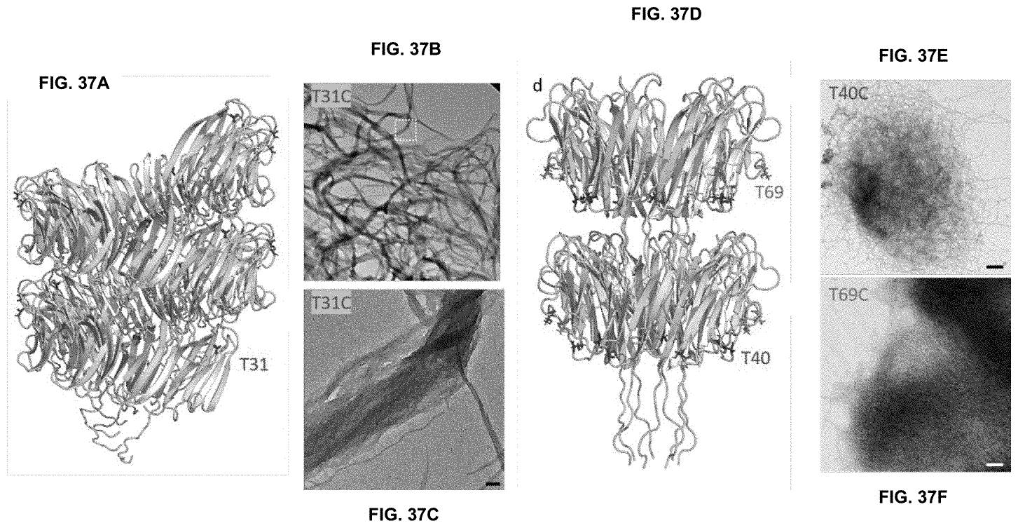

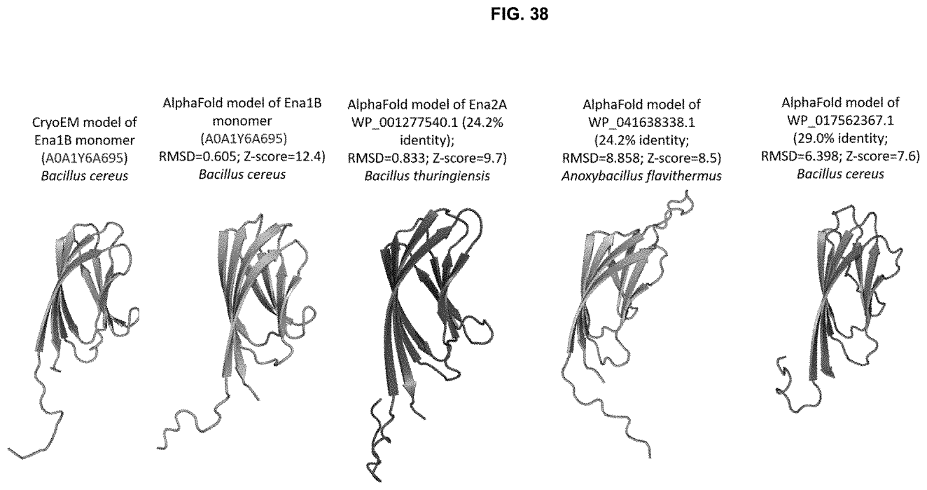

(FIGS. 2A and 2B) Representative 2D class average (FIG. 2A) and corresponding power spectrum (FIG. 2B) of B. cereus NVH 0075/95 S-type Enas viewed by cryoTEM. Bessel orders used to derive helical symmetry are indicated. (FIG. 2C) Reconstituted cryoEM electron potential map of ex vivo S-type Ena (3.2 Å resolution). (FIG. 2D) Side and top view of a single helical turn of the de novo built 3D model of S-type Ena shown in ribbon representation and molecular surface. Ena subunits are labelled i to i−10. (FIG. 2E) Ribbon representation and topology diagram of the S-type Ena1B subunit (blue to red rainbow from N- to C-terminus), and its interaction with subunits i−9 (sand) and i−10 (green) through disulphide crosslinking.

FIGS. 3A-3D. Ntc linkers give high flexibility and elasticity to S-type Enas.

(FIG. 3A) CryoTEM image of an isolated S-type Ena making a U-turn comprising just 19 helical turns (shown schematically in orange). (FIGS. 3B and 3C) Cross-section and 3D cryoTEM electron potential map of the S-type Ena model, highlighting the longitudinal spacing between Ena1B jellyroll domains as a result of the Ntc linker (residues 12-17). (FIG. 3D) Negative stain 2D class averages of endospore-associated S-type Enas show variation in pitch and axial curvature. These structural data on the recEna1B nanofiber identify the linker region as a site to engineer and modulate fiber rigidity and flexibility.

FIGS. 4A-4C. ena is bicistronic and expressed during sporulation.

(FIG. 4A) Chromosomal organization of the ena genes and primers used for transcript analysis (arrows). (FIG. 4B) Agarose gel electrophoresis (1%) analysis of PCR products using indicated primer pairs and cDNA made of mRNA isolated from NVH 0075/95 after 8 and 16 hrs growth in liquid cultures or genomic DNA as control. Of note, the expression of ena1C was surprisingly higher than ena1A and ena1B, who are components of the major appendages. (FIG. 4C) Transcription level of ena1A (x), ena1B (▴), ena1C(∘) and dedA (•) relative to rpoB determined by qRT-PCR during 16 hrs of growth of B. cereus strain NVH 0075/95. The dotted line represents the bacterial growth measured by increase, in OD₆₀₀. Whiskers represent standard deviation of three independent experiments.

FIGS. 5A and 5B. Composition of S- and L-type Ena.

(FIG. 5A) Representative negative stain images of endospores of NVH 0075/95 mutants lacking ena1A, ena1B, ena1A and B or ena1C, as well as the ena1B mutant complemented with ena1A-ena1B from plasmid (pAB). Inset are 2D class averages of Enas observed on the respective mutants. (FIG. 5B) Length distribution and number of Enas found on WT and mutant NVH 0075/95 endospores. Statistics: pair-wise Mann-Whitney U tests against WT (n: ≥18 spores; n: ≥50 Enas; ns: not significant, * p<0.05, ** p<0.01, *** p<0.001 and **** p<0.0001. ---: mean±s.d.)

FIGS. 6A and 6B. Ena is widespread in pathogenic Bacilli.

(FIG. 6A) Ena1 and Ena2 loci with average amino acid sequence identity indicated between the population of EnaA-C ortho- and homologues. Ena1C shows considerably more variation and is in B. cytotoxicus different from both Ena1C and Ena2C(see FIG. 11C), while other genomes have enaC located at a different loci (applies to two isolates of B. mycoides). (FIG. 6B) Distribution of ena1/2A-C among Bacillus species. Whole genome clustering of the B. cereus s.l. group and B. subtilis created by Mashtree (Katz et al., 2019; Ondov et al., 2016) and visualized in Microreact (Argimon et al., 2016). Rooted on B. subtilis. Traits for species (colored nodes), Bazinet clades and presence of ena are indicated on surrounding four rings in the following order from inner to outer: clades are annotated according to Bazinet 2017 (when available) (Bazinet, 2017), and presence of enaA, enaB and enaC (Ena1: teal, Ena2: orange, different locus: cyan). When no homo- or ortholog was found, the ring is grey. Ena1A-C and Ena2A-C are defined as ortho- or homologues when a protein is found in the corresponding genome having >90% coverage and >80% and 50-65% sequence identity, respectively, with Ena1A-C of the NMH 0095/75 strain. Interactive tree accessible at https://microreact.org/project/5UixxEY9vr2AVzXDVwa5t/8bcae82d.

FIGS. 7A-7C. Ena morphology and robustness.

(FIGS. 7A and 7B) Negative stain TEM of B. cereus NVH 0075/95 endospore with indication of the two Ena morphologies: S-type (black arrowheads) and L-type Enas (white arrowheads) (FIG. 7A), and closed-up view of a dislodged S-type Ena bundle splitting into individual Ena fibers (FIG. 7B). (FIG. 7C) Negative stain TEM images of isolated ex vivo S-type Ena. To test Ena stability under different stresses, samples were treated, from left to right, with: (1) untreated control, (2) 1 hour of 1 mg/ml proteinase K, (3) autoclaving (i.e. 20 min at 121° C.) or a 4 hour desiccation at 43° C. (4). Inset shows 2D class averages to assess the structural integrity of the treated Ena. S-type Ena are found to be resistant to Proteinase K treatment, autoclaving and desiccation at 43° C., although some fibers appear to lose subunit integrity upon desiccation (inset). Desiccation at 43° C. may mimic conditions encountered by Bacillus spores during drought.

FIGS. 8A-8F. S-type Ena structure determination and recombinant production.

(FIG. 8A) representative area of the 3D cryoEM potential map for ex vivo S-type Ena, at 3.2 Å resolution. An octameric peptide with sequence FCMTIRY (SEQ ID NO:88) was deduced de novo from the cryoEM potential map (shown in sticks) and used for a BLAST search of the B. cereus NVH 0075/95 genome. (FIG. 8B) Multiple sequence alignment of 3 ORF's (KMP91697.1: Ena1A SEQ ID NO: 1, KMP91698.1: Ena1B SEQ ID NO: 8 and KMP91699.1: Ena1C SEQ ID NO: 15) corresponding to DUF3992 containing proteins, of which the former two contain a sequence motif corresponding or similar to the one deduced from the EM potential map (shaded in cyan). The three ORFs are here shown to correspond to the S-type Ena subunits (see main text) and are hereafter referred to as Ena1A, Ena1B and Ena1C, respectively. Secondary structure and structural elements as determined from the built model (see FIG. 2) are shown schematically above the sequences (Ntc: N-terminal connecter; arrows correspond to β-strands, labelled as in FIG. 2). (FIG. 8C) SDS PAGE of recombinant Ena1B, expressed in E. coli, affinity purified under denaturing conditions (8M urea) and treated with β-mercaptoethanol or TEV protease (to remove N-terminal 6-His tag) as indicated. TEV cleavage results in a species of apparent MW 12.1 KDa, corresponding to the expected MW of the Ena1B monomer. (FIG. 8D) Negative stain TEM images of rec1Ena1B oligomers formed after refolding. (FIG. 8E) Closed up view that shows recEna1B oligomers form open crescents similar in dimensions and shape to single helical turns or arcs found in the S-type Ena fiber (model—right). Steric hindrance by the N-terminal His-tag is thought to arrest recEna1B polymerization into single helical arcs. (FIG. 8F) Negative stain image and 2D classification of Ena-like fibers formed after TEV digestion of recEna1B. Upon removal of the N-terminal His-tag, recEna1B readily assembles into fibers with helical properties closely resembling those found for ex vivo S-type Enas.

FIGS. 9A-9E. Native S-type Ena are composed of both Ena1A and Ena1B subunits.

(FIG. 9A) FSC curve and local resolution heatmap (inset) of the recEna1B helical reconstruction, indicating a final resolution of 3.2 Å at a cutoff of 0.143. FSC curve and local resolution were calculated by postprocessing in RELION3.0 using a solvent mask consisting of 3 helical turns. (FIGS. 9B and 9C) Side-by-side comparison of cryoEM maps calculated from of ex vivo (FIG. 9B) and recENA1B filaments (FIG. 9C), with the refined Ena1B model docked into the maps. The ex vivo Ena map shows features unaccounted for by the Ena1B model near loops 3 (L3) and 7 (L7), corresponding to regions of amino acid insertions in the Ena1A sequence (FIG. 8B). (FIG. 9D) recEna1B map (pink) and recEna1B-ex vivo difference map (green) masked over a single Ena1B subunit and calculated by TEMPy:Diffmap (Farabella et al., 2015) from the CCPEM package (Burnley et al., 2017). Difference in both maps locate to L3, L7 and the conformation of Ntc. (FIG. 9E) Immunogold TEM of ex vivo S-type Ena, stained with, from left to right, anti-Ena1A, anti-Ena1B and anti-Ena1C sera, each with gold-labeled (10 nm) anti-rabbit IgG as secondary antibody. Specific staining with Ena1A and Ena1B sera confirm the presence of both subunits in native Ena. No staining was seen with Ena1C serum.

FIGS. 10A-10D. Inter-subunit interactions in S-type Ena.

(FIGS. 10A and 10B) Ribbon (FIG. 10A) and schematic (FIG. 10B) representation of lateral subunit—subunit contacts in S-type Ena. Strand G of BIDG sheet of each subunit is augmented with strand C of CHEF β-sheet of the succeeding subunit. Both subunits are covalently cross-linked via the Ntc (blue) of a subunit located, respectively, 9 or 10 subunits above. Cys11 and Cys10 go into a disulphide bond with residues 24 in the B strand of subunit i−10 and Cys109 in strand I of subunit i−9. (FIGS. 10C and 10D) Coulomb potential maps (calculated in PyMOL) of two adjacent subunits (FIG. 10C) and two helical turns of the S-type Ena showing the distribution of charge on the atomic model surface. Each subunit possesses complementary positive and negatively charged patches of residues at the inter-subunit surface that are responsible for electrostatic stabilizing interactions between the subunits. Similarly, stacked helical rings in the S-type Ena show a charge complementary interface (FIG. 10D).

FIGS. 11A-11C. Phylogenetic relationship between EnaA-C protein sequences among Bacillus spp.

Approximate likelihood trees generated by FastTree v.2.1.8 (Price et al., 2010), visualized in Microreact (Argimon et al., 2016). Trees are rooted on midpoint. Nodes are colored according to annotated species. See Methods for further details. (FIG. 11A) Relationship between Ena1A and Ena2A isoforms of 593 isolates. Ena1A and Ena2A are defined as ortho- or homologues having >90% coverage and >80% and 50-65% sequence identity, respectively, with Ena1A_GCF_001044825; KMP91697.1 protein sequence defined in SEQ ID NO: 1. Interactive tree accessible at https://microreact.org/project/5UixxEY9vr2AVzXDVwa5t/1a8558fd. FIG. 11B) Relationship between Ena1B and Ena2B isoforms of 591 isolates. Ena1B, Ena1B_candidate and Ena2B are defined as ortho- or homologues with >90% coverage and >80%, 60-80% and 40-60% sequence identity to Ena1B_NM_Oslo protein sequence defined in SEQ ID NO:87, respectively. Interactive tree accessible at https://microreact.org/project/iJ4pARvgf9gyT916sTar5u/1332f3b3. (FIG. 11C) Relationship between Ena1C and Ena2C isoforms of 591 isolates. Ena1C, Ena1C_candidate and Ena2C_candidate are defined as ortho- or homologues with >90% coverage and >80%, 60-80% and 40-60% sequence identity to Ena1C protein sequence defined in SEQ ID NO:15 (KMP91699.1), respectively. Furthermore, isolates in which an ortho- or homologue was found elsewhere in the genome than the usual EnaA-B locus are coloured cyan. Isolates that lacked an Ena1C homo- or orthologue are colored grey. Interactive tree accessible at https//:microreact.org/project/aQaqCUCJoj2mw55KQujbGY/0990885.

FIG. 12: In vivo recombinantly produced Ena1A S-type fibers.

60k magnification TEM image of negatively stained Ena1A fibers that were formed in the cytoplasm of E. coli following recombinant expression of monomeric subunits.

FIGS. 13A and 13B. Schematic representation of the Ena building blocks for self-assembly.

(FIG. 13A) S-type fibers: monomeric Ena1/2 subunits with N-terminal connectors harboring a steric block, self-assemble in vitro into a multimeric, helical arrangement but are hindered to form higher order structures. Multimers in this arrangement are comprised of 10 to 12 monomers. Removal of steric blocks (via proteolytic cleavage) triggers stacking of multimers in a head-to-tail configuration and/or incorporation of monomeric entities at either terminus, giving rise to a helical, fibrous assembly of indefinite size.

(FIG. 13B) L-type fibers: monomeric Ena3A or Ena1C subunits with N-terminal connectors harboring a steric block self-assemble in vitro into a multimeric, circular arrangement but are hindered to form higher order structures. Multimers in this arrangement are comprised of 7 to 9 monomers. Removal of steric blocks (via proteolytic cleavage) of Ena3A multimers triggers stacking of said multimers in a head-to-tail configuration giving rise to a cylindrical, fibrous assembly of indefinite size.

FIGS. 14A and 14B. Detailed structural composition of Ena multimeric and fibrous assemblies.

(FIG. 14A) Helical arc multimers and S-type fibers: (left-i) top NS-EM class average of a helical Ena multimer; (middle-ii) top and side-view of helical Ena arc arrangements derived from in vitro produced recEna1B cryoEM volumes: Ena monomers are colored separately; (right-iii) helical S-type fiber composed of head-to-tail stacked Ena arcs interlocking via N-terminal connectors that interface with the C-terminal receiver regions of the adjacent arc.

(FIG. 14B) Circular disk multimers and L-type fibers: (left-i) top and side-view cryo-EM class averages of in vitro produced nonameric Ena1C multimers; (middle-ii) top and side-view of heptameric Ena3A multimers, and nonameric Ena1C ring arrangements derived from cryoEM volumes: Ena monomers or subunits are colored separately; (right-iii) heptameric L-type fiber composed of head-to-tail stacked Ena3A heptameric rings interlocking via N-terminal connectors that interface with the C-terminal receiver regions of the adjacent ring.

FIGS. 15A and 15B. Ena1B nanofiber engineering sites.

The recEna1B (SEQ ID NO:84) structure is used here to demonstrate the suitable sites for insertion of single amino acids, peptides or full domains into loops connecting strands E-F, B-C, H-I and D-E (FIG. 15A), or Sites for single-site substitutions (FIG. 15B; highlighted in red).

FIG. 16. Multiple sequence alignment of Ena1/2A protein sequences.

The identifiers correspond to SEQ ID NOs: 1-7 for Ena1A and SEQ ID NOs: 21-28 for Ena2A.

FIG. 17. Multiple sequence alignment of Ena1/2B protein sequences.

The identifiers correspond to SEQ ID NOs: 8-14 for Ena1B and SEQ ID NOs: 29-37 for Ena2B.

FIG. 18. Multiple sequence alignment of Ena1/2C protein sequences.

The identifiers correspond to SEQ ID NOs: 15-20 for Ena1C and SEQ ID NOs:38-48 for Ena2C

FIGS. 19A and 19B. Multiple sequence alignment of Ena3 protein sequences.

Multiple sequence alignment of selected, representative Ena3 homologues, corresponding to SEQ ID NOs: 49-80.

FIG. 20. Negative stain transmission electron micrograph of recombinant Ena1B S-type fibers.

3 μl of a 1 mg·mL⁻¹ Ena1B suspension was deposited onto a Cu-mesh formvar grid, washed 3× in miliQ followed by 1% (w/v) uranyl acetate.

FIGS. 21A-21C: A thin film produced from Ena1B S-type fibers.

(FIG. 21A) Translucent Ena1B S-type thin film on a siliconized cover slip, (FIG. 21B) top and (FIG. 21C) side view of a free-standing Ena1B S-type thin film dislodged from a siliconized cover slip after drop-casting a 100 mg·mL⁻¹ Ena1B S-type solution. Estimated thickness is 21 μm.

FIGS. 22A-22D: A soft hydrogel from Ena1B S-type fibers.

(FIG. 22A) Translucent Ena1B S-type thin film on a siliconized cover slip, (FIG. 22B) rehydration step through application of 50 μl miliQ, (FIG. 22C) side view of resulting hydrogel after removal of excess miliQ water, (FIG. 22D) free-standing, translucent, Ena hydrogel gripped between tweezers.

FIGS. 23A-23C. Reinforced Ena hydrogel beads after dehydration in 4M MgCl₂ (FIG. 23A), 5M NaCl (FIG. 23A) and 100% (v/v) Ethanol (FIG. 23C).

FIGS. 24A-24F. 1-type fibers constituted of Ena3A proteins.

(FIG. 24A) Ribbon and (FIG. 24B) schematic representation of lateral (i/i+1) and axial (i/j) subunit—subunit contacts in L-type Ena. Inter-ring crosslinking is established via the N-terminal connector (Ntc) which forms a disulphide bond at position Cys8 (i) with Cys20 of subunit j in the neighbouring ring; lower inset: cryoEM 2D class average of the L-type fibers; (FIG. 24C) Cartoon representation of two heptameric Ena3A rings that were built into the 3.5 Å cryoEM map (transparent volume in white); (FIG. 24D) Top and side view of a model of a single Ena3A heptamer; (FIG. 24E) cryoEM 2D class averages of sterically blocked 6×His_TEV_Ena3A multimers and (FIG. 24F) corresponding cryoEM volume.

FIGS. 25A-25E. Ena3A is essential and sufficient for L-type fiber production.

(FIG. 25A) In vitro assembly of short L-type fibers obtained from purified, sterically blocked Ena3A multimers after co-incubation with TEV protease; (FIG. 25B) In cellulo assembly of long L-type Ena3A fibers after recombinant expression of WT recEna3A in E. coli and subsequent isolation of the fiber fraction; (FIG. 25C) nsTEM image of a mature spore from a quadruple Ena-knockout strain (Δena1A-1B-1C-ena3A) derived from B. cereus NM 0095-75: representative image demonstrating complete absence of any endospore appendages; (FIG. 25D) nsTEM image of the quadruple Ena-knockout strain transformed with pENA3A: phenotypic rescue of L-type fibers on the spore surface; (FIG. 25E) Zoom-in image of the L-type Ena3A fibers on the surface of the rescue strain shown in (FIG. 25D) with corresponding 2D class in bottom inset confirming L-type morphology.

FIGS. 26A and 26B. Structural comparison of a number of selected Ena3A homologues.

(FIG. 26A) CryoEM structure of the Ena3A L-type Ena fiber of Bacillus cereus strain ATCC_10987 (WP_017562367.1; SEQ ID NO:49) showing three subunits to document lateral and longitudinal contacts in the fiber. Ena subunits are defined by an 8-stranded β-sandwich fold with a BIDG-CHEF topology, as well as an N-terminal extension peptide referred to as the Ntc, and responsible for the longitudinal covalent contacts in the fibers (FIG. 19). (FIG. 26B) Predicted structures of selected Ena3A homologues. For each structure, we provide the root-mean-square-deviation (RMSD) of atomic positions between Cα atom i of each structure and the corresponding Cα atom of the reference structure (cryoEM model of Ena3A: WP_017562367.1, SEQ ID NO: 49), as well as the fold similarity score, i.e. the Dali Z-score. For WP_049681018.1 (SEQ ID NO: 60) and WP_100527630.1 (SEQ ID NO:75), we provide the putative structures as predicted by AlphaFold v2.0. As a benchmark, we also provide the AlphaFold model of our reference structure Ena3A (WP_017562367.1), demonstrating excellent agreement between the experimental cryoEM structure and the AlphaFold model (RMSD=1.05; Z=12.1).

FIGS. 27A and 27B. In vitro assembly of Ena2A into S-type fibers.

FIG. 27A) NS-TEM micrographs of Ena2A filaments recombinantly expressed in E. coli B121 DE3 pLysS with N-terminal 6×His blocker then assembled in vitro after removal of the blocker by cleavage using TEV protease. Squares highlighting Ena2A multimer spirals (diameter ˜10 nm) resulting from incomplete removal of the N-terminal blocker, on right, zoomed in micrograph crop-outs of individual multimers. FIG. 27B) Cryo-EM 2D class average of in vitro assembled Ena2A filament showing higher resolution features that look like earlier obtained 2D class averages of Ena1B. On right, Snapshot of 3D reconstruction volume (resolution=5 Å) of Ena2A filament with pitch ˜38 Å and diameter 110 Å generated by helical reconstruction with helical parameters of twist=31.01 degrees and rise=3.15 Å.

FIG. 28.1n cellulo assembly of Ena2A into S-type fibers.

NS-TEM image of Ena2A recombinantly expressed in B121 (DE3) C43 E. coli without any N-terminal blocker, top right negative stain 2D class average confirming the identity of S-Ena fibers.

FIGS. 29A-29C. Ena2C assembled into nonameric discs and short L-like filaments in vitro.

FIG. 29A) Cryo-EM 2D micrographs of short L-like Ena2C filaments recombinantly expressed in E. coli Bl21 C43 with N-terminal 6×His blocker then assembled in vitro after removal of the blocker by cleavage using TEV protease. The resulting filaments are highly flexible and curve to form closed loops. FIG. 29B) Cryo-EM 2D micrograph crop-outs of Ena2C L-like filament closed loops of approximated diameter 70 nm containing 15-20 Ena2C nonameric discs. FIG. 29C) Cryo-EM 2D Class averages of Ena2C nonameric discs displaying various orientations of the multimer.

FIGS. 30A-30E. Impact of the Ntc deletion on the Ena1B S-type fiber strength and flexibility.

Recombinant Ena1BΔNtc fibers present in the extracellular milieu (FIG. 30A), exhibiting rupture (FIG. 30B) and fracture points (FIGS. 30C-30E) as a result of reduced tensile strength and flexibility.

FIGS. 31A-31C. Impact of the length of the steric block on the ability of Ena1B to self-assemble into S-type fibers, monitored via ns-TEM.

(FIG. 31A) WT Ena1B S-type fibers—no steric block (N=0); (FIG. 31B) M-TEV-Ena1B (N=6); (FIG. 31C) M-His6-SSG-Ena1B (N=9). Scalebar represents 100 nm.

FIG. 32. Demonstration of the engineerability of Ena1B loops with respect to peptide tag insertion.

Examples shown for loops DE and HI (as indicated in FIG. 15), and inserts of linear tags FLAG and HA.

FIG. 33. Western blot analysis of WT Ena1B and various loop-modified Ena1B constructs (DE-HA, DE-FLAG, HI-HA) using a-Ena1B, a-HA and a-FLAG primary antibodies.

All 4 constructs (SEQ ID NO:8 for WT Ena1B, and SEQ ID NOs: 140-142 for Ena1B insertion variants) were expressed in E. coli after which total cell lysates and soluble fractions were loaded onto SDS-PAGE. Anti-Ena1B panel: high molecular weight bands of Ena1B that are retained in the stacking gel correspond to SDS-insoluble fibers (see nsTEM images in FIG. 32); Anti-HA and anti-FLAG panels: Fiber fractions of DE-HA, HI-HA and DE-FLAG stain positive against a-HA and a-FLAG, respectively, demonstrating surface accessibility of the peptide tags when Ena1B is assembled into the fiber ultrastructure.

FIGS. 34A and 34B. Ena1B assembles into S-type Ena fibers in cellulo, upon co-expression of split Ena constructs.

The split of Ena1B in the BC or HI loop at Ala30 or Ala100, respectively. FIG. 34A) NS-TEM micrograph of split-BC Ena1B S-Ena. Top left cartoon representation of split Ena1B structure highlighting the split halves namely strands AB (in orange) and strands CDEFGHI (in green). Top right box, cropped and zoomed image confirming the presence of S-Ena filaments. FIG. 34B) NS-TEM micrograph of split-HI Ena1B S-Ena. Top left cartoon representation of split Ena1B structure highlighting the split halves namely strand I (in magenta) and strands ABCDEFGH (in green). Top right box, cropped and zoomed image confirming the presence of S-Ena filaments.

FIG. 35. Epitaxial growth of S-type fibers on solid supports.

Scalebar represents 100 nm.

FIG. 36. non-covalent Ena fiber functionalization of solid surfaces.

nsTEM analysis micrograph of biotinylated Ena1B S-type fibers on streptavidin-coated gold beads.

FIGS. 37A-37F. Engineering of Ena proteins by site-directed mutagenesis to modify Ena fiber networks.

Site-directed mutagenesis sites for Ena1B S-type fibers: surface exposed residues T31 was selected for mutagenesis into a cysteine residue (FIG. 37A); corresponding ns-TEM images of ex vivo purified fibers recombinantly expressed in E. coli of Ena1B T31C (FIG. 37B) and zoom-in corresponding to the dashed white box. (FIG. 37C); site-directed mutagenesis sites for Ena3A L-type fibers: surface exposed residues T40 and T69 were selected for mutagenesis into a cysteine residue (FIG. 37D); corresponding ns-TEM images of ex vivo purified fibers recombinantly expressed in E. coli Ena3AT40C and Ena3AT69C. Scalebars correspond to 100 nm (FIG. 37C) or 200 nm (FIGS. 37E and 37F). Cross-linked Ena fibers assemble into reinforced bundles or ‘ropes’, and clustered hydrogels.

FIG. 38. Structural comparison of a number of selected Ena homologues using Alpha fold prediction.

Cryo-EM structure for Ena1B (UniProt. A0A1Y6A695) was compared with the Alphafold predicted fold structures for Ena1B itself, and the predicted Ena2A (NCBI ID: WP_001277540.1; SEQ ID NO:145), WP_017562367.1 and WP_041638338.1 protein sequences. RMSD, root-mean-square-deviation of atomic positions between atom i of each structure and the corresponding atom of the reference structure (cryoEM model of Ena1B—Uniprot: A0A1Y6A695; corresponding to SEQ ID NO:8), as well as the fold similarity score, i.e. the Dali Z-score (Jumper et al., 2021 Nature; doi.org/10.1038/s41586-021-03819-2).

### DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The term “nucleic acid sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. By “nucleic acid construct” it is meant a nucleic acid molecule that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into m RNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. “Promoter region of a gene” or “regulatory element” as used here refers to a functional DNA sequence unit that, when operably linked to a coding sequence and possibly placed in the appropriate inducing conditions, is sufficient to promote transcription of said coding sequence. “Operably linked” refers to a juxtaposition wherein the components so described are in a relationship permitting them to function in their intended manner. A promoter sequence “operably linked” to a nucleic acid molecule that is a coding sequence is ligated in such a way that expression of the coding sequence is achieved under conditions compatible with the promoter sequence. “Gene” as used here includes both the promoter region of the gene as well as the coding sequence. It refers both to the genomic sequence (including possible introns) as well as to the cDNA derived from the spliced messenger, operably linked to a promoter sequence. The term “terminator” or “transcription termination signal” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another gene. With a “chimeric gene” or “chimeric construct” or “chimeric gene construct” is meant a recombinant nucleic acid sequence molecule in which a promoter or regulatory nucleic acid sequence is operatively linked to, or associated with, a nucleic acid sequence that codes for an mRNA, such that the promoter or regulatory nucleic acid sequence is able to regulate transcription or expression of the associated nucleic acid coding sequence. The regulatory nucleic acid sequence of the chimeric gene is not operatively linked to the associated nucleic acid sequence as found in nature, and may be heterologous to the encoding nucleic acid sequence molecule, meaning that its sequence is not present in nature in the same constellation as presented in the chimeric construct. More general, the term “heterologous” is defined herein as a sequence or molecule that is different in its origin.

The terms “protein”, “polypeptide”, and “peptide” are interchangeably used further herein to refer to a polymer of amino acid residues and to variants and synthetic analogues of the same. A monomeric or protomer is defined as a single polypeptide chain from amino-terminal to carboxy-terminal ends. A “protein subunit” as used herein refers to a monomer or protomer, which may form part of a multimeric protein complex or assembly.

The terms “chimeric polypeptide”, “chimeric protein”, “chimer”, “fusion polypeptide”, “fusion protein”, are used interchangeably herein and refer to a protein that comprises at least two separate and distinct polypeptide components that may or may not originate from the same protein. The term also refers to a non-naturally occurring molecule which means that it is man-made. The term “fused to”, and other grammatical equivalents, such as “covalently linked”, “connected”, “attached”, “ligated”, “conjugated” when referring to a chimeric polypeptide (as defined herein) refers to any chemical or recombinant mechanism for linking two or more polypeptide components. The fusion of the two or more polypeptide components may be a direct fusion of the sequences or it may be an indirect fusion, e.g. with intervening amino acid sequences or linker sequences, or chemical linkers. The fusion of amino acid residues or (poly)peptides to an Ena protein or to another protein of interest as described herein, may be a covalent peptide bond, or also refer to a fusion obtained by chemical linking. The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link.

The term “molecular complex” or “complex” refers to a molecule associated with at least one other molecule, which may be a protein or a chemical entity. The term “associating with” refers to a condition of proximity between a chemical entity or compound, or portions thereof, and a binding pocket or binding site on a protein. As used herein, the term “protein complex” or “protein assembly” or “multimer” refers to a group of two or more associated macromolecules, whereby at least one of the macromolecules is a protein. A protein complex or assembly, as used herein, typically refers to binding or associations of macromolecules that can be formed under physiological conditions. Individual members of a protein complex, such as protein subunits or protomers, are linked by non-covalent or covalent interactions. “Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. The binding or association maybe non-covalent—wherein the juxtaposition is energetically favoured by for instance hydrogen bonding or van der Waals or electrostatic interactions—or it may be covalent, for instance by peptide or disulphide bonds.

It will be understood that a protein complex can be multimeric. Protein complex assembly can result in the formation of homo-multimeric or hetero-multimeric complexes. Moreover, interactions can be stable or transient. The term “multimer(s)”, “multimeric complex”, or “multimeric protein(s) or assemblies” comprises a plurality of identical or heterologous polypeptide monomers. Polypeptides can be capable of self-assembling into multimeric assemblies (i.e.: dimers, trimers, pentamers, hexamers, heptamers, octamers, etc.) formed from self-assembly of a plurality of a single polypeptide monomers (i.e., “homo-multimeric assemblies”) or from self-assembly of a plurality of different polypeptide monomers (i.e. “hetero-multimeric assemblies”). As used herein, a “plurality” means 2 or more. The multimeric assembly comprises 3, 4, 5, 6, 7, 8, 9, 10, 11, 12, or more polypeptide monomers. The multimeric assemblies can be used for any purpose and provide a way to develop a wide array of protein “nanomaterials.” In addition to the finite, cage-like or shell-like protein assemblies, they may be designed by choosing an appropriate target symmetric architecture. The monomers or protomers and/or multimeric assemblies of the invention can be used in the design of higher order assemblies, such as fibers, with the attendant advantages of hierarchical assembly. The resulting multimeric or fibrous assemblies are highly ordered materials with superior rigidity and monodispersity, and can be functional as a multimer or fiber itself, or form the basis of advanced functional materials, such as modified surfaces containing multimeric assemblies or fibers, and custom-designed molecular machines with wide-ranging applications. More specifically, a multimer as used herein refers to homo- or heteromultimeric protein complexes which are non-covalently associated with each other to form an arc, turn, ring or disc-like structure; and/or further modified to grow or develop into self-assembling or triggered formation of nanofibers. Said multimeric assemblies may contain Ena proteins as defined herein, or Ena protein variants, mutant and/or engineered Ena proteins, as well as other proteins that may associate to said Ena protein-based multimers, called engineered multimers, thereby expanding said multimer towards further modifications required for certain applications.

A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions. Protein secondary structure elements (SSEs) typically spontaneously form as an intermediate before the protein folds into its three dimensional tertiary structure. The two most common secondary structural elements of proteins are alpha helices and beta (β) sheets, though β-turns and omega loops occur as well. Beta sheets consist of beta strands (also β-strand) connected laterally by at least two or three back-bone hydrogen bonds, forming a generally twisted, pleated sheet. A β-strand is a stretch of poly-peptide chain typically 3 to 10 amino acids long with backbone in an extended conformation. A β-turn is a type of non-regular secondary structure in proteins that causes a change in direction of the polypeptide chain. Beta turns (β turns, β-turns, β-bends, tight turns, reverse turns) are very common motifs in proteins and polypeptides, which mainly serve to connect β-strands.

By “recombinant polypeptide” is meant a polypeptide made using recombinant techniques, i.e., through the expression of a recombinant or synthetic polynucleotide, which may be obtained in vitro and/or in a cellular context. When the chimeric polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation. By “isolated” or “purified” is meant material that is substantially or essentially free from components that normally accompany it in its native state.

“Homologue”, “Homologues” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. The percentage of amino acid identity as provided herein is preferably in view of a window of comparison corresponding to the total length of the native or natural wild-type protein, or of the specific amino acid sequence referred to.

The term “wild-type” refers to a gene or gene product isolated from a naturally occurring source, or included in a cell, cell line or organism. A wild-type gene or gene product is that which is most frequently observed in a population and is thus arbitrarily designed the “normal” or “wild-type” form of the gene or gene product a observed in nature. In contrast, the term “modified”, “engineered”, “mutant” or “variant” refers to a gene or gene product that displays modifications in sequence, post-translational modifications and/or functional properties (i.e., altered characteristics) when compared to the wild-type or naturally-occurring gene or gene product. A knock-out refers to a modified or mutant or deleted gene as to provide for non-functional gene product and/or function. It is noted that naturally occurring mutants or variants may be isolated; these are identified by the fact that they have altered characteristics when compared to the wild-type gene or gene product, and a different sequence as compared to the reference gene or protein.

### DETAILED DESCRIPTION

The present invention relates to novel protein assemblies applicable in several constellations as next-generation biomaterials. The generation of the multimeric assemblies as disclosed herein is based on the unravelling of the structural and genetical basis of Bacillus endospore appendages (Enas), which led to a number of opportunities for engineering and modulating these protein assemblies for the production of rigid but flexible structures with specific properties and with potential in numerous applications. The identification of the Ena protein family as building blocks of these multimeric and fibrous assemblies, directly correlated self-assembling property of the proteins to the presence of a DUF3992 protein domain present in a panel of bacterial proteins, allowing to form multimeric assemblies. Furthermore, the presence of the DUF3992 domain, as determined by adherence to the DUF3992 HMM profile (as provided in Table 1) in combination with a conserved N-terminal connector region, comprising at least two conserved cysteine residues, as provided by the motif ZXₙC(C)XₘC, wherein Z is Ile, Phe, Leu or Val, n is 1 or 2 residues, m is 10-12 residues, C is Cys, and X is any amino acid, which allows to covalently connect the multimeric assemblies longitudinally into a rigid fiber. Flexibility of the fibers is retained though by the characteristic of a 12-15 aa spacer region near the N-terminus, allowing to maintain the gap between stacked multimers (see FIG. 3).

A Novel Prokaryotic Self-Assembling Protein Family, the Ena Proteins.

A first aspect of the present invention relates to a self-assembling protein subunit, which comprises a DUF3992 domain, providing for the structural element required to obtain a self-assembling protein multimeric assembly under permissive buffer conditions. In this context, ‘self-assembly’ refers to the spontaneous organization of molecules in ordered supramolecular structures thanks to their mutual non-covalent interactions without external control or template. The chemical and conformational structures of individual molecules carry the instructions of how these are assembled. The same or different molecules may constitute the building blocks of a molecular self-assembling system. Generally, interactions are established in a less ordered state, such as a solution, random coil, or disordered aggregate leading to an ordered final state, which can be a crystal or folded macromolecule, or a further assembly of macromolecules. The association of small molecules or proteins into well-ordered structures is driven by thermodynamic principles, thus, based on energy minimization. The interactions involved in the molecular assembly process are electrostatic, hydrophobic, hydrogen bonding, van der Waals interactions, aromatic stacking, and/or metal coordination. Although non-covalent and individually weak, these forces can generate highly stable assemblies and govern the shape and function of the final assembly (Lombardi et al., 2019). Said self-assembling protein subunits described herein, and called Ena proteins herein, are capable of forming self-assembling multimers and protein fibers envisaged herein to be applied in different settings and biomaterials. The multimeric or fibrous assemblies can be obtained from the pre-existing components termed building blocks, or subunits, more specifically the isolated self-assembling proteins as described herein, the Ena proteins.

Moreover, other embodiment described herein relate to ‘modified’ or ‘engineered’ building blocks or protein subunits, or assemblies, as referred to herein, and are defined as being designed or derived from the existing (native) ones obtained by changing the chemical composition, the length, and the directionality of interactions to create new units, or units with a new functionality, which contain all the necessary information that encodes their self-assembly. By controlling environmental variables, the system reaches a new thermodynamic minimum leading to a different ordered structure. In most cases, because the protein subunit self-assembly occurs by non-covalent interactions, their self-assembly is reversible and sensitive to the environment and the activity can be tuned controlling the association and the dissociation of the proteins. The self-assembling property of these proteins is provided by the presence of the DUF3992 domain.

‘Domain of Unknown Function’ or ‘DUF’ protein families are designated as such as a tentative name and tend to be renamed to a more specific name (or merged to an existing domain) after a protein function is identified. So the present invention in fact defines for the first time a function of self-assembly to the prokaryotic DUF3992 domain-containing proteins that further also match the Ena1B protein fold, as described herein, even though, the DUF3992-containing proteins are in the PFAM database known as a family of proteins that is functionally uncharacterised, and found in bacteria, typically between 98 and 122 amino acids in length. The PFAM database (version 33.1) also mentions that there is a single completely conserved residue T that may be functionally important (El-Gebali et al. 2019, The Pfam database; http://pfam.xfam-org/family/PF13157). This ‘Domain of Unknown Function’ 3992 is structurally characterized by the Hidden Markov models (HMM) obtained according to alignment of the 64 bacterial proteins known (Pfam-B_480 release 24.0) to comprise this particular DUF3992 protein domain, as also provided in the PFAM database for the PFAM13157 family (also see Table 1 as provided herein). The HMM profile for DUF3992 domain proteins of PFAM13157 family is also shown on http://pfam.xfam.org/family/PF13157#tabview=tab4 and should be interpreted as in Wheeler et al. (2014): ‘hidden Markov models are shown by drawing a stack of letters for each position, where the height of the stack corresponds to the conservation at that position, and the height of each letter within a stack depends on the frequency of that letter at that position.’

This group of spontaneously assembling proteins comprising the DUF3992 domain, previously indicated in the databases as hypothetical proteins of unknown function may hence now be part of the annotation constituting the definition of the bacterial Ena protein family. So, the Ena protein family is defined as bacterial DUF3992 classifying proteins based on their HMM profile aligning with the one presented herein in Table 1, with a length of about 100 to 160 amino acids, with the capacity to spontaneously assemble into higher structures such as multimers, and preferably said multimers preferably having the capacity to further assemble into fibrous structures, stabilized by the formation of longitudinal covalent disulphide bridges. Furthermore, the structural definition of the Ena proteins relates to these bacterial DUF3992 self-assembling proteins with an Ena fold, wherein aid Ena fold comprises: an 8-stranded β-sandwich, with sheets in BIDG and CHEF topology, as described herein, and as derivable from the matching of the (predicted) fold based on the amino acid sequence, as compared to the reference Ena1B cryoEM structure fold provided herein with a Z-score of 6.5 or more, and with an N-terminal ‘Ntc’ element containing a conserved Z-Xₙ-C(C)-Xₘ-C motif for covalent connection to preceding subunits in the fiber, wherein X=any amino acid, Z=Leu/Val/Ile/Phe, n=1 to 2 residues, m=10 to 12 residues, and C=Cys.

More specifically, the DUF3992 domain-containing protein subunits in the multimers as described herein are non-covalently linked to each other through β-sheet augmentation, a structural feature known in the art and previously described for instance in Remaut and Waksman (2006) as a staggering of protein subunits via electrostatic interactions between a β-strand from one of the proteins binding to the edge of a β sheet in the other protein (also see FIGS. 2D, 2E and 3C). Finally, the bacterial DUF3992 domain-containing self-assembling proteins are provided herein by SEQ ID NOs: 1-80 and 145-146, and may be simply verified to fall under this Ena protein family by applying the present definition, i.e. by verifying whether a newly discovered protein is a member of this protein family, through a simple HMMR analysis (as provided for instance https://www.ebi.ac.uk/Tools/hmmer/ and based on the matrix provided herein as Table 1) which allows the skilled person to define whether the protein comprises a DUF3992 domain, and compare its fold, which may be predicted simply based on the amino acid sequence, applying a structure matching tool, as known to the skilled person, and as exemplified herein, to assure the structure is provided as an Ena fold, i.e. having a matching fold with a Z score of at least 6.5 as compared to the Ena1B structure as provided in PDB7A02. Moreover, whether a protein with a DUF3992 domain has the propensity to self-assemble and appear as a multimer of at least seven, preferably six to twelve protein subunits, as claimed herein, may be determined by tests as known by the skilled person, for instance, but not limited to SDS-PAGE, dynamic light scattering analysis, size-exclusion chromatography, or preferably negative stain transmission electron microscopy.

The DUF3992 domain-containing self-assembling Ena proteins as disclosed herein are N-terminally characterized by conserved cysteine residues favouring the formation of rigid pili or appendage assemblies, as observed on Bacillus endospores. Based on this observation, the capacity of this self-assembling protein family to form fibers in vitro was investigated herein (see FIGS. 13-14). These structural features of these protein subunits identified herein allow to strongly connect covalently between several self-assembled multimers, via the presence of said cysteine residue side chains. So, the family of bacterial Ena proteins constitute a DUF3992 domain and at least one or more conserved cys residues in the N-terminal region. More specifically, said Ena protein family has been identified herein as containing Ena1, Ena2 and Ena3 proteins, wherein Ena1 and Ena2 were each shown to contain 3 members (A, B, C), all comprising specific amino acid residue consensus motifs in their N- and C-terminal regions, as described in detail further herein. Said Ena gene/protein family is also structurally and phylogenetically in more detail described in the Examples, revealing that an ‘Ena1’ or ‘Ena2’ gene cluster is present in Bacillus species, allowing S-type fiber formation, and in addition a single Ena3A gene, required for L-type fiber formation. The Bacillus S-type native protein fibers as described herein require all 3 members, Ena1/2 A, B and C to be formed on the endospores. Surprisingly, Ena1/2C was not structurally present in the ex vivo fiber constellation, so the Ena1/2C protein, although having self-assembling properties, has a different contribution to the fiber formation during sporulation in vivo. Strikingly, recombinant expression of either of these 3 members, Ena1/2A, B, or C, resulted in the formation of multimers in a host cell. Moreover, recombinant expression of a single Ena1/2 A or B protein without steric block (e.g. the wild type sequence), even allowed formation of S-type like fibers within the host cell. Recombinantly expressed Ena1C results in a different type of multimeric assembly, and showed disc-type multimers. Furthermore, recombinantly expressed Ena1/2A or B, when arrested by a steric block, as defined further herein, forms helical turns or arc-type multimers. Finally, the Ena3A protein, encoded by an operon comprising a single Ena subunit in the Bacillus genome also comprises a DUF3992-domain, and has a conserved Cys residue patterns in its N-terminus. The C-terminal region is more diversified from the Ena1/2 proteins though. This Ena3A has been identified to constitute the L-type fibers observed on Bacillus endospores. The L-type fibers appear as disc-like multimers which are longitudinally stacked via disulphide bonds for stabilizing the fiber.

Said Ena protein is defined herein as the proteins of PFAM 13157, constituted of bacterial DUF3992 domain-containing proteins, as characterized by its specific HMM profile, and as described in the Examples provided herein, further demonstrating to have a conserved Cys residue profile (see FIGS. 16-19), preferably as defied herein for S-type and L-type fiber forming subunits, and more preferably also the conserved C-terminal motif as described herein, and specifically comprising the members of the bacterial Ena1, Ena2, and Ena3 protein subfamilies. The Ena protein family has its origin in the bacterial Bacillus spp. group and is limited to protein sequences originating from bacteria. Structurally, Ena proteins are characterized by a jellyroll 3D structure composed of two juxtaposed β-sheets, wherein said β-sheets provide for a topology consisting of strands BIDG and CHEF, and further comprising a flexible N-terminal region consisting of an ‘extension’ or ‘connector’, typically the first 10-20 residues in length, followed by a spacer, to ensure the physical distance between multimers in the stacked fiber, about 5-16 residues in length (see FIGS. 8, and 17-19). So, in a particular embodiment, the multimer of the invention comprises at least 6, preferably 6 to 12, Ena protein subunits, wherein the BIDG β-sheet of subunit (i) is augmented with CHEF β-sheet of (i−1) and CHEF β-sheet of subunit (i) is augmented with BIDG β-sheet of (i+1). More particular, the multimer may comprise 7 to 12, 7 to 11, 7 to 10, 8 to 10, or 9 protein subunits, or exactly 7, 9, 10, 11 or 12 subunits.

In view of the phylogenetic and functional characterization of this family, an ‘Ena protein’, as used herein, is exemplified, but not limited to the list of Bacillus proteins depicted in SEQ ID NO:1-80, SEQ ID NO:145 or SEQ ID NO:146, disclosing representative proteins for each cluster of each Ena protein family member, exemplified further herein by Bacillus cereus NVH 0075-95 383 Ena1A (SEQ ID NO:1), Ena1B (SEQ ID NO:8), and Ena1C (SEQ ID NO:15) and Bacillus cytotoxicus NVH 391-98 Ena2A (SEQ ID NO: 21), Ena2B (SEQ ID NO: 29), Ena2C (SEQ ID NO: 38), and Bacillus cereus Ena3A (SEQ ID NO:49), and a number of homologues and/or orthologues in other bacterial strains, wherein each orthologous sequence of a family member has at least 80% identity to the sequence used herein as defined over their total length (also see Examples ‘Phylogenetic analysis’; and FIGS. 16-19). More specifically, Bacillus cereus NVH 0075-95 383 Ena1A and Ena1B proteins are depicted in SEQ ID NO:1 and SEQ ID NO:8, respectively, and any bacterial homologue thereof with at least 80% amino acid identity over the full sequence as comparison window, comprising the DUF3992 domain and N- and C-terminal conserved Cys residues is a candidate orthologue (FIGS. 16-17). Bacillus cereus NVH 0075-95 383 Ena1C protein is depicted in SEQ ID NO: 15 and any bacterial homologue thereof with at least 60, 70 or 80% amino acid identity over the full sequence as comparison window, comprising the DUF3992 domain and N- and C-terminal conserved Cys residues is a candidate orthologue (FIG. 18). Similarly, Bacillus cytotoxicus NVH 391-98 Ena2A and Ena2B proteins are depicted in SEQ ID NO:21 and SEQ ID NO:29, respectively, and any bacterial homologue thereof with at least 80% amino acid identity over the full sequence as comparison window, comprising the DUF3992 domain and N- and C-terminal conserved Cys residues is a candidate orthologue (FIG. 16-17). Bacillus cytotoxicus NVH 391-98 Ena2C protein is depicted in SEQ ID NO: 38 and any bacterial homologue thereof with at least 60, 70 or 80% amino acid identity over the full sequence as comparison window, comprising the DUF3992 domain and N- and C-terminal conserved Cys residues is a candidate orthologue (FIG. 18). Bacillus cereus Ena3A protein is depicted in SEQ ID NO: 49 (multispecies ref.) and any bacterial homologue thereof with at least 60, 70 or 80% amino acid identity over the full sequence as comparison window, comprising the DUF3992 domain and N- and C-terminal conserved Cys residues is a candidate orthologue (FIG. 19).

Multimer Assemblies.

A second aspect of the invention relates to a protein multimeric assembly, or multimer, which comprises at least 7, preferably between 7 and 12, or more self-assembling protein subunits with a ‘Domain-of-Unknown-Function 3992’ (DUF3992) domain protein and typical N-terminal conserved region, wherein said protein subunits are non-covalently connected to each other.

Said self-assembling DUF3992 domain-containing protein subunits more specifically relate to proteins subunits comprising an Ena protein sequence, and/or an engineered Ena protein sequence.

Another embodiment discloses the multimer comprising 7-12 protein subunits wherein said protein subunits comprise Ena proteins, and/or an engineered Ena protein form thereof. In specific embodiments said multimers comprise proteins subunits selected from Ena proteins as depicted in SEQ ID NOs:1-80, 145-146, or a homologue with at least 60% identity of any one thereof, or at least 70%, or at least 80%, or at least 85%, or at least 90%, or at least 95%, or at least 97% of any one thereof, a functional orthologue thereof, and/or an engineered Ena protein form thereof. These multimers as described herein are formed by self-assembly of protein subunits comprising a DUF3992 domain and defined to consist of 6, 7, 8, 9, 10, 11 or 12 protein subunits (FIG. 14-15). These protein multimers are defined herein to function for a number of applications in the format of the multimer ‘as such’, meaning that the multimers are defined to be independent units within a solution, a cell, or another type of in vitro environment, while such multimers of DUF3992 domain or Ena protein subunits in itself are not found in nature, and do not form or assemble ‘as such’ in vivo or in natural conditions due to their propensity to form fibers. S-type fibers are not composed of separate multimers, but comprise multimeric Ena structures that continue into a longitudinal fiber as a continuous helical structure formed by lateral non-covalent interactions, specifically β-sheet augmentation, between subsequent protein subunits. In addition, due to the presence of conserved Cys residues in the N-terminal and C-terminal region these are further rigidified by covalent disulphide bridges. To form Ena1/2A or Ena1/2B multimers ‘as such’ as a stand-alone product, a ‘steric block’ is required to prevent further assembling of the multimers (See FIGS. 13A and 14A). Said specifically defined multimers are thus arrested in their fiber growth, for instance by sterically hindering the N-terminus from going in covalent connections with other multimers. A ‘sterically frustrated’ or ‘sterically hindered’ or ‘sterically blocked’, as interchangeably used herein, N-terminal region is defined herein as a structural difference to the naturally occurring Ena protein N-terminus wherein said structural difference results in steric hindering of the N-terminus from covalent linkage with other proteins or multimers. For instance, by addition of a heterologous N-terminal tag of at least 1-5, 6, 7, 8, 9, 10, 11, 12, 13, 14, 15 or more amino acid residues to one or more wild type Ena protein subunits, an ‘engineered or modified’ heterologously tagged Ena protein is formed which will arrest outgrowth of the multimer into longitudinal direction, as for instance by preventing covalent linkage of different multimers. Alternatives to sterically frustrate the N-terminus of the protein subunit of said multimers are for instance a C-terminal extension or tag, required for longitudinal interaction, especially for S-type fiber formation. Or an alternative could be to add a chemical linker which sterically blocks any disulphide linking of the N- or C-terminal connectors, or by mutating the N-terminal Ena protein sequence to remove cysteines, or creation of an Ena protein variant to sterically hinder disulphide bridge formation with other multimers. A particular embodiment thus relates to a multimer as described herein, wherein at least one protein subunit further comprises a heterologous N- and/or C-terminal tag or extension or connector of at least 1-5, 6, 7, 8, 9, 10, 11, 12, 13, 14, 15 or more amino acids to form a steric block. So to obtain a decamer, undecamer or dodecamer of Ena1/2A and/or Ena1/2B assemblies ‘as such’, the presence of a steric block at the N-terminus is desired (see FIG. 14-15) to prevent further assembly of these multimers into fibers. These multimers as stand-alone protein units may thus be formed upon engineering of at least one protein subunit of said multimer, as described also in more detail further herein. A particular embodiment thus relates to the multimer as described herein, which is an arrested multimer set forth as a single turn or helical arc multimer, with an N- and/or C-terminal region or connector that is sterically frustrated.

Alternatively, the Ena1/2C protein has been shown to form ring-like or disc-like multimers when recombinantly expressed. A closed circular multimer or disc-like structure is formed in vitro, with or without a sterically frustrated N- and/or C-terminal region. Even more, in particular cases even a recombinantly expressed truncated Ena1/2C protein, lacking the first N-terminal connector region, is capable of self-assembly and to assemble into multimers. In one embodiment, these Ena1C constituting multimers may consists of a heptamer or a nonamer, with 7 or 9 subunits, respectively (see also FIGS. 14B and 15B).

The recombinantly produced Ena1C multimer or nonameric ring-structure may be further engineered by adding a heterologous N- or C-terminal tag, by mutation or insertions to adapt the Ena1C multimeric assemblies as biofunctional and structural tools.

In a specific embodiment, said multimer as described herein, comprising six to twelve protein subunits comprising a DUF3992 domain-containing protein, or specifically an Ena protein, a homologue thereof or an engineered form thereof, is an isolated multimer. Said isolated multimer is obtained by recombinant expression of a chimeric gene as described herein, to produce the multimer ‘as such’, optionally followed by purification of said multimers from the production host. One embodiment thus relates to said isolated multimer consisting of at least 6, or preferable 7-12 subunits, or an engineered multimer or a multimer comprising at least one engineered protein subunit as compared to the protein subunit its natural counterpart or wild type protein form. In specific embodiments, the protein subunits of the multimers as described herein may be homomeric multimers, or heteromeric multimers, the latter may comprise identical DUF3992 subunits, or consist of wild type Ena protein subunits and engineered Ena protein subunits, such as for instance tagged Ena proteins, or mutant Ena protein subunits. The heteromeric multimers may consist of one type of Ena protein or several types of Ena protein members.

Overall, the those multimers as defined herein to comprise at least seven DUF3992 domain-containing protein subunits, which may be at least one Ena protein as defined herein, and wherein said protein subunits are non-covalently linked via β-sheet augmentation, may comprise at least one engineered Ena protein subunit, which is defined herein as a non-naturally occurring Ena protein subunit, with the aim to prevent further oligomerisation and covalent interaction triggered by the N-terminal and/or C-terminal regions forming inter-multimeric disulphide bridges, and/or to acquire additional functionalities or properties for said multimeric assemblies.

An ‘engineered DUF3992-containing protein subunit’ as defined herein, or an ‘engineered Ena protein’ as defined herein, relates to non-naturally occurring forms of DUF3992-containing or Ena proteins, respectively, which is still capable of self-assembling and forming multimeric or fibrous structures. Engineered or modified or modulated proteins subunits or protein subunit variants, as interchangeably used herein, may show differences on their primary structural feature level, i.e. on their amino acid sequence as compared to the wild type (Ena) protein, as well as by other modifications, i.e. by chemical linkers or tags. An engineered protein subunit may thus concern a mutant protein, comprising for instance one or more amino acid substitutions, insertions or deletions, or a fusion protein, which may be a tagged or labelled protein, or a protein with an insertion within its sequence or its topology, or a protein formed by assembly of partial or split-Ena proteins, among other modifications. So in one embodiment, an engineered Ena protein is disclosed, wherein said engineered Ena protein is a modified Ena protein as compared to native Ena proteins, and is a non-naturally occurring protein. Non-limiting examples as provided herein relate to N- or C-terminally tagged Ena proteins, more specifically with a heterologous tag of at least 1, 2, 3, 4, 5, 6, 7, 8, 9, 10, 11, 12, 13, 14, 15 or more amino acid residues long, to acquire sterically frustrated Ena protein subunits for multimer formation without forming any fibrous assemblies; Ena mutant or variant proteins; Ena protein fusions or Ena proteins with a heterologous peptide or protein inserted within one of its exposed loops between β-strands, or Ena proteins formed upon assembly of Ena split-protein parts separately expressed in a host.

A tag is a ‘heterologous tag’ or ‘heterologous label’ resulting in a ‘heterologous fusion’ if it is not naturally occurring in the wild-type protein sequence, and is added for application purposes, such as for facilitating purification of the protein, or for assembling multimers sterically hindered in outgrowth of fiber formation. The term “detectable label”, “labelling”, or “tag”, as used herein, refers to detectable labels or tags allowing the detection, visualization, and/or isolation, purification and/or immobilization of the isolated or purified (poly-)peptides described herein, and is meant to include any labels/tags known in the art for these purposes. Particularly preferred are affinity tags, such as chitin binding protein (CBP), maltose binding protein (MBP), glutathione-S-transferase (GST), poly(His) (e.g., 6×His or His6), Strep-tag®, Strep-tag II® and Twin-Strep-tag®; solubilization tags, such as thioredoxin (TRX), poly(NANP) and SUMO; chromatography tags, such as a FLAG-tag; epitope tags, such as V5-tag, EPEA-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase; and (other) enzymatic labels (e.g., peroxidase, alkaline phosphatase, beta-galactosidase, urease or glucose oxidase). Also included are combinations of any of the foregoing labels or tags.

Said functional engineered protein subunits or engineered Ena protein subunits or monomers, preferably engineered by addition of a tag, may further be capable of forming an arrested multimer, or an arrested fiber, in itself, as a homomultimeric assembly of engineered Ena protein subunits, or as a heteromultimeric assembly combining engineered and non-engineered (e.g. wild type) Ena protein subunits.

In a particular embodiment, the proteins subunit may be engineered Ena proteins comprising at least one Ena mutant or Ena variant protein subunit. For example, though not-limiting, such Ena mutants or variants can be derived from the structural information demonstrating where modification or mutation of surface sidechains of the multimer or protein subunit is feasible (see also FIG. 15). Substitutions that are possible to in analogy with those proposed for Ena1B subunit mutants are shown in FIG. 15 for the Ena1B as depicted in SEQ ID NO:8, for residue A31, T32, A33, T57, T61, V63, V69, T70, T72, A73, T76, V78, T96, L98, T100, and A101. Examples of relevant replacement residues comprise Cysteine or Lysine, or non-natural amino acids amenable to click chemistry, such as those with an azide side chain.

Furthermore, an example of insertion sites in Ena1B (SEQ ID NO:8) is depicted in FIG. 15 by the positions located the loop connecting the following β-strands: B-C strands with residues A30 to A33; D-E strands with residues T55 to P59; E-F strands with residues S66 to T72; and the H to I strands with the loop of residue G99 to A103. An insertion of a heterologous protein or peptide or linker in such a loop may consist of an amino acid sequence up to 400 residues long, and still retain the folding and structural features required for multimer formation. Specifically how to create such an insertion variant or functional mutant engineered Ena protein may be envisaged as for example by modifying the primary amino acid sequence of for instance Ena1B as such: reordering the sequence by first inserting a single residue peptide or a (poly)peptide between β strands E and F by cleaving the Ena1B protein at residue S66, and adding the insert its N-terminal residue to the C-term of S66, and the insert its C-terminus to the N-term of G67 of Ena1B. An insertion may also be created by removing a number of amino acids from the loop of said Ena protein, for example the Ena1B sequence residues S66 to T72 may be replaced with an insert. The skilled person is aware of how to create similar inserts in different Ena protein loop areas as provided herein based on the disclosed structural features of the Ena proteins, and may also thereby create similar insertions for Ena homologues or engineered Ena protein forms thereof.

The N-terminal region and C-terminal region as defined herein for Ena proteins refers to the wild type Ena protein sequence. For said wild type (or substitution/mutant variant) Ena proteins, the ‘N-terminal region’ is defined as the first part of the Ena protein sequence comprising a flexible N-terminal connector followed by a spacer, and the first β-strand B of the typical BIDG CHEF β-sheets composing the jellyroll folding of said Ena protein subunit. The ‘C-terminal region’ of the Ena proteins as defined herein is the end of the protein sequence comprising the last β-strand I of the BIDG CHEF β-sheets and possible residual C-terminal residues thereafter.

One application one may consider is to modify the Ena protein subunit in an engineered Ena protein format whereby another functional moiety or protein, such as for instance an antibody or alike, is fused to said Ena protein or Ena multimer, providing for a functionalized multimer, optionally coupled to a surface or support.

In order to make structurally attractive fusions, the skilled person may consider engineering the Ena protein as a circularly permutated protein. The term “circular permutation of a protein” or “circularly permutated protein” refers to a protein which has a changed order of amino acids in its amino acid sequence, as compared to the wild type protein sequence, with as a result a protein structure with different connectivity, but overall similar three-dimensional (3D) shape. A circular permutation of a protein is analogous to the mathematical notion of a cyclic permutation, in the sense that the sequence of the first portion of the wild type protein (adjacent to the N-terminus) is related to the sequence of the second portion of the resulting circularly permutated protein (near its C-terminus), as described for instance in Bliven and Prlic (2012). A circular permutation of a protein as compared to its wild protein is obtained through genetic or artificial engineering of the protein sequence, whereby the N- and C-terminus of the wild type protein (as defined above herein for Ena proteins) are ‘connected’, and the protein sequence is interrupted or cleaved at another site, to create a novel N- and C-terminus of said protein. The circularly permutated Ena protein of the invention is thus the result of a connected N- and C-terminus of the wild type Ena protein sequence, and a cleavage or interrupted sequence at an accessible or exposed site (preferentially a β-turn or loop) of said Ena protein subunit, whereby the folding is retained or similar as compared to the folding of the wild type Ena protein. Said connection of the N- and C-terminus in said circularly permutated scaffold protein may be the result of a peptide bond linkage, or of introducing a peptide linker, or of a deletion of a peptide stretch near the original N- and C-terminus if the wild type protein, followed by a peptide bond or the remaining amino acids. This rearrangement of the N- and C-terminus of the resulting Ena protein is referred to as the secondary N- and C-terminus.

Finally, the multimers as described herein provide for numerous applications in the field of next-generation biomaterials. In one embodiment, said multimers may be coupled to a solid surface, and as such provide for modified surfaces with properties of having an extreme resilient behaviour, thus being very stable and rigid materials.

Fibrous Assemblies.

Another aspect of the invention relates to recombinantly produced fibers comprising at least two multimers, wherein said multimers comprise at least 7 protein subunits, or 7-12 subunits, which comprise a self-assembling DUF3992 domain-containing protein, in particular an Ena protein, wherein said protein subunits are non-covalently connected via β-sheet augmentation, and wherein said multimers are longitudinally stacked and covalently connected via at least one disulphide bridge. The protein fibers may thus be produced in a non-natural host, recombinantly, in cellulo and/or in vitro, and may comprise heteromeric or homomeric multimers. When heteromeric protein fibers are envisaged, the multimers may comprise one or more self-assembling DUF3992-domain-containing Ena proteins, or alternatively the protein subunits are identical except for that one or more subunit is an engineered protein form thereof. Homomultimeric protein fibers may be generated by recombinantly expressing a specific Ena protein or Ena protein mutant, variant or engineered Ena protein in a host cell. Any recombinantly produced protein fiber comprising one or more Ena protein subunits will be a non-naturally occurring fiber since the ruffles observed on the in vivo Bacillus fibers (see Examples) have never been seen in the recombinantly produced fibers.

In a specific embodiment, the protein subunits or multimers as described herein comprise an ‘N-terminal region’ or ‘N-terminal connector’ or ‘N-terminal connector region’, as used interchangeably herein, with a conserved amino acid residue sequence motif depicted as ZXₙCCXₘC, wherein Z is Leu, Ile, Val or Phe, and X is any amino acid, n is 1 or 2 residues, and m is 10-12, and comprising a ‘C-terminal region’ or ‘C-terminal receiver region’, as used interchangeably herein, with a conserved amino acid motif depicted as GX_{2/3}CX₄Y, wherein X is any amino acid, to allow S-type fiber formation of said multimers by longitudinally connecting the Cys present in said motifs to form covalent disulphide bonds. In a specific embodiment, said protein fiber formed by these multimers has a helical structure (e.g. FIGS. 13a-14a). The protein fibers may only be formed when the multimers are thus not sterically hindered.

In another embodiment, an ‘engineered multimer’ for modulating the rigidity and/or elasticity of said protein fiber is produced wherein the N-terminal region of one or more protein subunits comprises a N-terminal conserved motif ZXₙCCXₘC, wherein Z is Leu, Ile, Val or Phe, and X is any amino acid, n is 1 or 2 residues, but with m being 7, 8 or 9 amino acid residues instead of 10-12 residues, resulting in a shorter N-terminal region (as compared to Ena1A of SEQ ID NO:1 or Ena1B of SEQ ID NO:8, for instance), or with m being between 13 and 16 residues, resulting in a longer N-terminal region terminal region (as compared to Ena1A of SEQ ID NO:1 or Ena1B of SEQ ID NO:8 for instance). Said engineered multimers may still allow to form covalent S-S bridges via said cysteines with the C-terminal receiver motif GX_{2/3}CX₄Y in the assembly of an S-type or helical fiber, but may be of lower stability or rigidity as compared to the ones where m is 10-12 residues. The formation of S-type or helical fibers may be possible without disulphide bridge formation, though this will result in much less stable and lower resilient fiber structures. Indeed, as supported herein, the fiber structures that comprise the N-terminal cysteine covalent linking provide for a stability that allows for instance the endospore appendages to survive in harsh conditions. The disulphide bonds present in the lumen of the fibers allow for this strength and are therefore preferred in the fibers.

Furthermore, L-type protein fibers comprising disc-type multimers are also longitudinally cross-linked via covalent linkage between N-terminal conserved Cys residues and multimers of the preceding layer connector. Said fibers may be formed by recombinant expression of Ena3, as depicted in SEQ ID NOs:49-80 or a homologue with at least 80% of any one thereof. Said Ena3 proteins being functional in L-type fiber formation are further defined herein to contain an N-terminal connector with a conserved motif that is slightly adapted to the Ena1/2 A&B S-type fiber forming subunits, i.e. the motif wherein the second Cys may be replaced by another amino acid in some Ena3 proteins, so as defined by ZXₙC(C)XₘC, wherein Z is Leu, Ile, Val or Phe, and X is any amino acid, n is 1 or 2 residues, and m is 10-12, and comprising a ‘C-terminal region’ or ‘C-terminal receiver region’, as used interchangeably herein, with a conserved amino acid motif depicted as S-Z-N-Y-X-B, wherein Z is Leu or Ile, B is Phe or Tyr, and X is any amino acid, to allow L-type fiber formation of said multimers by longitudinally connecting the Cys present in said motifs to form covalent disulphide bonds. In a specific embodiment, said protein fiber formed by these multimers has a disc-like structure (e.g. FIGS. 13b-14b). The protein fibers may only be formed when the multimers are thus not sterically hindered.

For instance, by addition of a heterologous N-terminal tag of at least 1 to 5, 6, 7, 8, 9, 10, 11, 12, 13, 14, 15, or more amino acids, steric hinder will prevent or negatively affect disulphide bridge formation thereby preventing fiber formation, or resulting in partially formed fibers or less strong and less resilient or rigid fibers (see examples).

In a specific embodiment, the produced protein fiber comprising said at least 2 multimers are covalently linked through at least one disulphide bond between a side chain of a Cys residue of the N-terminal connector region of at least one protein subunit of one multimer with a Cys residue of a protein subunit of the receiver region of the multimer of the preceding layer in this longitudinal direction. In a preferred embodiment, there are at least two disulphide bonds formed between different multimers of the fiber, and most preferably each disulphide bond contains a sulphur atom from the cysteines in the N-terminal region of one or more protein subunits to make a bond to the sulphur atom of the cys present in the protein subunit of the preceding multimer of the fiber. In a specific embodiment said N-terminal region has two consecutive Cys in said conserved amino acid motif to both take part in a disulphide bridge with another multimer of the fiber. Other embodiments relate to said protein fibers as nanofibers comprising at least 2 multimers, wherein said multimers are stacked and covalently linked through disulphide bridge(s) formed by the first and second Cys residues of the N terminal conserved motif of protein subunit (i) and the Cys residue of the β-strand I of subunit (i−9) and B of subunit (i−10), respectively.

The protein fiber as described herein is thus composed from two or more multimers each comprising at least 7 protein subunits comprising a self-assembling DUF3992 domain-containing protein, as described herein, or more particular comprising an Ena protein or engineered Ena protein, wherein said protein subunits are non-covalently linked, and wherein said multimers are longitudinally stacked solely by forming covalent disulphide bonds between said stacked multimers. In said protein fibers, said multimers may be identical or different in composition. And said multimers may be engineered multimers for modulating the rigidity of the fiber, as defined herein. Furthermore, said at least two multimers of said protein fiber may be multimers comprising identical protein subunits, or comprising different protein subunits. Contrary to the L-type fibers, which comprise distinguishable multimeric discs that are only covalently connected via the disulphide bridges, the multimers present in S-type fibers will not be distinguishable as single units that are solely covalently connected, but will be a continuous β-sheet augmentation of protein subunits in a β-propeller helical structure, and additionally crosslinked every helical turn by disulphide bridges. So ‘a protein fiber comprising the multimers’ as used herein may refer to a protein fiber which is consisting of distinguishable separate disc-like multimers (e.g. comprising solely Ena3A-based protein subunits) solely connected via S-S bridges, or to a protein fiber compiled from helical-turn-like multimers (e.g. Ena1/2A and/or Ena1/2B protein-based), which are continuously non-covalently connected into a fibrous helical structure, and further crosslinked via S-S bridges.

Furthermore, alternative embodiments comprise an engineered protein fiber, which is defined as a fiber comprising two or more multimers, as described herein, wherein at least one multimer is an engineered multimer, as defined herein, and/or wherein at least one protein subunit is an engineered protein subunit, as defined herein.

Another embodiment relates to a recombinantly produced or in vitro produced and purified protein fiber, wherein said fiber may be obtainable by recombinant or in vitro expression of the chimeric gene as described further herein. Said in vitro produced fiber may be an S-type fiber as disclosed herein, and may be formed by multimers comprising Ena1A and/or Ena1B protein, and/or an engineered form thereof. Said in vitro produced fibers are not occurring in nature, such as on Bacillus endospores, for which is it clear that Ena1A, Ena1B and Ena1C are indispensably required to form S-type fibers in vivo (see Examples). A specific embodiment relates to said in vitro produced protein fiber which is an engineered protein fiber in that the multimers of said proteins fiber comprise at least one engineered multimer, as described herein, or at least one multimer comprising an engineered protein subunit, as described herein, in particular at least one engineered Ena protein, as described herein. A further embodiment provides for an engineered protein fiber, wherein the protein fiber as described herein is fused to another protein or is conjugated to another moiety, such as a chemical moiety, or a functional moiety.

Another aspect of the invention provides for a chimeric gene or chimeric construct, which comprises DNA elements comprising at least a heterologous promoter or regulatory element operably linked to a nucleic acid sequence which upon expression controlled by said promoter or regulatory element results in a nucleic acid molecule encoding a protein subunit or protomer containing a self-assembling protein, as defined herein, and wherein said heterologous promoter or heterologous regulatory element sequence is originating from another source as (or is different to the native form of) the nucleic acid sequence encoding the bacterially derived self-assembling protein. In a further embodiment said chimeric gene comprises a heterologous promoter element or regulatory expression element operably linked to a nucleic acid molecule encoding an Ena protein, as described herein, or an engineered Ena protein thereof, which may be an Ena mutant or variant protein, an extended Ena protein (sterically frustrated to prevent fiber formation) or a fusion protein. Moreover, said chimeric construct may be present in an expression cassette, or as part of a cloning or expression vector for production of the protein in vitro.

An “expression cassette” comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”.

The term “vector”, “vector construct,” “expression vector,” or “gene transfer vector,” as used herein, is intended to refer to a nucleic acid molecule capable of transporting another nucleic acid molecule to which it has been linked, and includes any vector known to the skilled person, including any suitable type. including, but not limited to, plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Expression vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Suitable vectors have regulatory sequences, such as promoters, enhancers, terminator sequences, and the like as desired and according to a particular host organism (e.g. bacterial cell, yeast cell). Cloning vectors are generally used to engineer and amplify a certain desired DNA fragment and may lack functional sequences needed for expression of the desired DNA fragments. The construction of expression vectors for use in transfecting prokaryotic cells is also well known in the art, and thus can be accomplished via standard techniques (see, for example, Sambrook, et al. Molecular Cloning: A Laboratory Manual, 4th ed., Cold Spring Harbor Press, Plainsview, N.Y. (2012); and Ausubel et al., Current Protocols in Molecular Biology (Supplement 114), John Wiley & Sons, New York (2016), for definitions and terms of the art.

A further embodiment relates to a host cell expressing the chimeric gene as described herein, thereby possibly resulting in a host cell comprising the protomers or protein subunits of the multimers or forming the fibers as described herein. ‘Host cells’ can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Sambrook et al., Molecular Cloning: A Laboratory Manual, 4th ed., Cold Spring Harbor Press, Plainsview, N.Y. (2012); and Ausubel et al., Current Protocols in Molecular Biology (Supplement 114), John Wiley & Sons, New York (2016). Recombinant host cells, in the present context, are those which have been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction. A DNA construct capable of enabling the expression of the chimeric protein of the invention can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (2012), Wu (ed.) (1993) and Ausubel et al. (2016). Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Animal host cells suitable for use with the invention include insect cells and mammalian cells (most particularly derived from Chinese hamster (e.g. CHO), and human cell lines, such as HeLa. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively, the host cells may also be transgenic animals.

A specific embodiment relates to a Bacillus spp. cell comprising a chimeric gene encoding an Ena protein, or engineered Ena protein, as defined herein, so that upon sporulation of said Bacillus spp. the gene is expressed to form modified endospores, with (engineered) Ena protein for self-assembly into engineered Ena multimers and fibers in vivo. So a specific embodiment relates to a Bacillus spore or endospore comprising or displaying recombinant protein fibers comprising Ena protein or engineered Ena protein. Said engineered fibers on said spores may be advantageous for applying the spores in a certain environment or context.

Another embodiment relates to a method to produce such a modified endospore, comprising the steps of recombinant expression of a chimeric gene(s) as described herein in a spore-forming bacterial cell, and incubate in conditions for inducing sporulation.

Another aspect of the invention relates to a modified surface or solid support, which contains the (engineered) multimer or protein fiber of the invention. Particularly a modified surface is disclosed wherein a self-assembling Ena protein subunit as defined herein is covalently linked to a solid surface. A particular embodiment relates to said modified surface wherein at least one Ena protein subunit or engineered Ena protein is covalently linked to a solid support. Such a modified surface may be used as a nucleator surface allowing epitaxial growth to further form multimers and fibers as described herein, linked to said protein subunit and surface, when said modified surface comprising at least one Ena protein subunit is exposed to a solution comprising further Ena proteins, which will thus self-assemble with each other into multimers and upon covalent disulphide bridge formation form protein fibers outgrowing from said surface.

Surface immobilization may be envisaged as covalent binding of at least one (engineered) Ena protein subunit on said surface by using means known by the skilled person. Such means include, but are not limited to click chemistry, cross-linking to free amines (at the N-term, via Lysine) for example through NHS-chemistry, disulphide cross-linking, thiol-based cross-linking, addition of a tag (snap- or sortase tag for instance), fusion at N- or C-terminal end of the Ena protein to allow covalent attachment of the protein to a surface, as known in the art. The conditions in which a monomeric Ena subunit is coupled to the surface is envisaged to concern a denaturing buffer condition in a specific embodiment.

The protein fibers or engineered protein fibers may as well be fused or attached on the cell or microbial surface of the host, or can be nucleated onto a foreign surface that is exposed to a solution containing the Ena protein to obtain a modified surface comprising the fiber or engineered fiber.

Said surface immobilization may thus be accomplished herein on biological or synthetic surfaces. Biological surface includes the surface of a cell, of a bacterium, an (endo)spore, or other naturally occurring or recombinantly produced surfaces. High density surface expression of recombinant proteins is a prerequisite for successfully using cellular surface display in several areas of biotechnological applications in the fields of pharmaceutical, fine chemical, bioconversion, waste treatment and agrochemical production.

An artificial or synthetic surface may for instance include a bead, a slide, a chip, a plate, or a column. More particularly, the artificial surface may be particulate (e.g. beads or granules) or in sheet form (e.g. membranes or filters, glass or plastic slides, microtitre assay plates, dipstick, capillary devices) which can be flat, pleated, or hollow fibers or tubes. A range of biotechnological applications make use of the coating or activation of synthetic surfaces with protein assemblies, such as multimer compositions or fibers as described herein.

So the invention also provides for a system or in vitro method that couples the production of the Ena proteins or derivatives thereof with a self-assembling property that leads to the formation of multimeric and/or fibrous assemblies onto a synthetic surface and that displays these on said surface in a conformation for further specific capturing or displaying means and molecules to fulfil a certain goal in the biomedical or biotechnological field of biomaterials.

The invention further relates to directly applicable products obtained by generating the protein subunits, multimers or fibers or any engineered forms thereof in a particulate context. The self-assembling protein subunits according to the present invention indeed allow to self-assemble readily into multimeric assemblies as well as long, resilient, flexible nanofibers, which can be tailored for different functions through point mutations, peptide or protein fusions, and conjugates. Said engineered nanofibers with high rigidity and stability, even in harsh conditions, though with very high flexibility will provide for next-generation biomaterials. In one embodiment, such a biomaterial is present in the form of a thin protein film comprising the engineered protein fiber as described herein, and/or the protein fiber as described herein. As provided in the Example section (and e.g. FIGS. 8F and 12), with ‘thin’ it is meant that only a limited number of layers is possible as defined by the size of the fibers, similar to at least the diameter size of the Ena appendages observed on Bacillus, with several layers having a multiple of that diameter size (approx. 8 nm), so in the nanometer range. Such a thin film in fact provides for a dense and protected environment formed by the fibers. For example, increased resistance to detergents, chemicals, heat, UV and other harsh conditions as observed herein allow such a thin film to protect molecules on the opposite side of the film.

Another embodiment relates to a hydrogel comprising the engineered protein fiber of the invention, and optionally a protein fiber as described herein. In another embodiment, hydrogels are disclosed comprising an engineered multimer as described herein or a multimer comprising an engineered protein subunit as described herein. Hydrogels are known as water-swollen polymeric materials that maintain a distinct three-dimensional structure. They were the first biomaterials designed for use in the human body. Novel approaches in hydrogel design have revitalized this field of biomaterials research with applications in therapeutics, sensors, microfluidic systems, nanoreactors, and interactive surfaces. Hydrogels may self-assemble by hydrophobic, electrostatic or other types of molecular interactions. Designing hydrogel-forming polymers, using recognition motifs found in nature, enhances the potential for the formation of precisely defined three-dimensional structures.). The (engineered) multimers or protein fiber of the invention also provide for well-structured 3D building blocks to form a hydrogel, for which methods are known to the skilled person. The versatility of the revealed structures of the invention especially provide for an opportunity to manipulate its stability and specificity by modifying the primary structure, i.e. by using engineered proteins subunits, multimers or fibers of the invention for the successful design of a new class of hydrogel biomaterials. Furthermore, also hybrid hydrogels are envisaged herein, and usually referred to as hydrogel systems that possess components from at least two distinct classes of molecules, for example, synthetic polymers and biological macromolecules, interconnected either covalently or non-covalently. Compared to synthetic polymers, proteins and protein modules have well defined and homogeneous structures, consistent mechanical properties, and cooperative folding/unfolding transitions. The protein fiber or multimers of the invention used in said hybrid hydrogel may impose a level of control over the structure formation at the nanometer level; the synthetic part may contribute to the biocompatibility of the hybrid material in certain biomedical applications. By optimizing the amino acid sequence, i.e. by applying engineered Ena proteins, responsive hybrid hydrogels tailor made fora specific application may be designed, Potential applications of different types of hydrogels include tissue engineering, synthetic extracellular matrix, implantable devices, biosensors, separation systems, materials controlling the activity of enzymes, phospholipid bilayer destabilizing agents, materials controlling reversible cell attachment, nanoreactors with precisely placed reactive groups in three-dimensional space, smart microfluidics with responsive hydrogels, and energy-conversion systems.

A final aspect of the invention relates to methods for producing said self-assembling protein subunits, multimers, in vitro or in vivo/in cellulo produced protein fibers, or further to produce ‘arrested’ Ena proteins, engineered forms of Ena proteins, multimers and fiber, and produce modified surfaces of the present invention. The method to produce said protein subunit monomers or self-assembled multimers is a recombinant or in vitro process comprising the steps of:
a) Recombinant expression of the chimeric gene as described herein in a cell, to obtain cells wherein the protein subunits or multimers of the invention are present in the cytosol, optionally encoding engineered Ena protein comprising a heterologous N- or C-terminal tag, and optionally
b) purifying or isolating said proteins or multimers from said modified cell, for instance by cell lysis and separation.

One embodiment relates to said method wherein the protein subunit of the chimeric gene expressed in said cell may be an engineered protein subunit or engineered Ena protein, or may be more than one chimeric construct providing for the expression of one or more wild type Ena proteins and/or different forms of engineered protein subunits of the invention.

Another embodiment relates to said method wherein the purification in step b) comprises the steps of isolation and solubilization of inclusion bodies, refolding of solubilized protein subunits, and purification of refolded protein multimers. Further purification methods for instance using affinity chromatography, ion exchange chromatography, gel filtration, or further alternatives are known to the skilled person.

In another embodiment, the protein subunit, as described herein, in particular an (engineered) Ena protein subunit, encoded by the chimeric gene used in said method to express recombinantly in a cell comprises a heterologous N- or C-terminal tag. Said N- or C-terminal tag may result in production of protein subunits that are still capable to self-assemble into multimers, but due to a non-natural presence of said N- or C-terminal tag, steric hindrance arrests these protein subunits or multimers in further fiber formation or ‘outgrowth’. Most preferable said heterologous N- or C-terminal tag is at least 1-5, 6, 7, 9 or at least 15 amino acids to result in arrested or hampered fiber formation or blocking or retarding of epitaxial growth. Said heterologous N- or C-terminal tag may be an affinity tag, as described herein.

Another embodiment relates to a method to recombinantly produce the protein fiber in a host cell, comprising the steps of:
a) Expression of the chimeric gene in a cell, or using the host cell comprising the Ena protein subunit or multimer as described herein, and
b) Optionally, isolate the self-assembled protein fibers by lysis the cells.

wherein the nucleic acid encoding said self-assembling protein subunit or the Ena protein does not provide for a heterologous N- or C-terminal tag. By recombinantly expressing tag-free or non-sterically hindered Ena proteins, the spontaneous self-assembly into fibers into the cytoplasm allows to easily produce S-type like fibers in vivo.

A further embodiment relates to the in vitro method for producing a protein fiber or engineered protein fiber according to the invention, comprising the steps of:
a) expression of the chimeric gene as described herein in a cell, to obtain cells wherein the protein subunits or multimers of the invention are present, wherein said protein subunits comprise a cleavable heterologous N- or C-terminal tag,
b) purifying said proteins or multimers from said cell,
c) cleavage of the N- or C-terminal tag to result in multimers for covalently connecting to each other to form a fiber.

Alternatively, said protein fiber is produced by said method wherein step b) and c) are reversed. A cleavable tag is for instance a tag with a proteolytic cleavage site, or a cleavable tag as known by the skilled person.

Another embodiment further provides for a method to produce a modified surface as disclosed herein, comprising the steps of the method for producing and purifying the fiber, multimer or engineered forms thereof, followed by a further step of covalently attaching the protein, multimer or fiber to surface, which may be biological or artificial surface.

Finally, there are numerous applications as touched upon already herein for said Ena protein or engineered Ena protein subunit-derived assemblies as next-generation biomaterials in different fields, such as the biomedical and biotechnological areas. So, the use and utility of said nanomaterials is endless.

It is to be understood that although particular embodiments, specific configurations as well as materials and/or molecules, have been discussed herein for methods, and products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Bacillus cereus NVH 0075/95 Show Endospore Appendages of Two Morphological Types

Endospores formed by Bacillus and Clostridium species frequently carry surface-attached feather-, ribbon- or pilus-like appendages (Driks, 2007), the role of which has remained largely enigmatic due to the lack of molecular annotation of the pathways involved in their assembly. Half a century following their first observation (Hachisuka and Kuno, 1976; Hodgikiss, 1971), we herein employ high resolution de novo structure determination by cryoEM to structurally and genetically characterize the appendages found on B. cereus spores.

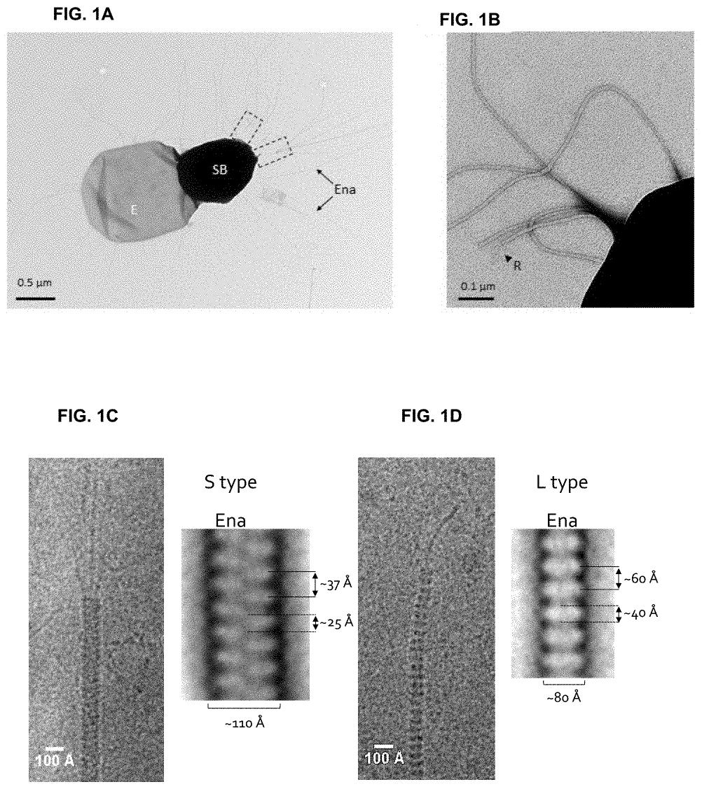

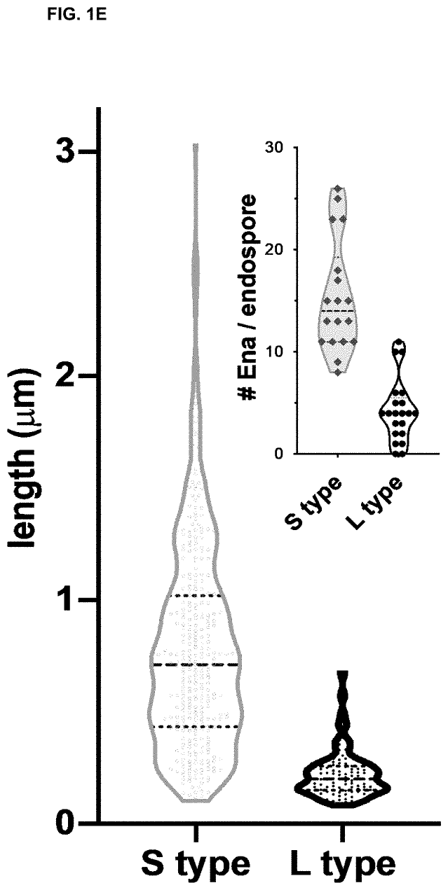

Negative stain EM imaging of B. cereus strain NVH 0075/95 showed typical endospores with a dense core of ˜1 urn diameter, tightly wrapped by an exosporium layer that on TEM images emanates as a flat 2-3 μm long saclike structure from the endospore body (FIG. 1A). The endospores showed an abundance of micrometer-long appendages (Ena) (FIG. 1A). The average endospore counted 20-30 Enas ranging from 200 nm to 6 μm in length (FIG. 1E), with a median length of approximately 600 nm. The density of Enas appeared highest at the pole of the spore body that lies near the exosporium. There, Enas seem to emerge from the exosporium as individual fibers or as a bundle of individual fibers that separates a few tens of nanometers above the endospore surface (FIGS. 1B and 7B). Closer inspection revealed that the Enas showed two distinct morphologies (FIG. 1 C, D). The main or “Staggered-type” (S-type) morphology represents approximately 90% of the observed fibers. S-type Enas have a width of ˜110 Å and give a polar, staggered appearance in negative stain 2D classes, with alternating scales pointing down to the spore surface. At the distal end, S-type Enas terminate in multiple filamentous extensions or “ruffles” of 50-100 nm in length and ˜35 Å thick (FIG. 1C). The minor or “Ladder-like” (L-type) Ena morphology is thinner, ˜80 Å in width, and terminates in a single filamentous extension with dimensions similar to ruffles seen in S-type fibers (FIG. 1D). L-type Enas lack the scaled, staggered appearance of the S-type Enas, instead showing a ladder of stacked disk-like units of ˜40 Å height. Whereas S-type Enas can be seen to traverse the exosporium and connect to the spore body, L-type Enas appear to emerge from the exosporium (FIG. 7A). Both Ena morphologies co-exist on individual endospores (FIG. 7C). Neither Ena morphology is reminiscent of sortase-mediated or type IV pili previously observed in Gram-positive bacteria (Mandlik et al., 2008; Melville and Craig, 2013). In an attempt to identify their composition, shear force extracted and purified Enas were subjected to trypsin digestion for identification by mass spectrometry. However, despite the good enrichment of both S- and L-type Enas, no unambiguous candidates for Ena were identified amongst the tryptic peptides, which largely contained contaminating mother cell proteins, EA1 S-layer and spore coat proteins. Attempts to resolve the Ena monomers by SDS-PAGE were unsuccessful, including strong reducing conditions (up to 200 mM β-mercaptoethanol), heat treatment (100° C.), limited acid hydrolysis (1 h 1M HCl), or incubation with chaotropes such as 8M urea or 6M guanidinium chloride. Ena fibers also retained their structural properties upon autoclaving, desiccation or treatment with proteinase K (FIG. 7C).

We found that B. cereus Enas come in two main morphologies: 1) staggered or S-type Enas that are several micrometer long and emerge from the spore body and traverses the exosporium, and 2) smaller, less abundant ladder- or L-type Enas that appears to directly emerge from the exosporium surface.

### Example 2. Cryo-EM of Endospore Appendages Identifies their Molecular Identity

To further study the nature of the Enas, fibers purified from B. cereus NVH 0075/95 endospores were imaged by cryogenic electron microscopy (cryo-EM) and analysed using 3D reconstruction. Isolated fibers showed a 9.4:1 ratio of S- and L-type Enas, similar to what was seen on endospores. Boxes with a dimension of 300×300 pixels (246×246 Å²) were extracted along the length of the fibers, with an inter-box overlap of 21 Å, and subjected to 2D classification using RELION 3.0 (Zivanov et al., 2018). Power spectra of the 2D class averages revealed a well-ordered helical symmetry for S-type Enas (FIG. 2A, B), whereas L-type Enas primarily showed translational symmetry (FIG. 1D). Based on a helix radius of approximately 54.5 Å, we estimated layer lines Z′ and Z in the power spectrum of S-type Enas to have a Bessel order of −11 and 1, respectively (FIG. 2A, B). In the 2D classes holding the majority of extracted boxes the Bessel order 1 layer line was found at a distance of 0.02673 Å⁻¹ from the equator, corresponding to a pitch of 37.4 Å, in good agreement with spacing of the apparent ‘lobes’ seen also by negative stain (FIGS. 1C, 2B and 7). The correct helical parameters were derived by an empirical approach in which a systematic series of starting values for subunit rise and twist were used for 3D reconstruction and real space Bayesian refinement using RELION 3.0 (He and Scheres, 2017). Based on the estimated Fourier-Bessel indexing, input rise and twist were varied in the range of 3.05-3.65 Å and 29-35 degrees, respectively, with a sampling resolution of 0.1 Å and 1 degree between tested start values. This approach converged on a unique set of helical parameters that resulted in 3D maps with clear secondary structure and identifiable densities for subunit side chains (FIG. 2C). The reconstructed map corresponds to a left-handed 1-start helix with a rise and twist of 3.22937 Å and 31.0338 degrees per subunit, corresponding to a helix with 11.6 units per turn (FIG. 2D). After refinement and postprocessing in RELION 3.0, the map was found to be of resolution 3.2 Å according to the FSC_{0.143} criterion. The resulting map showed well defined subunits comprising an 8-stranded β-sandwich domain of approximately 100 residues (FIG. 2E). The side chain density was of sufficient quality to manually deduce a short motif with the sequence F-C-M-V/T-I-R-Y (FIG. 8A). A search of the B. cereus NVH 0075/95 proteome identified two hypothetical proteins of unknown function, encoded by KMP91697.1 (SEQ ID NO:1) and KMP91698.1 (SEQ ID NO: 8) (FIG. 8B). Further inspection of the electron potential map and manual model building of the Ena subunit showed this to fit well with the sequence encoded by KM P91698.1 which is located 15 bp downstream of the KM P91697.1 locus. Both genes encode hypothetical proteins of similar size (117 and 126 amino acids and estimated molecular weights of 12 and 14 kDa, for KM P91698.1 and KM P91697.1, respectively), with 39% pairwise amino acid sequence identity, a shared domain of unknown function (DUF) 3992 and similar Cys patterns. Further downstream of KMP91698.1, on the minus strand, the KMP91699.1 locus (SEQ ID NO:15) encodes a third DUF3992 containing hypothetical protein, of 160 amino an estimated molecular weight of 17 kDa. As such, KMP91697.1, KMP91698.1 and KMP91699.1 are regarded to encode candidate Ena subunits, hereafter dubbed Ena1A, Ena1B and Ena1C (FIGS. 8 B,C).

### Example 3. Ena1B Self-Assembles into Endospore Appendage-Like Nanofibers In Vitro

To confirm the subunit identity of the endospore appendages isolated from B. cereus NVH0075/95, we cloned a synthetic gene fragment corresponding to the coding sequence of Ena1B and an N-terminal TEV protease cleavable 6×His-tag into a vector for recombinant expression in the cytoplasm of E. coli (recEna1B depicted in SEQ ID NO:83). The recombinant protein was found to form inclusion bodies, which were solubilized in 8M urea before affinity purification. Removal of the chaotropic agent by rapid dilution resulted in the formation of abundant soluble crescent-shaped oligomers reminiscent of a partial helical turn seen in the isolated S-type Enas (FIG. 8A-E), suggesting the refolded recombinant Ena1B (recEna1B) adopts the native subunit-subunit β-augmentation contacts (FIG. 8E). We reasoned that recEna1B self-assemble into helical appendages arrested at the level of a single turn due to steric hindrance by the 6×His-tag at the subunits Ntc's. Indeed, proteolytic removal of the affinity tag readily resulted in the formation of fibers of 110 Å diameter and with helical parameters similar to S-type Enas, though lacking the distal ruffles seen in ex vivo fibers (FIG. 8F). CryoEM data collection and 3D helical reconstruction was performed to assess whether in vitro recEna1B nanofibers were isomorphous with ex vivo S-type Enas. Real space refinement of helical parameters using RELION 3.0 converged on a subunit rise and twist of 3.43721 Å and 32.3504 degrees, respectively, approximately 0.2 Å and 1.3 degrees higher than found in ex vivo S-type Enas, and corresponding to a left-handed helix with a pitch of 38.3 Å and 11.1 subunits per turn. Apart from the minor differences in helical parameters the 3D reconstruction map of in vitro Ena1B fibers (estimated resolution of 3.2 Å; FIG. 9A, B) was near isomorphous to ex vivo S-type Enas in terms of size and connectivity of the fiber subunits (FIG. 9D). Closer inspection of the 3D cryoEM maps for recEna1B and ex vivo S-type Ena showed an improved side chain fit for Ena1B residues in the former (FIG. 9B, C, D) and revealed regions in the ex vivo Ena maps that showed partial side-chain character of Ena1A, particularly in loop L1, L3, L5 and L7 (FIG. 8B, 9B,C). Although the Ena1B character of the ex vivo maps is dominant, this suggested that ex vivo S-type Enas consist of a mixed population of Ena1A and Ena1B fibers, or that S-type Enas have a mixed composition comprising both Ena1A and Ena1B. Immunogold labelling using sera generated with recEna1A or recEna1B showed subunits-specific labeling within single Enas, confirming these have a mixed composition of Ena1A and Ena1B (FIG. 9E). No staining of S-type Enas was seen with Ena1C serum (FIG. 9E). No systematic patterning or molar ratio for Ena1A and Ena1B could be discerned from immunogold labelling or helical reconstructions with an asymmetric unit containing more than one subunit, suggesting the distribution of Ena1A and Ena1B in the fibers to be random. Apart from a number of side chain densities with mixed Ena1A and Ena1B character, the cryoEM electron potential maps of the ex vivo Enas showed a unique main chain conformation, indicating the Ena1A and Ena1B have near isomorphous folds.

### Example 4. Ena1C Self-Assembles into Heptameric Multimers In Vitro

The wild-type sequence of Ena1C (WP_000802321) was codon optimized for expression in E. coli and ordered as a synthetic gene from Twist Bioscience and subcloned further in the pET28a vector (NcoI-XhoI). The insert was designed to have an N-terminal 6× histidine tag followed by a TEV cleavage site (SEQ ID NO:89: ENLYFQG). Large scale recombinant expression was carried out in phage resistant T7 Express lysY/Iq E. coli strain from NEB. The obtained plasmids (pET28a_Ena1A; pET28a_Ena1B) were used to transform competent cells of C43(DE3). Single colonies were used to start overnight (ON) LB cultures. 10 ml ON culture was used to inoculate 11 LB, 25 mg/ml kanamycin at 37 C. Recombinant expression was induced at OD₆₀₀ of 0.8 by addition of 1 mM IPTG and cultures were left to incubate ON. Cells were pelleted by 15 min centrifugation at 4000 g. The whole-cell pellet was resuspended in denaturing lysis buffer (20 mM Potassium Phosphate, 500 mM NaCl, 10 mM 13-ME, 20 mM imidazole, 8M urea, pH 7.5) and sonicated on ice. The lysate was centrifuged to separate the soluble and insoluble fractions by centrifugation at 20,000 rpm for 45 min in a JA-20 rotor from Beckman coulter. The cleared lysate was loaded onto a 5 ml HisTrap HP column packed with Ni Sepharose and equilibrated with denaturing lysis buffer. The bound protein was eluted with elution buffer (20 mM Potassium Phosphate, pH 7.5, 8 M Urea, 250 mM imidazole) in a gradient mode (20-250 mM Imidazole) using an AKTA purifier at room temperature. Resulting fractions were analyzed with SDS-PAGE to check for purity. Fractions containing Ena1C were pooled and refolded by means of dialysis (over-night, 100 μl against 1 liter, 3 kDa cutoff) to 20 mM Potassium Phosphate, 10 mM β-ME, pH 7.5. A 5 μl aliquoted of the refolded material was deposited on Formvar/Carbon grids (400 Mesh, Cu; Electron Microscopy Sciences) and stained using 2% (w/v) uranyl acetate.

As shown in FIG. 14B(i), circular discs or rings of nine subunits were formed solely by recombinant expression of Ena1C. In these disks, the lateral interaction of subunits through β-sheet augmentation can be seen to give rise to a 9-bladed β-propeller.

### Example 5. Enas Represent a Novel Family of Gram-Positive Pili

Upon recognizing that native S-type Enas show a mixed Ena1A and Ena1B composition, we continued with 3D cryoEM reconstruction of recEna1B for model building. The Ena subunit consists of a typical jellyroll fold (Richardson, 1981) comprised of two juxtaposed β-sheets consisting of strands BIDG and CHEF (FIG. 2E). The jellyroll domain is preceded by a flexible 15 residue N-terminal extension hereafter referred to as N-terminal connector (‘Ntc’). Subunits align side by side through a staggered β-sheet augmentation (Remaut and Waksman, 2006), where strands BIDG of a subunit i are augmented with strands CHEF of the preceding subunit i−1, and strands CHEF of subunit i are augmented with strands BIDG of the next subunit in row i+1 (FIG. 2E, FIG. 10A, B). As such, the packing in the endospore appendages can be regarded as a slanted β-propeller of 8-stranded β-sheets, with 11.6 blades per helical turn and an axial rise of 3.2 Å per subunit (FIG. 2E). Subunit-subunit contacts in the β-propeller are further stabilized by two complementary electrostatic patches on the Ena subunits (FIG. 10C). In addition to these lateral contacts, subunits across helical turns are also connected through the Ntc's, where the Ntc of each subunit i makes disulphide bond contacts with subunits i−9 and i−10 in the preceding helical turn (FIG. 2E, FIG. 10B). These contacts are made through disulphide bonding of Cys 10 and Cys 11 in subunit i, with Cys 109 and Cys 24 in the strands I and B of subunits i−9 and i−10, respectively (FIG. 2E, 10B). Thus, disulphide bonding via the Ntc results in a longitudinal stabilization of fibers by bridging the helical turns, as well as in a further lateral stabilization in the β-propellers by covalent cross-linking of adjacent subunits. The Ntc contacts lie on the luminal side of the helix, leaving a central void of approximately 1.2 nm diameter (FIG. 10D). Residues 12-17 form a flexible spacer region between the Ena jellyroll domain and the Ntc. Strikingly, this spacer region creates a 4.5 Å longitudinal gap between the Ena subunits, which are not in direct contact other than through the Ntc (FIG. 3C, 8B). The flexibility in the Ntc spacer and the lack of direct longitudinal protein-protein contact of subunits across the helical turns create a large bendiness and elasticity in the Ena fibers (FIG. 3). 2D class averages of endospore-associated fibers show longitudinal stretching, with a change in pitch of up to 8 Å (range: 37.1-44.9 Å; FIG. 3D), and an axial rocking of up to 10 degrees per helical turn (FIG. 3A, B).

Thus, B. cereus endospore appendages represent a novel class of bacterial pili, comprising a left-handed single start helix with non-covalent lateral subunit contacts formed by β-sheet augmentation, and covalent longitudinal contacts between helical turns by disulphide bonded N-terminal connecter peptides, resulting in an architecture that combines extreme chemical stability (FIG. 7) with high fiber flexibility.

Covalent bonding, and the highly compact jellyroll fold result in a high chemical and physical stability of the Ena fibers, withstanding desiccation, high temperature treatment, and exposure to proteases. The formation of linear filaments of multiple hundreds of subunits requires stable, long-lived subunit-subunit interactions with high flexibility to avoid that a dissociation of subunit-subunit complexes results in pilus breakage. This high stability and flexibility are likely to be adaptations to the extreme conditions that can be met by endospores in the environment or during the infectious cycle.

Two molecular pathways are known to form surface fibers or “pili” in Gram-positive bacteria: 1) sortase-mediated pilus assembly, which encompasses the covalent linkage of pilus subunits by means of a transpeptidation reaction catalyzed by sortases (Ton-That and Schneewind, 2004), and 2) Type IV pilus assembly, encompassing the non-covalent assembly of subunits through a coiled-coil interaction of a hydrophobic. N-terminal helix (Melville and Craig, 2013). Sortase-mediated pili and Type IV pili are formed on vegetative cells, however, and to date, no evidence is available to suggest that these pathways are also responsible for the assembly of endospore appendages.

Until the present study, the only species for which the genetic identity and protein composition of spore appendages has been known, is the non-toxigenic environmental species Clostridium taeniosporum, which carry large (4.5 urn long, 0.5 urn wide and 30 nm thick) ribbon-like appendages, which are structurally distinct from those found in most other Clostridium and Bacillus species. C. taeniosporum lacks the exosporium layer and the appendages seem to be attached to another layer, of unknown composition, outside the coat (Walker et al., 2007). The C. taeniosporum endospore appendages consist of four major components, three of which have no known homologs in other species and an orthologue of the B. subtilis spore membrane protein SpoVM (Walker et al., 2007). The appendages on the surface of C. taeniosporum endospores, therefore, represent distinct type of fibers than those found on the surface of spores of species belonging to the B. cereus group.

Our structural studies uncover a novel class of pili, where subunits are organized into helically wound fibers, held together by lateral β-sheet augmentation inside the helical turns, and longitudinal disulphide cross-linking across helical turns. Covalent cross-linking in pilus assembly is known for sortase-mediated isopeptide bond formation seen in Gram-positive pili (Ton-That and Schneewind, 2004). In Enas, the cross-linking occurs through disulphide bonding of a conserved Cys-Cys motif in the N-terminal connector of a subunit i, to two single Cys residues in the core domain of the Ena subunits located at position i−9 and i−10 in the helical structure. As such, the N-terminal connectors form a covalent bridge across helical turns, as well as a branching interaction with two adjacent subunits in the preceding helical turn (i.e. i−9 and i−10). The use of N-terminal connectors or extensions is also seen in chaperone-usher pili and bacteroides Type V pili, but these system employ a non-covalent fold complementation mechanism to attain long-lived subunit-subunit contacts, and lack a covalent stabilization (Sauer et al., 1999; Xu et al., 2016). Because in Ena the N-terminal connectors are attached to the Ena core domain via a flexible linker, the helical turns in Ena fibers have a large pivoting freedom and ability to undergo longitudinal stretching. These interactions result in highly chemically stable fibers, yet with a large degree of flexibility. Whether the stretchiness and bendiness of Enas are functionally important is yet unclear. Of note, in several chaperone-usher pili, a reversible spring-like stretching provided by helical unwinding and rewinding of the pili has been found important to withstand shear and pulling stresses exerted on adherent bacteria (Miller et al., 2006); (Fallman et al., 2005). Possibly, the longitudinal stretching seen in Ena may serve a similar role.

### Example 6. The Ena1 Coding Region for S-Type Enas

In B. cereus NVH 0075/95 Ena1A, Ena1B and Ena1C are encoded in a genomic region flanked upstream by dedA (genbank: KMP91696.1) and a gene encoding a 93-residue protein of unknown function (DUF1232, genbank: KMP91696.1) (FIG. 4A). Downstream, the ena-gene cluster is flanked by a gene encoding an acid phosphatase. Within the ena-gene cluster, ena1A and ena1B are found in forward, and ena1C in reverse orientation, respectively (FIG. 4A). PCR analysis of NVH 0075/95 cDNA made from mRNA isolated after 4 and 16 h of culture, representative for vegetative growth and sporulating cells, respectively, indicated ena1A and ena1B are co-expressed from a bicistronic transcript during sporulation but not during vegetative growth (FIG. 4B). A weak amplification signal was observed in vegetative cells when the forward primer was located in dedA upstream of ena1A and the reverse primer was located within the ena1B (FIG. 4B, lane 2) suggesting that some enaA and enaB is coexpressed with dedA. This was observed in vegetative cells or very early in sporulation but not during later sporulation stages, and may represent a fraction of improperly terminated dedA mRNA. Quantitative-Real time PCR analysis showed increased expression of ena1A, ena1B and ena1C in sporulating cells compared to vegetative cells (FIG. 4B).

Typical Ena filaments have, to the best of our knowledge, never been observed on the surface of vegetative B. cereus cells indicating that they are endospore-specific structures. In support of that assumption, qRT-PCR analysis NVH 0075/95 demonstrated increased ena1A-C transcript during sporulation, compared to vegetative cells. A transcriptional analysis has previously been performed for B. thuringiensis serovar chinensis CT-43 determining transcription at 7 h, 9 h, 13 h (30% of cells undergoing sporulation) and 22 h after inoculation (Wang et al., 2013). It is difficult to directly compare expression levels of ena1A, B and C in B. cereus NVH 0075/95 with the expression level of ena2A-C in B. thuringiensis serovar chinensis CT-43 (CT43_CH0783-785) since the expression of the latter strain was normalized by converting the number of reads per gene into RPKM (Reads Per Kilo bases per Million reads) and analyzed by DEGseq software package, while the present study determines the expression level of the ena genes relative to the house keeping gene rpoB. However, both studies indicate that enaA and enaB are only transcribed during sporulation. By searching a separate set of published transcriptomic profiling data we found that ena2A-C also are expressed in B. antracis during sporulation (Bergman et al., 2006), although Enas have not previously been reported from B. anthracis spores.

CryoEM maps and immuno-gold TEM analysis of ex vivo S-type Enas indicated these contain both Ena1A and Ena1B (FIG. 9B-D). To determine the relative contribution of Ena1 subunits to B. cereus Enas we made individual chromosomal knockouts of ena1A, ena1B, as well as ena1C and investigated their respective endospores by TEM. All ena1 mutants made endospores of similar dimensions to WT and with intact exosporium (FIG. 5A, FIG. 11). Both the ena1A and ena1B mutant resulted in endospores completely lacking S-type Enas, in agreement with the mixed content of ex vivo fibers. Also the ena1C mutant resulted in the loss of S-type Ena on the endospores (FIG. 5A), even though staining with anti-Ena1C serum did not identify the presence of the protein inside S-type Enas (FIG. 9D). All three mutants still showed the presence of L-type Enas, of similar size and number density as WT endospores, although statistical analysis does not rule out L-type Enas to have a slight increase in length in the ena1B and ena1C mutants (length p=0.003 and <0.0001, resp.) (FIG. 5B). Thus, Ena1A, Ena1B and Ena1C are mutually required for in vivo S-type Ena assembly, but not for L-type Ena assembly. Complementation of the ena1B mutant with a low copy plasmid (pMAD-I-Scel) containing ena1A-ena1B restored S-type Ena expression. Plasmid-based expression of these subunits resulted in an average ˜2-fold increase in the number of S-type Enas per spore, and a drastic increase in Ena length, now reaching several microns (FIG. 5A, B, FIG. 11D). Thus, the number and length of S-type Enas depend on the concentration of available Ena1A and Ena1B subunits. Notably, several endospores overexpressing Ena1A and Ena1B appeared to lack an exosporium or showed the entrapment of S-type Enas inside the exosporium (FIG. 11C, D). This demonstrates that S-type Enas emanate from the spore body, and that a disbalance in the concentration or timing of ena expression can result in mis-assembly and/or mislocalization of endospore surface structures. Contrary to S-type Enas, close inspection of the WT and mutant endospores suggests that L-type Enas emanate from the surface of the exosporium rather than the spore body. The molecular identity of the L-type Ena, or the single or multiple terminal ruffles seen, respectively, in L- and S-type Enas could not be confirmed in present study.

### Example 7. Phylogenetic Distribution of the ena1A-C Genes

To investigate the occurrence of ena1A-C within the B. cereus s.l. group and other relevant species of the genus Bacillus, pairwise tBLASTn searches for homologues of ena1A-C were performed on a database containing all available closed, curated Bacillus spp. genomes, with the addition of scaffolds for species for which closed genomes were lacking (n=735). Homologues with high coverage (>90%) and amino acid sequencing similarity (>80%) of ena1AB of B. cereus NVH 0075/95 were found in 48 strains including 11 of 85 B. cereus strains, 13 of 119 B. wiedmannii strains, 14 of 14 B. cytotoxicus strains, one of one B. luti (100%) strain, 3 of 6 B. mobilis strains, 3 of 33 B. mycoides strains, 1 of 1 B. tropics strain and both B. paranthracis strains analyzed. Of these strains, only 31 also carried a gene encoding a homolog with high sequence identity and coverage to Ena1C of B. cereus NVH 0075/95 (FIG. 6). All investigated B. cytotoxicus genomes (14/14) encoded hypothetical Ena1A and Ena1B proteins, but only 12/14 encoded an Ena1C ortholog, which showed only a moderate amino acid conservation compared to the Ena1C of B. cereus NVH 0075-95 (mean 63.9% amino acid sequence identity) (FIG. 6, FIG. 11).

Upon searching for Ena1A-C homologs in B. cereus group genomes, a candidate orthologous gene cluster encoding hypothetical EnaA-C proteins was discovered. These three proteins had, respectively, an average of 59.3±0.9%, 43.3±1.6% and 53.9±2.2% amino acid sequence identity with Ena1A, Ena1B and Ena1C of B. cereus NVH0075-95, and shared gene synteny (FIG. 6B). The orthologous ena gene cluster was named ena2A-C. Except for B. subtilis (n=127) and B. pseudomycoides (n=8), all genomes analyzed (n=735) carried either ena1 (n=48) or the ena2 (n=476) gene cluster. Ena1A-C or the ena2A-C were never present simultaneously and no chimeric ena1A-C/2A-C clusters were discovered among the genomes analyzed (FIG. 6). In addition to the main split between Ena1A-C and Ena2A-C in the protein trees, distinct sub-clusters were seen among Ena1A, Ena1B and, especially, Ena1C sequences (FIG. 11). The Ena1A sequences separated into two main sub-clusters: one present in the majority of B. cytotoxicus strains and another found in B. wiedmanni and B. cereus strains (FIG. 11A). More variation was evident for EnaB proteins: Ena1B sequences formed two clusters; one containing B. cereus and B. wiedmannii isolates, and the other with B. cytotoxicus (FIG. 11). Also, a separate sub-cluster of Ena2B proteins was seen (FIG. 11), containing isolates of B. mycoides, B. cereus, B. thuringiensis, B. pacificus, and B. wiedmannii that shared around ˜78% and ˜48% sequence identity with the remainder of Ena2B and Ena1B, respectively, EnaC was the most variable of the three proteins: Ena1C formed a monophyletic Glade containing isolates of B. wiedmanni, B. cereus, B. anthracis, B. paranthracis, B. mobilis, B. tropicus, and B. luti, but had considerable sequence variation in species and strains carrying Ena2AB as well as in subset of strains carrying Ena1AB.

The ena2A-C homo- or orthologues were much more common among B. cereus group strains than the ena1A-C genes; all investigated B. toyonensis (n=204), B. albus (n=1), B. bombysepticus (n=1), B. nitratireducens (n=6), B. thuringiensis (n=50) genomes and in the majority of B. cereus (87%, 74/85), B. wiedmannii (105/119, 89.3%), B. tropicus (71%, 5/7,) and B. mycoides (91%, 30/33) had the Ena2A-C form of the protein (FIG. 6). No ena orthologs were found in B. subtilis (n=127) or B. pseudomycoides (n=8) genomes or in any other genomes outside the B. cereus group except for three misclassified Streptococcus pneumoniae genomes (GCA_001161325, GCA_001170885, GCA_001338635) and one misclassified B. subtilis genome (GCA_004328845). These genomes and the B. subtilis were re-classified as B. cereus when re-analyzed with three different methods for taxonomic classification (Masthree, 7-lociMLST and Kraken, see Methods). The genomes of a few Peanibacillus spp. strains had genes encoding hypothetical proteins with a low level of amino acid sequence similarity to Ena1A-C, and genes encoding hypothetical proteins with some similarity to Ena1A and B were also found in the genome of a Cohnella abietis strain (GCF_004295585.1). These hits outside of Bacillus genus was in the DUF3992 domain of these genes, which is found in Anaeromicrobium, Cochnella, and of the order Bacillales.

A few genomes had deviations in the ena-gene clusters compared to other strains of their species. Two of three B. mycoides strains (GCF_007673655 and GCF_007677835.1) lacked the ena1C allele downstream of the ena1A-B operon (data not shown). However, potential ena1c orthologs encoding hypothetical proteins with 50% identity to Ena1C of B. cereus NVH 0075/95 were found elsewhere in their genomes. One genome annotated as B. cereus (strain Rock3-44 Assembly: GCA_000161255.1) grouped with these strains of B. mycoides (FIG. 6) and shared their ena1A-C distribution pattern with. B. thuringiensis usually carries ena2 gene, but a genome annotated as B. thuringiensis (strain LM1212, GCF_003546665) lacked all ena genes. This strain was nearly identical to the reference strain of B. tropicus, which also lacked both the ena gene clusters.

Our phylogenetic analyses of S-type fibers reveal Ena subunits belonging to a conserved family of proteins encompassing the domain of unknown function DUF3992.

### Example 8. Recombinant Production of Tag-Free Ena1A or Ena1B S-Type Fibers In Vivo

Wild-type sequences of Ena1A (WP_000742049.1) and Ena1B (WP_000526007.1) were codon optimized for E. coli and ordered as synthetic genes from Twist Bioscience and subcloned further in the pET28a vector (NcoI-XhoI). The obtained plasmids (pET28a_Ena1A; pET28a_Ena1B) were used to transform competent cells of C43(DE3). Single colonies were used to start overnight (ON) LB cultures. 10 ml ON culture was used to inoculate 11 LB, 25 mg/ml kanamycin at 37 C. Recombinant expression was induced at OD₆₀₀ of 0.8 by addition of 1 mM IPTG and cultures were left to incubate ON. Cells were pelleted by 15 min centrifugation at 4000 g. Cell pellets were resuspended in 1×PBS, 1 mg/ml lysozyme, 1 mM AEBSF, 50 μM leupeptin, 1 mM EDTA and incubated under active stirring at room temperature for 30 min after which DNAse and MgCl₂ were added to a final concentration of 10 μg/ml and 10 mM, respectively, and incubated for another 30 min. Cell debris was pelleted via centrifugation (15 min, 4000 g). The supernatant was carefully removed and centrifuged for 50 min at 20.000 rpm. Supernatants were decanted and pellets were brought back into suspension (1×PBS). The resulting suspension was diluted five-fold in miliQ, deposited on Formvar/Carbon grids (400 Mesh, Cu; Electron Microscopy Sciences) and stained using 2% (w/v) uranyl acetate. TEM analysis revealed the presence of micrometer long fibers with a diameter of 10-11 nm. 2D classification of boxed fiber segments confirms the S-type nature of the observed fibers as shown in FIG. 12.

### Example 9. Biological Role of Ena Proteins: Prospects

Without knowledge on the function of Enas, we can only speculate about their biological role. The Enas of B. cereus group species resemble pili, which in Gram-negative and Gram-positive vegetative bacteria play roles in adherence to living surfaces (including other bacteria) and non-living surfaces, twitching motility, biofilm formation, DNA uptake (natural competence) and exchange (conjugation), secretion of exoproteins, electron transfer (Geobacter) and bacteriophage susceptibility (Lukaszczyk et al., 2019; Proft and Baker, 2009). Some bacteria express multiple types of pili that perform different functions. The most common function of pili-fibers is adherence to a diverse range of surfaces from metal, glass, plastics rocks to tissues of plants, animals or humans. In pathogenic bacteria, pili often play a pivotal role in colonization of host tissues and function as important virulence determinants. Similarly, it has been shown that appendages, expressed on the surface of C. sporogenes endospores, facilitate their attachment to cultured fibroblast cells (Panessa-Warren et al., 2007). The Enas are, however, not likely to be involved in active motility or uptake/transport of DNA or proteins as they are energy demanding processes that are not likely to occur in the endospore's metabolically dormant state. Enas appear to be a widespread feature among spores of strains belonging to the B. cereus group (FIG. 6), a group of closely related Bacillus species with a strong pathogenic potential (Ehling-Schulz et al., 2019). For most B. cereus group species, the ingestion, inhalation or the contamination of wounds with endospores forms a primary route of infection and disease onset. Enas cover much of the cell surface so that they can be reasonably expected to form an important contact region with the endospore environment, and may be speculated to play a role in the dissemination and virulence of B. cereus species. Our phylogenetic analysis shows a widespread occurrence of Enas in pathogenic Bacilli, and a striking absence in non-pathogenic species such as Bacillus subtilis, a soil-dwelling species and gastrointestinal commensal that has functioned as the primary model system for studying endospores. Ankolekar et al., showed that all of 47 food isolates of B. cereus produced endospores with appendages (Ankolekar and Labbe, 2010). Appendages were also found on spores of ten out of twelve food-borne, enterotoxigenic isolates of Bacillus thuringiensis, which is closely related to B. cereus, and best known for its insecticidal activity (Ankolekar and Labbe, 2010).

The cryo-EM images of ex vivo fibers showed 2-3 nm wide fibers (ruffles) at the terminus of S- and L-type Enas. The ruffles resemble tip fibrilla of P-pili and type 1 seen in many Gram-negatives bacteria of the family Enterobacteriaceae (Proft and Baker, 2009). In Gram-negative pilus filaments, the tip fibrilla provides adhesion proteins with a flexible location to enhance the interaction with receptors on mucosal surfaces (Mulvey et al., 1998). No filaments similar to the ruffles were observed on the in vitro assembled fibers suggesting that their formation require additional components than the Ena1A or Ena1B subunits.

We present the molecular identification of a novel class of spore-associated appendages or pili widespread in pathogenic Bacilli. Future molecular and infection studies will need to determine if and how Enas play a role in the virulence of spore-borne pathogenic Bacilli. The advances in uncovering the genetic identity and the structural aspects of the Enas presented in this work now enable in vitro and in vivo molecular studies to tease out their biological role(s), and to gain insights into the basis for Ena heterogeneity amongst different Bacillus species.

### Example 10. Preparation of Ena Thin Films

After isolation of Ena1B recombinantly produced S-fibers in cellulo, a suspension of Ena1B S-type fibers was prepared by diluting the Ena1B stock solution in miliQ to a final concentration of either 100 mg·mL⁻¹ or 25 mg·mL⁻¹. 50 μl of this Ena1B suspension was drop-cast onto a siliconized cover slip with a diameter of 18 mm and incubated at 60° C. for 1 h. Resulting thin films were either used as is (FIG. 21a) or dislodged from the cover slip for imaging (FIG. 21b-c). Both starting concentrations of Ena1B S-type solutions yielded free-standing, translucent thin films with an approximate thickness of 21 μm (FIG. 21c) and 3.7 μm, respectively.

### Example 11. Preparation of Soft and Reinforced Ena Hydrogels

ENA hydrogel preparation—50 μl of a 100 mg·ml⁻¹ Ena1B S-type fiber suspension was pipetted onto a siliconized coverslip and airdried at 22° C. for 1 h (FIG. 22a). Next, 50 μl miliQ was pipetted onto the dried film and left to rehydrate for 5 min at 22° C. (FIG. 22b) resulting in noticeable reswelling of the thin film. Then, excess liquid was removed using a micropipette revealing the resultant Ena1B hydrogel (FIG. 22c), which was free-standing as illustrated in FIG. 22d.

Reinforced ENA hydrogel preparation—20 μl droplets of a 100 mg·ml⁻¹ Ena1B S-type fiber suspension were dropped into 4 M MgCl₂, 5 M NaCl or 100% (v/v) absolute Ethanol and incubated for 1 h at 22° C. The high viscosity of the ENA droplets prevents mixing of the fiber suspension with the chosen solutions, effectively stabilizing the droplet geometry during the incubation period. The high water activity of the salt or ethanol solution leads to a gradual dehydration of the ENA droplet resulting in the formation of a dense ENA hydrogel. The ENA hydrogel beads were 3× transferred to 1 mL of miliQ for removal of salt or ethanol and left to airdry for 24 h at 22° C. (FIG. 23). ENA hydrogel beads resulting from incubation in either MgCl₂ or NaCl were opaque, whereas ethanol incubation lead to stable, translucent structures.

### Example 12. Recombinantly Produced Ena3A Self-Assembles into 1-Type Fibers

A mature spore from a quadruple Ena-knockout strain (Δena1A-1B-1C-ena3A) derived from B. cereus NM 0095-75 revealed a complete absence of any endospore appendages (FIG. 25c), however, upon transforming this mutant with pENA3A, comprising the Ena3A sequence (SEQ ID NO:49), a phenotypic rescue of L-type fibers took place on the spore surface (FIG. 25d-e).

So, based on the identification of Ena3A as a further member of the Ena protein family, essential and sufficient to form L-type Ena fibers on Bacillus endospores, blast searches and a phylogenetic analyses was performed to provide candidate orthologues of Bacillus cereus Ena3A (as presented in SEQ ID NO:49). Multiple sequence alignment of the identified homologues (SEQ ID NO:50-80) is shown in FIG. 19, and demonstrates that besides all sequences comprising a DUF3992-domain, a conserved N-terminal connector region is present for Ena3 as well.

As a representative family member, the Ena3A protein presented in SEQ ID NO:49 was recombinantly expressed, also called herein ‘recEna3A’, and shown to produce helical, 7-start ladder-like (L-type) fibers with a helical twist of 18.4 degrees, a rise of 44.9 Å, and a diameter of 75 Å. L-type fibers are constructed of vertically stacked Ena3A heptameric rings, that are covalently connected via 7 N-terminal connectors. As shown in FIG. 24, Strand G of the BIDG sheet of each subunit is augmented with strand C of the CHEF β-sheet of the adjacent subunit within each heptameric ring unit. Subunits are covalently cross-linked within each ring via disulphide bonding between Cys21 of subunit i and Cys81 of subunit i+1, and between Cys13 of subunit i and Cys14 of subunit i+1. Inter-ring crosslinking is established via the N-terminal connector (Ntc) which forms a disulphide bond at position Cys8 (i) with Cys20 of subunit j in the neighbouring ring.

The in vitro recombinant production of short Ena3 L-type fibers was obtained by expressing sterically blocked Ena3A, purification of the Ena3A multimers, followed by assembly of L-fibers after co-incubation with TEV protease (FIG. 25a; using the method as described for Ena1B). Alternatively, recombinant expression of an Ena3A without steric block in E. coli resulted in ‘in cellulo’ (also called ‘in vivo’ herein) assembly of long L-fibers in the cytoplasm, followed by isolation of the fibers from the cell culture (FIG. 25b; using method as described herein).

So, the CryoEM structure of the Ena3A L-type fiber subunit of Bacillus cereus strain ATCC_10987 (WP_017562367.1; SEQ ID NO:49) provides the cryo-EM model as shown in FIG. 26 (left panel) showing just three subunits to document lateral and longitudinal contacts in the fiber. The Ena subunits are defined by an 8-stranded β-sandwich fold with a BIDG-CHEF topology, as well as an N-terminal extension peptide referred to as the Ntc, and responsible for the longitudinal covalent contacts in the fibers (FIG. 19). To structurally compare this fold with the homologues as presented in FIG. 19, predicted structures using AlphaFold v2.0 for selected Ena3A homologues WP_049681018.1 (SEQ ID NO: 60) and WP_100527630.1 (SEQ ID NO:75) were matched. For each structure, the root-mean-square-deviation (RMSD) of atomic positions between Cα atom i of each structure and the corresponding Cα atom of the reference structure (cryoEM model of Ena3A: WP_017562367.1, SEQ ID NO: 49) was analysed, as well as the fold similarity score, i.e. the Dali Z-score. Z-scores higher than n/10-4 where n is the sequence length are considered to correspond to highly significant fold similarities (10.1093/bioinformatics/btn507). For n=116, this corresponds to Z=7.6. As a benchmark, we also provide the AlphaFold model of our reference structure Ena3A (WP_017562367.1), demonstrating excellent agreement between the experimental cryoEM structure and the AlphaFold model (RMSD=1.05; Z=12.1). These predictions show that DUF3992 sequences with sequence identities as low as at 61% (WP_100527630.1) to our reference sequence can adopt the same ENA-fold with Ntc present.

Thus, Ena3A subunits can be unambiguously identified based on a HMM profile search, resulting in a DUF3992 classification, followed by de novo structure prediction and comparison with the here disclosed for Ena3A cryoEM structures. A self-assembling Ena subunit will contain the eight-stranded Ena beta-sandwich fold with a Dali Z-score to Ena3A (SEQ ID NO: 49) of 6.5 or higher, and will contain a N-terminal connecter peptide with a Z-N-C(C)-M-C-X motif for disulphide-mediated cross-linking in the Ena fiber, and where Z is Leu, Ile, Val or Phe, N is 1 or 2 residues, C is Cys, M is 10 to 12 amino acids, and X is any amino acid. Self-assembly and fiber formation of candidate Ena subunits is done by recombinant expression in the cytoplasm of E. coli, and negative stain transmission electron visualization of isolated fiber material, as here described in material and methods.

### Example 13.1n Vitro Recombinantly Produced Ena2A Self-Assembles into S-Type Fibers

To confirm that besides Ena1B, and Ena3A, the in vitro recombinant production method is generically applicable to all Enas for their typical fibers formation, the in vitro assembly Ena2A S-type fibers is shown in FIG. 27, as obtained by expressing sterically blocked Ena2A (SEQ ID NO: 145) with N-terminal 6×His-TEV blocker, purification of the Ena2A multimers, followed by assembly of S-fibers after co-incubation with TEV protease (FIG. 27; using the method as described for Ena1B).

Similarly, as a confirmation that the in cellulo or in vivo E. coli production of recombinant Ena fiber is also applicable to further Ena family members as shown for Ena1B and Ena3A, the recombinant expression of an Ena2A without steric block in E. coli resulted in ‘in cellulo’ assembly of S-fibers in the cytoplasm, followed by isolation of the fibers from the cell culture (FIG. 28; using method as described herein).

### Example 14. Ena2C Forms Multimeric Discs In Vitro

As shown in example 4 for Ena1C, multimeric disc-type of structures rather than helical multimers are formed in vitro using recombinant EnaC proteins. To further support this in view of Ena2C, similarly, recombinant Ena2C constituting multimers, as nonameric discs, were generated by expressing sterically blocked Ena2C (as presented in SEQ ID NO:146) with N-terminal 6×His-TEV blocker in E. coli Bl21 C43.

Isolation of the multimers and removal of the blocker by cleavage using TEV protease (as provided in the methods described herein), further resulted in L-type-like filaments, though filaments highly flexible and curving into closed loops (FIG. 29).

### Example 15. The N-Terminal Connector is Essential for Disulphide Cross-Linking of Multimers into Fibers

The atomic model from recEna1B S-type fibers shows that the N-terminal connector (Ntc) of subunit i connects to subunits i−9 and i−10 via disulphide cross-linking. Although lateral, non-covalent contacts do exist between two neighbouring subunits (i−1,i), but these interactions are not expected to be sufficient to form robust fibers. To test that hypothesis, a recEna1BΔNtc (deletion of residues 2-15 of WT Ena1B of SEQ ID NO:8) was cloned and expressed in E. coli. Cells were harvested after overnight induction and deposited directly onto a TEM grid and analysed using ns-TEM (FIG. 30). Short S-type Ena fibers were found in the extracellular medium but exhibited spurious defects, that are classify in rupture (FIG. 30b) and fracture points (FIG. 30c-e). Rupture points occur along straight fiber segments, and likely follow from shear forces that arise from solutal flows during sample deposition and blotting steps. Such frequent rupturing was not observed for WT recEna1B fibers and is indicative of the reduced tensile strength of the recEna1BΔNtc fibers. Fracture points were observed in bent fiber regions when a critical curvature of a local fiber segment is exceeded, yielding a sharp angle α^{crit} between two broken segments. Such fracture points suggest a reduced fiber flexibility for the recEna1BΔNtc fibers in comparison to WT recEna1B fibers. These data support the fact that the N-term connector is essential to form inter-subunit disulphide bridges thereby conferring excellent tensile strength and flexibility to the S-type fibers.

### Example 16. In Cellulo Assembly of Rigid S-Type Fibers is Hampered by recEna1B Expression Containing an N-Terminal Steric Block as Little as 6 Amino Acids in Size

Given the original steric block construct, used for the recombinant expression experiments exemplified herein contained 15 additional amino acids over the native Ena sequence (M-His6-SSG-TEV, MHHHHHHSSGENLYFQ-Ena1B, additional amino acids shown in bold), we made constructs containing smaller steric blocks of only 6 (M-TEV-Ena1B, M-ENLYFQ-Ena1B, wherein Ena1B is SEQ ID NO:8 without N-terminal M) or 9 (M-His6-SSG-Ena1B) additional amino acid residues at the N-terminus (FIG. 31). The recombinant expression of both constructs still allow in cellulo fiber formation, however, the fiber yield is strongly reduced as compared to the expression of Ena1B with a steric block of 15aa. The fibers have a smaller diameter (9-9.5 nm) in ns-TEM compared to WT recEna1B S-type fibers (11-11.5 nm), and exhibit less prominent structural features. Note that the diameter of WT Ena1B fibers measured from the atomic cryoEM model is 9.8-9.9 nm. Hence, diameters derived from ns-TEM images are ‘inflated’ due to the uranyl staining halo that surrounds the fibers. We conclude that steric blocks ranging from 6 to 9 amino acids are less optimal for in vitro or in vivo fiber assembly since these steric blocks do not entirely block fibers formation in cellulo, and do not yield native S-type fibers and therefore lower the ability of Ena1B to self-assemble into fibers.

### Example 17. S-Type Fiber Assembly Applying Engineered Ena1B Protein Constructs

Constructs were designed to introduce an HA-tag (YPYDVPDYA) in the BC, DE, EF and HI loop regions of Ena1B, flanked by BamHI sites. For the DE loop, a second construct containing a FLAG-tag (DYKDDDDK) was designed as well. The FLAG-tag is also flanked by BamHI sites. Clear examples of peptide tag insertion in target loops are shown in the aligned sequences below and in FIG. 32, exhibiting efficient S-type polymerization in cellulo. Western blot analysis of the different engineered fibers, as shown in FIG. 33, demonstrates successful presentation of the linear tags (FLAG and HA) on the surface of the fibers, as well as excellent chemical stability (cfr. marked multimer and fiber bands retained in the stacking gel of the SDS-PAGE; samples were boiled in 1% SDS for 15 min).

Alignment of Ena1B native sequence (SEQ ID NO:8) with engineered Ena1B insertion variants:

| | |
|---|---|
| | 10 20 30 40 50 60 |
| | [symbol] |
| Ena1B | MGNCSTNLSCCANGQKTIVQDKVCIDWTAAATAAIIYADNISQDIYASGYLKVDT-----------S |
| DE_FLAG | MGNCSTNLSCCANGQKTIVQDKVCIDWTAAATAAIIYADNISQDIYASGYLKVDTGS-DYKDDDDKG |
| DE_HA | MGNCSTNLSCCANGQKTIVQDKVCIDWTAAATAAIIYADNISQDIYASGYLKVDTGSYPYDVPDYAG |
| HI HA | MGNCSTNLSCCANGQKTIVQDKVCIDWTAAATAAIIYADHISQDIYASGYLKVDT-----------G |
| | 70 80 90 100 110 120 130 |
| | [symbol] |
| Ena1B | [symbol] TGPVTIVFYSGGVTGTAVETIVVATGSSASFTVRRFDTVTILG...........TAAAETGEFCMTIRYTLS |
| DE_FLAG | [symbol] SGPVTIVFYSGGVTGTAVETIVVATGSSASFTVRRFDTVTILG...........TAAAETGEFCMTIRYTLS |
| DE_HA | [symbol] SGPVTIVFYSGGVTGTAVETIVVATGSSASFTVRRFDTVTILG...........TAAAETGEFCMTIRYTLS |
| HI HA | [symbol] TGPVTIVFYSGGVTGTAVETIVVATGSSASFTVRRFDTVTILGGSYPYDVPDYAGSAAETSEFCMTIRYTLS |

[symbol]

indicates data missing or illegible when filed

Furthermore, engineering of the Ena proteins into Ena split-variants, also allowed to in cellulo assemble S-type Ena fibers, as shown in FIG. 34. The split variants were constructed by providing constructs coding for an N-terminal and C-terminal part of Ena1B split at Ala30, so in its BC loop (see FIG. 15), or alternatively split at Ala100, so in its HI loop, respectively. The split BC construct was generated by cloning a stop codon at Ala30, followed by an extra ribosome binding site (RBS) and new ATG start codon in front of former residue 31 in the construct earlier used for in cellulo expression of Ena1B (i.e. pet28a::Ena1B lacking an N terminal 6×His blocker). The split HI construct was generated by cloning a stop codon at Ala100, followed by an extra ribosome binding site (RBS) and new ATG start codon in front of former in the construct earlier used for in-cellulo expression of Ena1B i.e. pet28a::Ena1B lacking an N terminal 6×His blocker).

Thus, Ena protein subunits can be used as engineered Ena subunits by providing them for recombinant expression as split-proteins, wherein at least the split into two polypeptides are shown here to still be able to undergo fold complementation upon co-expression and subsequently self-assembly into Ena S-type fibers.

### Example 18. Epitaxial Growth of Ena1B S-Type Fibers on Magnetic Beads

Isolated recombinantly produced 6×His_TEV_Ena1B multimers were co-incubated with 100 nm Maleimide Super Mag Magnetic Beads (Raybiotech) in 1×PBS for 3 h at RT with continuous shaking and subjected to 3 rounds of washing in 1×PBS to remove any non-bound, sterically blocked Ena1B multimers. Next, the Ena1B functionalized magnetic beads were co-incubated with rec_6×His_TEV_Ena1B solution and TEV-protease, in 1×PBS for 1 h at RT with continuous shaking, and subjected to 3 rounds of washing in 1×PBS to remove any non-bound rec_6×His_TEV_Ena1B and TEV-protease. Next, 3 μl of the functionalized bead suspension was deposited onto a TEM grid and subjected to nsTEM analysis, revealing the presence of short S-type Ena1B fibers tethered to the surface of the magnetic beads (see expanded view in the right figure panel of FIG. 35).

### Example 19. Non-Covalent Surface Functionalization with S-Type Ena Fibers

Recombinantly produced Ena1B S-type fibers were biotinylated using Biotin-dPEG11-MAL (Sigma-Aldrich) during 1 h at RT in 100 mM Tris pH 7.0, and subjected to 2 rounds of washing with miliQ water to remove any non-bound Biotin-dPEG11-MAL. Next, biotinylated Ena1B S-type fibers were co-incubated with streptavidin-coated gold beads (1.25 μm diameter), deposited onto a TEM grid and subjected to nsTEM analysis. Recorded micrographs demonstrate the successful functionalization of gold beads with S-type fibers, i.e. clear tethering of fibers onto the bead surface (FIG. 36). The Biotin-dPEG11-MAL modifications are directed to the unpaired cysteines accessible at the Ena fiber poles, so that surface tethering specifically occurs via the fiber extremities.

### Example 20. Laterally Reinforced Ena Networks Through Site-Directed Mutagenesis

Solvent exposed threonine residues on the surfaces of Ena1B S-type or Ena3A L-type fibers were substituted with cysteines to serve as covalent, lateral, anchoring points through the formations of inter-fiber disulphide bridges. Each of the recombinantly produced proteins Ena1B T31C, Ena3A T40C and Ena3A T69C expressed and self-assembled well in the E. coli cytoplasm. Extraction of the Ena fibers was performed under oxidative conditions to facilitate S-S formation. nsTEM analysis of subsequently obtained fiber fractions revealed the presence of highly entangled Ena fiber networks, both for the Ena1B as the Ena3A point mutants (FIG. 37b,c,e,f). Ena1B T31C fibers exist as larger bundles of varying diameter (FIG. 37b). Higher magnification imaging of a single bundle resolved the individual S-type fibers to be arranged in a parallel manner along the bundle axis, likely resulting in higher tensile strength. This hierarchy of scales suggests a zipper-like S-S assembly mechanism between neighboring Ena 1B T31C S-type fibers. Conversely, Ena3A T40C or T69C L-type fiber isolates are composed of randomly oriented L-type fibers. In this way, lateral cross-linking of Ena fibers can result in the formation of reinforced Ena ropes or bundles, hydrogels and Ena thin films (FIG. 37).

### Examples 21. Identifying Bacterial Self-Assembling Ena Proteins

Based on the observations and analyses presented herein, the Ena proteins are identified as a novel bacterial family of pili-forming protein subunits, belonging to the bacterial DUF3992 proteins, and containing an N-terminal conserved Cys-containing motif. First, identification of bacterial Ena protein family members is based on the amino acid sequence containing a DUF3992 domain, which can be analysed for adhering to the HMM profile of PFAM13157 as shown in Table 1 (or in the PFAM database: https://pfam.xfam.org/family/PF13157#tabview=tab4), and which contains an N-terminal connector (Ntc) comprising at least one conserved Cys, as presented herein, which corresponds to a conserved motif ZXₙCCXₘC, wherein Z is Leu, Ile, Val, or Phe, n is 1 or 2, and m is between 10 and 12 for Ena1/2 A & B proteins (see FIG. 8B), or corresponds to a conserved motif ZXₙC(C)XₘC for the Ena3 proteins (see FIG. 26).

Second, the structural requirements for a protein to be classified as an Ena protein is unambiguously derivable from its (predicted) fold which may simply be based on its amino acid sequence supplied to a modelling tool, as known in the art, and as compared to the Ena1B cryo-EM reference structure, as presented herein, and as deposited in the Protein Database with entry PDB7A02 (Version 1.0—entry submitted Aug. 6, 2000-released Aug. 24, 2000), wherein the fold similarity score, i.e. the Dali Z score, of the predicted fold is 6.5 or higher, since Z-scores higher then (n/10) minus 4, wherein n is the sequence length as the number of amino acids, are considered to correspond to highly significant fold similarities (Holm et al., 2008; Vol. 24 no. 23 p. 2780-2781; doi:10.1093/bioinformatics/btn507). Alternatively, the Ena3 cryo EM reference structure, as presented herein, can be used for determining the fold similarity, as shown in FIG. 26.

Modelling of protein folds can be done by de novo prediction tools as is for instance performed, but not limited to, currently available sources such as Robetta (https://robetta.bakerlab.org/), or AlphaFold v2.0 (Jumper, et al. 2021, Nature; doi.org/10.1038/s41586-021-03819-2), or by homology based protein modelling as can be performed, for instance but not limited to available tools like SWISS-MODEL (https://academic.oup.com/nar/article/46/W1/W296/5000024), Phyre2 (https://www.nature.com/articles/nprot.2015.053), RaptorX (https://www.nature.com/articles/nprot.2012.085) and other.

For instance, structural comparison of a number of selected Ena candidate orthologues, characterized by the DUF3992 classification and the presence of an N-terminal connector, was performed for each 20 structure (shown in FIG. 38), by providing the root-mean-square-deviation (RMSD) of atomic positions between Cα atom i of each structure and the corresponding Cα atom of the reference structure (cryoEM model of Ena1B—Uniprot: A0A1Y6A695; corresponding to SEQ ID NO:8 as depicted herein—coordinates deposited as PDB7A02 or as provided herein in Table 2), as well as the fold similarity score, i.e. the Dali Z-score. Z-scores higher than (n/10) minus 4, wherein n is the sequence length as the number of amino 25 acids, are considered to correspond to highly significant fold similarities (Holm et al., 2008; Vol. 24 no. 23 p. 2780-2781; doi:10.1093/bioinformatics/btn507). So for instance for a protein based on a sequence with n=117, this corresponds to Z=7.6 or higher providing for a strong fold similarity. For DUF3992-domain containing sequences WP_098507345.1 and WP_017562367.1 (www.ncbi.nlm.nih.gov/protein/), we provide the putative structures as predicted by AlphaFold v2.0. As a benchmark, we also provide the AlphaFold model of our reference structure Ena1B (UniProt. A0A1Y6A695, SEQ ID NO:8), demonstrating excellent agreement between the experimental cryoEM structure and the AlphaFold model (RMSD=0.605; Z=12.4). These predictions show that bacterial DUF3992 sequences with sequence identities as low as 24.2% (WP_041638338.1) to our reference sequence (Ena1B, SEQ ID NO:8) can adopt the same Ena-fold with an Ntc present. For Ena2A (WP_001277540.1; SEQ ID NO:145; 24.2% identity) we showed that it does indeed form Ena multimers and S-type Ena fibers. Thus, Ena subunits can be unambiguously identified based on a HMM profile search (according to Table 1, corresponding for HMM matrix of DUF3992-domain containing proteins), followed by de novo structure prediction and comparison with the here disclosed Ena1B and Ena3A cryoEM structures (FIGS. 38 and 26, resp.). A self-assembling Ena subunit will contain the eight-stranded Ena beta-sandwich fold with a Dali Z-score to Ena1B (or Ena3A) of 6.5 or higher, and will contain a N-terminal connecter peptide with a Z-Xₙ-C(C)-Xₘ-C-X motif for disulphide-mediated cross-linking in the Ena fiber, where Z is Leu, Ile, Val or Phe, n is 1 or 2 residues, C is Cys, (C) is an optional second Cys for Ena3 classification, m is 10 to 12 amino acids, and X is any amino acid. Self-assembly and fiber formation of candidate Ena subunits is determined by recombinant expression in the cytoplasm of E. coli, and negative stain transmission electron visualization of isolated fiber material, as here described in material and methods. Specifically, S-type fiber forming Ena subunits can be recognized as DUF3992-domain containing proteins with predicted structure with a Z-score of 6.5 or higher in comparison with Ena1B structure, as provided herein, and having at least 80% sequence identity to any of the Ena1/2 A & B sequences as shown in SEQ ID NOs: 1-14 or 21 to 37, and containing a Z-Xₙ-C-C-Xₘ-C-X motif in the Ntc, where Z is Leu, Ile, Val or Phe, n is 1 or 2 residues, C is Cys, m is 10 to 12 amino acids, and X is any amino acid, and containing a GX_{2/3}CX₄Y motif at the C-terminus, where G=Gly, X=any amino acid, C=Cys and Y=Tyr. S-type Ena fibers are easily recognized by the staggered zig-zag appearance of the fiber helical turns when observed by negative stain electron microscopy (FIG. 1c). Specifically, L-type fiber forming Ena subunits can be recognized as DUF3992-domain containing proteins with predicted structure with a Z-score of 6.5 or higher in comparison with Ena3A structure, as provided herein, and having at least 80% sequence identity to any of the Ena3 sequences as shown in SEQ ID NOs: 49 to 80, and containing a Z-Xₙ-C-Xₘ-C-X motif in the Ntc, where Z is Leu, Ile, Val or Phe, n is 1 or 2 residues, C is Cys, m is 10 to 12 amino acids, and X is any amino acid, and containing a S-Z-N-Y-X-B motif at the C-terminus, where S=Ser, Z is Leu or Ile, N=Asn, B is Phe or Tyr, and X=any amino acid. L-type Ena fibers are easily recognized by the ladder-like appearance of the stacked rings in the fiber when observed by negative stain electron microscopy (FIG. 1d).

TABLE 1
Hidden Markov model of DUF3992 proteins.

| HMER3/f [3.1b2 \| February 2015] | | | |
|---|---|---|---|
| NAME DUF3992 | | | |
| ACC PF13157.8 | | | |
| DESC Protein of unknown function (DUF3992) | | | |
| LENG 88 | | | |
| ALPH amino | | | |
| RF no | | | |
| MM no | | | |
| CONS yes | | | |
| CS no | | | |
| MAP yes | | | |
| DATE Thu Feb 25 02:51:55 2021 | | | |
| NSEQ 3 | | | |
| EFFN 1.022461 | | | |
| CKSUM 4196650675 | | | |
| GA 22.00 22.00; | | | |
| TC 22.50 22.30; | | | |
| NC 21.90 21.40; | | | |
| BM hmmbuild HMM.ann SEED.ann | | | |
| SM hmmsearch -Z 57096847 -E 1000 --cpu 4 HMM pfamseq | | | |
| STATS | LOCAL MSV | −9.2485 | 0.71845 |
| STATS | LOCAL VITERBI | −9.9928 | 0.71845 |
| STATS | LOCAL FORWARD | −3.4552 | 0.71845 |

| | | | | | | | | | | | | | | | | | | | | | |
|---|---|---|---|---|---|---|---|---|---|---|---|---|---|---|---|---|---|---|---|---|---|
| HMM | A | C | D | E | F | G | H | I | K | L | M | N | P | Q | R | S | T | V | W | Y | |
| | m−>m | m−>i | m−>d | i−>m | i−>i | d−>m | d−>d | | | | | | | | | | | | | | |
| COMPO | 2.49118 | 3.78202 | 3.09631 | 2.82201 | 3.45773 | 2.57594 | 3.98925 | 2.59978 | 3.02602 | 2.60346 | 3.73801 | 3.09921 | 3.47093 | 3.22078 | 3.29250 | 2.64826 | 2.55594 | 2.22580 | 4.74956 | 3.70064 | |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.00000 | * | | | | | | | | | | | | | | |
| 1 | 3.22359 | 4.50006 | 5.05511 | 4.57170 | 3.73264 | 4.64487 | 5.23114 | 1.34102 | 4.46986 | 2.21693 | 3.51863 | 4.76350 | 4.94015 | 4.73049 | 4.65436 | 4.04981 | 3.49080 | 0.95149 | 5.73849 | 4.52745 | 1 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 8.77255 | 0.48576 | 0.95510 | | | | | | | | | | | | | | |
| 2 | 3.02311 | 0.46216 | 4.59771 | 4.42163 | 4.35472 | 3.56473 | 4.99814 | 3.56843 | 4.25669 | 3.46149 | 4.59769 | 4.32384 | 4.29541 | 4.58940 | 4.31766 | 3.29354 | 3.51825 | 3.28671 | 5.68400 | 4.63627 | 2 C - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 8.77255 | 0.48576 | 0.95510 | | | | | | | | | | | | | | |
| 3 | 2.64840 | 4.23938 | 3.92369 | 3.39620 | 2.22701 | 3.64366 | 4.06350 | 2.60064 | 3.31194 | 2.40681 | 3.40073 | 3.64489 | 4.09599 | 3.57100 | 3.56243 | 2.06114 | 2.92943 | 1.84147 | 4.82348 | 3.51950 | 3 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 4 | 2.57171 | 4.67932 | 2.99357 | 2.63103 | 4.29890 | 3.28137 | 3.88002 | 3.72029 | 2.71522 | 3.34079 | 4.16258 | 2.09189 | 2.23520 | 3.05704 | 3.14106 | 2.64917 | 2.01710 | 3.31220 | 5.56541 | 4.24816 | 4 t - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 5 | 2.85483 | 4.25513 | 4.47749 | 3.90493 | 3.15885 | 4.02046 | 4.39584 | 1.73702 | 3.74299 | 2.06740 | 3.20552 | 4.05585 | 4.36046 | 3.94515 | 3.87610 | 3.33598 | 3.09194 | 1.65583 | 2.45569 | 3.57028 | 5 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 6 | 2.53637 | 2.46602 | 2.11051 | 2.69279 | 2.13074 | 3.29004 | 3.89527 | 3.52725 | 2.78395 | 3.19103 | 4.02760 | 3.12704 | 3.84973 | 3.09429 | 3.21559 | 1.87210 | 2.87698 | 3.15695 | 5.45259 | 4.15077 | 6 s - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 7 | 1.36053 | 4.40238 | 3.88196 | 3.44870 | 3.63849 | 3.57638 | 4.31560 | 2.64398 | 3.33857 | 1.71952 | 3.58678 | 3.69898 | 4.13846 | 3.67587 | 3.60904 | 2.97420 | 3.05120 | 2.48119 | 5.27567 | 4.04278 | 7 a - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 2.58477 | 3.61503 | |
| | 0.02248 | 4.20204 | 4.92438 | 0.61958 | 0.77255 | 0.48575 | 0.95510 | | | | | | | | | | | | | | |
| 8 | 1.66572 | 4.33263 | 3.50485 | 3.09556 | 4.21535 | 3.14961 | 4.15842 | 3.55816 | 3.09281 | 3.27541 | 4.12206 | 3.34899 | 2.14161 | 3.40639 | 3.44489 | 2.55588 | 1.80062 | 3.12344 | 5.57017 | 4.33716 | 8 a - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.93518 | 4.58477 | 3.61503 | |
| | 0.02248 | 4.20204 | 4.92438 | 0.61958 | 0.77255 | 0.48576 | 0.95510 | | | | | | | | | | | | | | |
| 9 | 1.79579 | 2.28248 | 4.27561 | 3.79203 | 3.65450 | 3.49653 | 4.42930 | 2.45083 | 3.67572 | 2.59575 | 3.60105 | 3.84241 | 4.09805 | 3.91485 | 3.86568 | 2.89711 | 2.92785 | 1.59322 | 5.22418 | 4.03459 | 9 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 10 | 1.68705 | 4.24937 | 3.73282 | 3.31595 | 3.96711 | 2.01017 | 4.24885 | 3.11108 | 3.29010 | 3.00279 | 3.90960 | 3.49723 | 3.89206 | 3.57591 | 3.59115 | 2.63558 | 2.83760 | 1.79672 | 5.41174 | 4.20161 | 10 a - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 11 | 1.39832 | 4.86506 | 2.77211 | 1.80500 | 4.37733 | 3.34060 | 3.90515 | 3.70652 | 2.76849 | 3.38976 | 4.25799 | 3.03905 | 3.91669 | 3.08925 | 3.20525 | 2.77921 | 3.05645 | 3.34921 | 5.65234 | 4.31715 | 11 a - - - |
| | 2.68618 | 4.42225 | 2.77519 | 2.73123 | 3.46354 | 2.40513 | 3.72494 | 3.29354 | 2.57741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 12 | 2.74092 | 5.12674 | 2.03614 | 2.34715 | 4.46265 | 3.38959 | 3.69517 | 3.90913 | 3.92414 | 3.44774 | 4.24077 | 2.89258 | 3.85962 | 2.82512 | 2.95142 | 2.71305 | 2.12969 | 3.51839 | 5.62212 | 4.23212 | 12 k - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 13 | 1.61992 | 4.33119 | 3.50736 | 3.09870 | 4.21812 | 3.14785 | 4.16108 | 3.56130 | 3.09620 | 3.27849 | 4.12482 | 3.35034 | 2.12704 | 3.40929 | 3.44786 | 2.55470 | 1.86114 | 3.12512 | 5.57283 | 4.34022 | 13 a - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 14 | 3.23925 | 4.51185 | 5.07651 | 4.59343 | 3.71005 | 4.66451 | 5.29543 | 1.02853 | 4.48954 | 2.17811 | 3.49315 | 4.78499 | 4.95181 | 4.74230 | 4.66805 | 4.07118 | 3.50524 | 1.24650 | 5.73186 | 4.52916 | 14 i - - - |
| | 2.68625 | 4.42232 | 2.77469 | 2.73130 | 3.46361 | 2.40520 | 3.72502 | 3.29361 | 2.67748 | 2.69362 | 4.24697 | 2.90288 | 2.73747 | 3.18153 | 2.89808 | 2.37894 | 2.77527 | 2.98525 | 4.58484 | 3.61510 | |
| | 0.50000 | 1.56175 | 1.69444 | 0.67164 | 0.71513 | 0.48576 | 0.95510 | | | | | | | | | | | | | | |
| 15 | 2.91591 | 4.42300 | 4.31122 | 3.98026 | 3.65213 | 3.86217 | 4.75954 | 2.05252 | 3.85010 | 2.31676 | 3.59838 | 4.14786 | 4.41639 | 4.20093 | 2.04929 | 3.37558 | 3.27496 | 0.86993 | 5.45888 | 4.19815 | 17 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02737 | 4.00792 | 4.73027 | 0.61958 | 0.77255 | 0.37740 | 1.15723 | | | | | | | | | | | | | | |
| 16 | 3.20190 | 4.54535 | 4.83807 | 4.33690 | 3.46172 | 4.46525 | 4.96700 | 1.96481 | 4.18396 | 1.42916 | 3.29936 | 4.54931 | 4.77805 | 4.42527 | 2.35263 | 3.85185 | 3.46524 | 1.10574 | 5.43189 | 4.24433 | 18 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 17 | 3.83872 | 5.05453 | 4.75599 | 4.45137 | 2.03021 | 4.51692 | 3.56068 | 3.63612 | 4.20809 | 2.95549 | 4.20093 | 4.18505 | 4.83329 | 4.23857 | 1.24260 | 3.90953 | 4.05723 | 3.53284 | 1.28570 | 1.28043 | 19 y - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 18 | 2.36455 | 2.29903 | 3.56121 | 3.24920 | 4.33410 | 3.09525 | 4.30100 | 3.69178 | 3.25620 | 3.42716 | 4.27965 | 3.42106 | 3.82784 | 3.57476 | 3.56354 | 1.67350 | 1.23897 | 3.20347 | 5.69491 | 4.47013 | 20 t - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 19 | 1.97967 | 5.20663 | 1.81759 | 2.30214 | 4.62013 | 3.32401 | 3.79939 | 4.09402 | 2.70659 | 3.63474 | 4.44469 | 1.94101 | 3.88360 | 2.94968 | 3.23776 | 2.77206 | 3.10185 | 3.67319 | 5.81221 | 4.38180 | 21 d - - - |
| | 2.68618 | 4.42225 | 2.77519 | 2.79123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 20 | 2.87873 | 5.31729 | 2.43781 | 1.69669 | 4.68991 | 2.13959 | 3.80138 | 4.17825 | 2.69740 | 3.69436 | 4.50999 | 1.91312 | 3.90385 | 2.95232 | 3.21804 | 2.81781 | 3.15897 | 3.75841 | 5.85830 | 4.42031 | 22 e - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 21 | 2.68728 | 4.56707 | 3.23554 | 2.10002 | 3.78307 | 3.55919 | 3.86132 | 2.11135 | 2.74598 | 2.75645 | 3.68844 | 3.22333 | 3.98096 | 3.09745 | 3.13969 | 2.83942 | 2.17896 | 2.65835 | 5.20482 | 3.93315 | 23 e - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 22 | 2.74812 | 5.02167 | 2.77658 | 2.43055 | 4.27672 | 3.40492 | 2.39350 | 3.82502 | 2.51211 | 3.37859 | 4.19595 | 2.06869 | 2.42071 | 2.88770 | 2.94948 | 2.74671 | 3.00886 | 3.45519 | 5.50998 | 4.11962 | 24 n - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 2.95510 | | | | | | | | | | | | | | |
| 23 | 2.75288 | 4.39412 | 3.71343 | 3.17549 | 2.26259 | 3.73877 | 3.96803 | 2.68420 | 3.08039 | 2.32317 | 3.38509 | 3.53444 | 4.12145 | 2.25738 | 3.38481 | 3.01953 | 2.99302 | 1.93978 | 4.82837 | 3.49627 | 25 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 2.95510 | | | | | | | | | | | | | | |
| 24 | 2.68783 | 4.71517 | 3.06890 | 2.51718 | 3.83432 | 3.47837 | 2.47287 | 3.34349 | 2.58665 | 2.99989 | 3.86557 | 2.18605 | 3.91890 | 2.97188 | 2.98562 | 2.76876 | 2.93978 | 2.21741 | 5.19798 | 3.84365 | 26 n - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 25 | 2.02949 | 4.35594 | 4.53334 | 4.01074 | 3.63691 | 4.15108 | 4.70962 | 1.49193 | 3.91518 | 2.34015 | 3.49712 | 4.22467 | 4.54262 | 4.17707 | 4.13395 | 3.50494 | 3.21922 | 1.35548 | 5.38719 | 4.18205 | 27 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 26 | 2.70736 | 4.33977 | 3.71368 | 3.20219 | 2.25078 | 3.68438 | 3.96280 | 2.66773 | 3.14467 | 2.46884 | 3.45736 | 2.34250 | 4.09938 | 3.43157 | 3.44483 | 2.98298 | 2.96363 | 1.85277 | 4.78516 | 3.41992 | 28 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 27 | 3.12847 | 4.85787 | 3.86856 | 3.81587 | 4.92689 | 0.36362 | 4.90293 | 4.61964 | 4.04121 | 4.22719 | 5.21474 | 4.03445 | 4.25825 | 4.33991 | 4.22874 | 3.31179 | 3.63501 | 4.08398 | 5.92186 | 5.04434 | 29 G - - - |
| | 2.68518 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 28 | 2.62875 | 4.47014 | 3.76400 | 3.54819 | 4.30313 | 3.29448 | 4.53476 | 3.47311 | 3.49795 | 3.32427 | 4.36329 | 3.69176 | 4.02888 | 3.87837 | 3.73123 | 2.83503 | 0.71520 | 3.12997 | 5.70964 | 4.52376 | 30 t - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 3.48576 | 0.95510 | | | | | | | | | | | | | | |
| 29 | 2.73306 | 4.28357 | 4.06853 | 3.55224 | 3.53629 | 2.27012 | 4.30762 | 1.72769 | 3.46339 | 2.42458 | 3.47127 | 3.80762 | 4.23035 | 3.74450 | 3.71816 | 3.11692 | 3.02022 | 1.62232 | 5.13423 | 3.92022 | 31 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 2.48576 | 0.95510 | | | | | | | | | | | | | | |
| 30 | 2.78067 | 4.28145 | 4.13688 | 3.57575 | 2.15146 | 3.85606 | 4.16786 | 2.47952 | 3.46139 | 2.05286 | 2.20948 | 3.82050 | 4.22637 | 3.69433 | 3.67109 | 3.15893 | 2.15316 | 2.37626 | 4.82675 | 3.58867 | 32 l - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.39801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 31 | 3.07870 | 4.54494 | 4.57309 | 4.25868 | 3.81599 | 2.05557 | 5.01175 | 2.09583 | 4.13241 | 2.45275 | 3.74198 | 4.39275 | 4.60676 | 4.47400 | 4.31181 | 3.58973 | 3.43779 | 0.69031 | 5.65204 | 4.39939 | 33 V - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61583 | |
| | 0.02248 | 4.20204 | 4.92438 | 0.61958 | 0.77255 | 0.48576 | 0.95510 | | | | | | | | | | | | | | |
| 32 | 2.73916 | 4.94420 | 2.96956 | 1.95455 | 4.22315 | 3.48418 | 3.69507 | 3.56290 | 1.85710 | 3.19786 | 4.03442 | 3.01277 | 3.90299 | 2.84379 | 2.78578 | 2.75995 | 2.97034 | 2.32741 | 5.43383 | 4.11203 | 34 k - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 2.95510 | | | | | | | | | | | | | | |
| 33 | 3.05277 | 4.87668 | 3.30794 | 3.01175 | 2.79834 | 3.72917 | 3.73187 | 3.54245 | 3.03805 | 3.08280 | 4.09657 | 2.02849 | 4.20697 | 3.38012 | 3.36939 | 3.12834 | 3.32358 | 3.30325 | 4.39882 | 1.31901 | 35 y - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 8.95510 | | | | | | | | | | | | | | |
| 34 | 3.02147 | 5.65404 | 1.58682 | 1.59677 | 4.94021 | 3.32043 | 3.83653 | 4.47473 | 2.83572 | 3.94222 | 4.77143 | 1.87422 | 3.92893 | 2.99190 | 3.43800 | 2.89872 | 3.30279 | 4.02516 | 6.08986 | 4.56777 | 36 d - - - |
| | 2.68624 | 4.42232 | 2.77526 | 2.73130 | 3.46360 | 2.40482 | 3.72501 | 3.29271 | 2.67747 | 2.69361 | 4.24696 | 2.90353 | 2.73746 | 3.18153 | 2.89807 | 2.37893 | 2.77526 | 2.98525 | 4.58483 | 3.61510 | |
| | 0.22489 | 1.63922 | 4.92438 | 0.67034 | 0.71648 | 0.48576 | 0.95510 | | | | | | | | | | | | | | |
| 35 | 2.78841 | 4.55120 | 3.49911 | 3.06221 | 3.71193 | 3.64536 | 4.04597 | 2.75126 | 2.88261 | 2.61737 | 3.67751 | 3.45483 | 4.11336 | 2.17725 | 3.18441 | 3.00247 | 3.06817 | 1.42113 | 5.22868 | 3.95834 | 39 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 36 | 1.50060 | 4.29177 | 3.57354 | 3.37715 | 4.55627 | 1.16066 | 4.47315 | 3.94229 | 3.50694 | 3.67025 | 2.50812 | 3.48397 | 3.83040 | 3.76780 | 3.79134 | 2.54190 | 2.87174 | 3.35647 | 5.88579 | 4.70603 | 40 g - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 8.77255 | 0.48576 | 0.95510 | | | | | | | | | | | | | | |
| 37 | 2.66909 | 4.33730 | 3.64673 | 3.12465 | 3.29604 | 3.62683 | 3.92629 | 2.76635 | 3.04537 | 2.51399 | 3.47282 | 3.46911 | 2.42851 | 3.35528 | 3.35253 | 2.92261 | 2.92757 | 1.95004 | 4.81805 | 2.35477 | 41 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 8.48576 | 0.95510 | | | | | | | | | | | | | | |
| 38 | 1.50060 | 4.29177 | 3.57354 | 3.37715 | 4.55627 | 1.16066 | 4.47315 | 3.94229 | 3.50694 | 3.67025 | 4.50812 | 3.48397 | 3.83040 | 3.76780 | 3.79134 | 2.54190 | 2.87174 | 3.35647 | 5.88579 | 4.70603 | 42 g - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 39 | 2.82223 | 4.93808 | 2.93503 | 2.64700 | 4.37028 | 3.42237 | 3.89468 | 3.86706 | 2.61440 | 3.42112 | 4.30519 | 3.12487 | 1.51779 | 1.96246 | 2.96841 | 2.86403 | 3.12910 | 3.50677 | 5.59068 | 4.27797 | 43 p - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 40 | 2.61268 | 4.33754 | 3.64361 | 3.13594 | 3.62374 | 3.53678 | 4.05301 | 1.94056 | 3.06437 | 2.59397 | 3.56665 | 3.47320 | 2.43826 | 3.38719 | 3.38626 | 2.86198 | 2.08071 | 2.48644 | 5.12020 | 3.89259 | 44 i - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 41 | 2.77047 | 4.29761 | 4.11317 | 3.56446 | 3.42995 | 3.84291 | 4.28372 | 2.29823 | 3.45345 | 2.24351 | 2.25075 | 3.83023 | 4.25129 | 3.72764 | 3.69749 | 3.16136 | 2.07587 | 1.66998 | 5.06414 | 3.87107 | 45 v - - - |
| | 2.68618 | 4.42225 | 2.77519 | 2.73123 | 3.46354 | 2.40513 | 3.72494 | 3.29354 | 2.57741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 3.48576 | 0.95518 | | | | | | | | | | | | | | |
| 42 | 2.64802 | 4.64972 | 2.19459 | 2.65144 | 3.98921 | 3.44081 | 3.85594 | 3.12073 | 2.74163 | 2.98082 | 3.87608 | 3.12900 | 3.92254 | 3.05796 | 3.17895 | 2.75524 | 2.12855 | 2.04643 | 5.36302 | 4.05937 | 46 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 2.77255 | 0.48576 | 0.95510 | | | | | | | | | | | | | | |
| 43 | 2.76702 | 4.50554 | 3.48796 | 2.12130 | 3.61198 | 3.71442 | 3.99986 | 2.60101 | 2.93437 | 1.90486 | 3.52145 | 3.42154 | 4.10637 | 3.28172 | 3.29239 | 2.99504 | 3.00779 | 1.89051 | 5.15230 | 3.90504 | 47 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 44 | 1.99834 | 4.57663 | 3.15033 | 2.05992 | 2.48049 | 3.49953 | 3.81236 | 3.10213 | 2.73670 | 2.79994 | 3.70646 | 3.16648 | 3.93724 | 3.06206 | 3.14473 | 2.78822 | 2.91390 | 2.85311 | 5.13379 | 3.82376 | 48 a - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 45 | 3.21981 | 4.55899 | 4.86076 | 4.29428 | 3.23494 | 4.47559 | 4.82330 | 2.14924 | 4.15723 | 1.26774 | 2.02655 | 4.51394 | 4.71262 | 4.29535 | 4.28168 | 3.81447 | 3.44964 | 1.60406 | 5.21569 | 4.12248 | 49 l - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 46 | 3.03183 | 5.54263 | 1.55728 | 2.23241 | 4.86844 | 3.31891 | 3.89611 | 4.43853 | 2.92311 | 3.94376 | 4.79956 | 1.35871 | 3.95206 | 3.06962 | 3.51333 | 2.93052 | 3.33712 | 3.99722 | 6.06519 | 2.56121 | 50 n - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 47 | 2.37306 | 4.33792 | 3.39719 | 3.21629 | 4.59065 | 1.22443 | 4.39281 | 4.07861 | 3.39947 | 3.73906 | 4.55304 | 3.39806 | 3.82151 | 3.66311 | 3.71815 | 1.54240 | 2.87972 | 3.44845 | 5.90044 | 4.67961 | 51 g - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 48 | 1.39832 | 4.86506 | 2.77211 | 1.80500 | 4.37733 | 3.34060 | 3.90515 | 3.70652 | 2.76849 | 3.38976 | 4.25799 | 3.03905 | 3.91669 | 3.08925 | 3.20525 | 2.77921 | 3.05645 | 3.34921 | 5.65234 | 4.31715 | 52 a - - |
| | 2.68625 | 4.42232 | 2.77527 | 2.73130 | 3.46361 | 2.40480 | 3.72502 | 3.29361 | 2.67748 | 2.69362 | 4.24697 | 2.90354 | 2.73747 | 3.18153 | 2.89808 | 2.37894 | 2.77469 | 2.98525 | 4.58484 | 3.61510 | |
| | 0.24466 | 1.56176 | 4.92438 | 0.67164 | 0.71513 | 0.48576 | 0.95510 | | | | | | | | | | | | | | |
| 49 | 2.01968 | 4.35521 | 4.52743 | 4.00472 | 3.63599 | 4.14618 | 4.70465 | 1.49958 | 3.90930 | 2.34089 | 3.49686 | 4.21928 | 4.53872 | 4.17143 | 4.12874 | 3.49979 | 3.21690 | 1.35764 | 5.38450 | 4.17932 | 55 v - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 50 | 3.22615 | 4.50031 | 5.06340 | 4.57962 | 3.73318 | 4.65219 | 5.28780 | 1.30550 | 4.47866 | 2.21568 | 3.51788 | 4.77084 | 4.94477 | 4.73782 | 4.66210 | 4.05718 | 3.49278 | 0.97426 | 5.74113 | 4.53110 | 56 v - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.22148 | 4.20204 | 1.69444 | 0.61958 | 0.77255 | 0.48576 | 0.95510 | | | | | | | | | | | | | | |
| 51 | 2.80805 | 5.09162 | 1.53898 | 2.29846 | 4.54146 | 3.24505 | 3.82712 | 4.03909 | 2.78750 | 3.60771 | 4.46450 | 2.84680 | 1.98853 | 3.00645 | 3.30786 | 2.78205 | 3.12395 | 3.63187 | 5.74865 | 4.36056 | 57 d - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02737 | 4.00792 | 4.73027 | 0.61958 | 0.77255 | 0.68789 | 0.69843 | | | | | | | | | | | | | | |
| 52 | 2.67743 | 4.87902 | 1.74298 | 2.36539 | 4.36160 | 3.23556 | 3.80760 | 3.72427 | 2.72937 | 3.39514 | 4.25751 | 2.88665 | 3.82166 | 2.98653 | 3.21853 | 2.69759 | 1.86325 | 3.35303 | 5.63442 | 4.25839 | 58 d - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02737 | 4.00792 | 4.73027 | 0.61958 | 0.77255 | 0.37740 | 1.15723 | | | | | | | | | | | | | | |
| 53 | 2.67470 | 2.56436 | 3.73212 | 3.18617 | 2.26388 | 3.65311 | 3.92843 | 2.75887 | 3.07673 | 2.43694 | 3.41311 | 3.51066 | 4.06242 | 2.26311 | 3.36427 | 2.94413 | 2.92710 | 2.56673 | 4.76714 | 3.43357 | 59 q - - - |
| | 2.68618 | 4.42225 | 2.77519 | 2.73123 | 3.46354 | 2.40513 | 3.72494 | 3.29354 | 2.67741 | 2.69355 | 4.24590 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 54 | 2.36455 | 4.29903 | 3.56121 | 3.24920 | 4.33410 | 3.09525 | 4.30100 | 3.69178 | 3.25620 | 3.42716 | 4.27965 | 3.42106 | 3.82784 | 3.57476 | 3.56354 | 1.67350 | 1.23897 | 3.20347 | 5.69491 | 4.47013 | 60 t - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 55 | 3.26958 | 4.61965 | 4.86579 | 4.35486 | 3.32645 | 4.50047 | 4.92924 | 2.08383 | 4.19148 | 1.01476 | 3.15156 | 4.57787 | 4.78383 | 4.39230 | 4.33938 | 3.88412 | 3.52516 | 1.51944 | 5.34923 | 2.20312 | 61 l - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18346 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 56 | 2.00195 | 4.87983 | 3.02717 | 2.55878 | 4.31782 | 3.41664 | 3.73702 | 3.73786 | 2.41530 | 3.31046 | 4.12683 | 2.11558 | 3.88615 | 2.88539 | 2.06076 | 2.72225 | 2.95907 | 3.36769 | 5.49670 | 4.18110 | 62 a - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 57 | 2.91450 | 5.13953 | 2.64020 | 1.69012 | 4.57757 | 3.39463 | 3.91778 | 4.03203 | 2.77567 | 3.60804 | 4.47520 | 3.00830 | 1.46300 | 3.09636 | 3.22052 | 2.90579 | 3.21684 | 3.65484 | 5.76955 | 4.42552 | 63 p - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 58 | 3.12847 | 4.85787 | 3.86856 | 3.81587 | 4.92689 | 0.36362 | 4.90293 | 4.61964 | 4.04121 | 2.22719 | 5.21474 | 4.03445 | 4.25825 | 4.33991 | 4.22874 | 3.31179 | 3.63501 | 4.08393 | 5.92136 | 5.04434 | 64 G - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 59 | 2.79531 | 5.02860 | 2.68274 | 1.38984 | 4.44580 | 3.37503 | 3.83384 | 3.80154 | 2.66254 | 3.44112 | 4.29834 | 2.97224 | 3.91883 | 2.99845 | 3.11000 | 2.80188 | 2.00493 | 3.44561 | 5.67697 | 4.31442 | 65 e - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 60 | 2.51157 | 4.42942 | 3.53236 | 3.37747 | 4.42739 | 3.15513 | 4.47057 | 4.00207 | 3.47467 | 3.70075 | 4.60668 | 3.53777 | 3.92382 | 3.79918 | 3.74031 | 0.68445 | 3.01878 | 3.46178 | 5.77493 | 4.51693 | 66 S - - - |
| | 2.68618 | 4.42225 | 2.77519 | 2.73123 | 3.46354 | 2.40513 | 3.72494 | 3.29354 | 2.67741 | 2.69355 | 4.24690 | 2.98347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 61 | 2.79077 | 4.78200 | 3.33089 | 2.73978 | 3.98572 | 3.60574 | 3.73387 | 2.25862 | 2.33874 | 2.95213 | 3.85327 | 3.19273 | 3.98679 | 2.14401 | 2.01232 | 2.87585 | 3.00665 | 3.07774 | 5.25851 | 4.00086 | 67 r - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 62 | 2.36958 | 4.33841 | 3.40551 | 3.20128 | 4.57558 | 1.71503 | 4.36955 | 4.05988 | 3.36345 | 3.71710 | 4.52852 | 3.38966 | 3.81767 | 3.63296 | 3.68887 | 1.13492 | 2.87130 | 3.43673 | 5.88331 | 4.65917 | 68 s - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 63 | 3.31040 | 4.61340 | 5.03266 | 4.47132 | 1.94050 | 4.56875 | 4.76845 | 1.62617 | 4.33195 | 1.23471 | 2.99330 | 4.62515 | 4.77077 | 4.38563 | 4.39304 | 3.91430 | 3.53174 | 2.29073 | 5.03064 | 3.80512 | 69 l - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 64 | 2.62875 | 4.47014 | 3.76400 | 3.54819 | 4.30313 | 3.29448 | 4.53476 | 3.47311 | 3.49795 | 3.32427 | 4.36329 | 3.69176 | 4.02888 | 3.87837 | 3.73123 | 2.83503 | 0.71520 | 3.12997 | 5.70964 | 4.52376 | 70 t - - - |
| | 2.68618 | 4.42225 | 2.77519 | 2.73123 | 3.46354 | 2.40513 | 3.72494 | 3.29354 | 2.67741 | 2.59355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 65 | 2.73625 | 4.32360 | 3.83210 | 3.28963 | 3.14993 | 3.74181 | 3.96452 | 2.66587 | 3.16691 | 1.81754 | 3.36933 | 3.59702 | 4.12981 | 3.46869 | 3.43374 | 3.03318 | 2.15260 | 2.50032 | 4.72144 | 2.23746 | 71 l - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 66 | 1.39177 | 4.63356 | 3.22690 | 2.73177 | 4.23078 | 3.32734 | 3.84940 | 3.63558 | 2.57919 | 3.25273 | 4.07872 | 3.15163 | 3.86355 | 3.02477 | 2.00833 | 1.93010 | 2.89415 | 3.24778 | 5.47669 | 4.19401 | 72 e - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 2.20204 | 2.92438 | 0.61958 | 0.77255 | 0.48576 | 0.95510 | | | | | | | | | | | | | | |
| 67 | 2.77468 | 5.20757 | 2.06854 | 2.36078 | 4.53771 | 3.42563 | 3.66586 | 4.00475 | 2.37066 | 3.49613 | 4.27984 | 2.90345 | 3.87249 | 2.09477 | 2.05140 | 2.73293 | 3.00919 | 3.60140 | 5.63010 | 4.24773 | 73 r - - |
| | 2.68618 | 4.42225 | 2.77519 | 2.73123 | 3.46354 | 2.20513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 68 | 3.49841 | 4.80265 | 4.99672 | 4.50089 | 1.78464 | 4.63961 | 4.55847 | 2.43824 | 4.34886 | 0.90904 | 3.02862 | 4.62343 | 4.84086 | 4.38648 | 4.41293 | 4.01538 | 3.72094 | 2.59779 | 4.74574 | 3.30970 | 74 l - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 69 | 2.79610 | 5.25507 | 1.91571 | 2.30872 | 4.58163 | 3.38466 | 3.70325 | 4.06625 | 2.48978 | 3.56438 | 4.35135 | 2.08290 | 3.87155 | 2.83169 | 2.13744 | 2.74427 | 3.04486 | 3.65282 | 5.70656 | 4.29343 | 75 d - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02245 | 4.20204 | 2.92438 | 0.61958 | 0.77255 | 0.48576 | 6.95510 | | | | | | | | | | | | | | |
| 70 | 2.66395 | 4.71633 | 2.17396 | 2.56412 | 3.99023 | 3.43223 | 3.79512 | 2.24880 | 2.67815 | 3.02227 | 3.89249 | 3.06146 | 3.90062 | 2.99164 | 3.12616 | 2.06309 | 2.93047 | 3.00074 | 5.33917 | 4.01997 | 76 s - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 71 | 3.23937 | 4.51221 | 5.07587 | 4.59287 | 3.70921 | 4.66397 | 5.29482 | 1.02504 | 4.48875 | 2.17702 | 3.49242 | 2.78451 | 4.95146 | 2.74150 | 4.65724 | 4.07070 | 3.50541 | 1.25174 | 5.73128 | 4.52860 | 77 i - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 72 | 2.77353 | 5.05084 | 2.82912 | 2.45033 | 4.31899 | 2.30801 | 2.39942 | 3.82917 | 2.43084 | 3.37261 | 4.19006 | 2.97217 | 3.90086 | 2.03711 | 2.83594 | 2.76836 | 3.02043 | 3.46638 | 5.51155 | 4.14480 | 78 q - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 73 | 3.22400 | 4.50009 | 5.05646 | 4.57299 | 3.73276 | 4.64607 | 5.28222 | 1.33563 | 4.47129 | 2.21677 | 3.51853 | 4.76469 | 4.94090 | 4.73169 | 4.65563 | 4.05101 | 3.49111 | 0.95482 | 5.73893 | 4.52805 | 79 v - - - |
| | 2.68618 | 4.42225 | 2.77519 | 2.73123 | 3.46354 | 2.20513 | 3.72494 | 3.29354 | 2.67741 | 2.69355 | 4.24690 | 2.90347 | 2.73739 | 3.18146 | 2.89801 | 2.37887 | 2.77519 | 2.98518 | 2.58477 | 3.61503 | |
| | 0.22148 | 4.20204 | 1.69444 | 0.61958 | 0.77255 | 0.48576 | 0.95510 | | | | | | | | | | | | | | |
| 74 | 2.91591 | 2.42300 | 4.31122 | 3.98026 | 3.65213 | 3.86217 | 4.75954 | 2.05252 | 3.85010 | 2.31676 | 3.59838 | 4.14786 | 4.41639 | 2.20093 | 4.04929 | 3.37558 | 3.27496 | 0.86993 | 5.45888 | 4.19815 | 80 v - - - |
| | 2.68576 | 4.42236 | 2.77530 | 2.73134 | 3.46365 | 2.40523 | 3.72505 | 3.29365 | 2.67751 | 2.69312 | 4.24700 | 2.90357 | 2.73694 | 3.18157 | 2.89811 | 2.37897 | 2.77530 | 2.98529 | 4.58488 | 3.61514 | |
| | 0.30589 | 1.36765 | 4.73027 | 0.97736 | 0.47209 | 0.37740 | 1.15723 | | | | | | | | | | | | | | |
| 75 | 1.96262 | 4.77584 | 2.91459 | 1.91818 | 4.22083 | 3.35673 | 3.80169 | 3.57657 | 2.63324 | 3.23544 | 4.07007 | 3.02874 | 3.86720 | 2.97091 | 3.07743 | 2.69140 | 2.05460 | 3.22612 | 5.49521 | 4.16775 | 84 e - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 76 | 2.38200 | 2.20733 | 3.82352 | 3.36626 | 3.93429 | 3.19504 | 4.23765 | 3.23438 | 3.28437 | 3.00476 | 3.89149 | 3.50842 | 2.07101 | 3.57944 | 3.56704 | 2.60288 | 1.85186 | 2.88002 | 5.36516 | 4.16429 | 85 t - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 77 | 1.86688 | 4.74597 | 3.03466 | 2.62139 | 4.31671 | 2.20130 | 3.82618 | 3.72434 | 1.89632 | 3.32805 | 4.14835 | 3.08112 | 3.86875 | 2.99082 | 2.96644 | 2.68260 | 2.93481 | 3.33138 | 5.53986 | 4.23393 | 86 a - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 78 | 3.12847 | 4.85787 | 3.86856 | 3.81587 | 4.92689 | 0.36362 | 4.90293 | 4.61964 | 4.04121 | 4.22719 | 5.21474 | 4.03445 | 4.25825 | 4.33991 | 4.22874 | 3.31179 | 3.63501 | 4.08398 | 5.92186 | 5.04434 | 87 G - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 2.02248 | 4.20204 | 4.92438 | 0.61958 | 0.77255 | 0.48576 | 0.95510 | | | | | | | | | | | | | | |
| 79 | 2.57720 | 4.60280 | 3.05637 | 2.78386 | 4.30206 | 3.25252 | 4.02283 | 3.69416 | 2.88216 | 3.38632 | 4.24621 | 2.00156 | 3.88680 | 3.23751 | 3.26115 | 2.68796 | 1.38530 | 3.28665 | 5.61412 | 4.30911 | 88 t - - - |
| | 2.58618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 80 | 2.58546 | 4.51432 | 3.22883 | 2.81159 | 3.71715 | 2.22778 | 3.88373 | 3.32967 | 2.84117 | 2.99289 | 3.86590 | 3.21870 | 3.90865 | 3.16750 | 3.22778 | 1.96222 | 2.90539 | 3.01755 | 5.14363 | 2.26123 | 89 s - - - |
| | 2.68624 | 4.41959 | 2.77526 | 2.73130 | 3.46360 | 2.40519 | 3.72501 | 3.29368 | 2.67747 | 2.69361 | 4.24696 | 2.90353 | 2.73746 | 3.18153 | 2.89807 | 2.37893 | 2.77473 | 2.98525 | 4.58483 | 3.61509 | |
| | 0.22148 | 1.65339 | 4.92438 | 0.67010 | 0.71674 | 0.48576 | 0.95510 | | | | | | | | | | | | | | |
| 81 | 2.64079 | 4.54619 | 3.29824 | 2.79073 | 3.82031 | 3.49500 | 3.85568 | 3.04922 | 2.69882 | 2.81321 | 3.73052 | 3.22795 | 3.94700 | 2.16609 | 3.06954 | 2.78551 | 2.13822 | 2.09235 | 5.21626 | 3.94988 | 92 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 82 | 3.12847 | 4.85787 | 3.86856 | 3.81587 | 4.92689 | 0.36362 | 4.90293 | 4.61964 | 4.04121 | 4.22719 | 5.21474 | 4.03445 | 4.25825 | 4.33991 | 4.22874 | 3.31179 | 3.63501 | 4.08398 | 5.92186 | 5.04434 | 93 G - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 2.92438 | 0.61958 | 0.77255 | 0.48576 | 0.95510 | | | | | | | | | | | | | | |
| 83 | 2.75274 | 5.08545 | 2.87021 | 1.86062 | 4.43983 | 3.44296 | 3.67722 | 3.87927 | 2.35174 | 3.40293 | 4.20080 | 2.95837 | 3.88290 | 2.80633 | 2.07691 | 2.06180 | 2.99143 | 3.49888 | 5.55856 | 4.21006 | 94 e - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 84 | 3.10285 | 4.42322 | 4.87204 | 4.32035 | 2.06689 | 2.38927 | 4.73182 | 1.53659 | 4.19242 | 1.93215 | 3.21616 | 4.46464 | 4.67002 | 4.34171 | 4.30114 | 3.73289 | 3.34180 | 1.48466 | 5.13935 | 3.91319 | 95 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 85 | 2.69891 | 1.52195 | 4.52330 | 4.08323 | 3.69964 | 3.65752 | 4.67330 | 2.27675 | 3.91967 | 2.51097 | 3.64240 | 4.07479 | 4.26567 | 4.17983 | 4.07513 | 3.09328 | 3.08436 | 1.50307 | 5.37972 | 4.16575 | 96 v - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 86 | 3.23925 | 4.51185 | 5.07651 | 4.59343 | 3.71005 | 4.66451 | 5.29543 | 1.02863 | 4.48954 | 2.17811 | 3.49315 | 4.78499 | 4.95181 | 4.74230 | 4.66805 | 4.07118 | 3.50524 | 1.24650 | 5.73186 | 4.52916 | 97 i - - - |
| | 2.58618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.02248 | 4.20204 | 4.92438 | 0.61958 | 0.77255 | 0.48576 | 0.95510 | | | | | | | | | | | | | | |
| 87 | 2.60561 | 4.73359 | 3.02877 | 2.59477 | 4.22926 | 3.34673 | 3.80010 | 3.64677 | 2.57970 | 3.25421 | 4.07756 | 3.06407 | 3.85931 | 2.11987 | 2.99117 | 1.97393 | 2.03880 | 3.27251 | 5.48104 | 4.16573 | 98 s - - - |
| | 2.68618 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.22148 | 4.20204 | 1.69444 | 0.61958 | 0.77255 | 0.48576 | 0.95510 | | | | | | | | | | | | | | |
| 88 | 2.67004 | 4.31800 | 3.80838 | 3.35161 | 1.97805 | 3.58071 | 4.03072 | 2.63339 | 3.27152 | 2.31700 | 3.43475 | 3.60420 | 4.08035 | 3.55727 | 3.53512 | 2.94757 | 1.87464 | 2.47722 | 4.75404 | 3.34057 | 99 t - - - |
| | 2.68613 | 4.42225 | 2.77519 | 2.73123 | 3.46354 | 2.40513 | 3.72494 | 3.29354 | 2.67741 | 2.69355 | 4.24690 | 2.90347 | 2.73739 | 3.18146 | 2.89801 | 2.37887 | 2.77519 | 2.98518 | 4.58477 | 3.61503 | |
| | 0.01850 | 3.99906 | * | 0.61958 | 0.77255 | 0.00000 | * | | | | | | | | | | | | | | |

Materials and Methods

Culture of B. cereus and Appendages Extraction

For extraction of Enas the B. cereus strain NVH 0075-95 was plated on blood agar plates and incubated at 37° C. for 3 months. Upon maturation, the spores were resuspended and washed in milli-Q water three times (centrifugation 2400×g at 4° C.). To get rid of various organic and inorganic debris, the pellet was then resuspended in 20% Nycodenz (Axis-Shield) and subjected to Nycodenz density gradient centrifugation where the gradient was composed of a mixture of 45% and 47% (w/v) Nycodenz in 1:1 v/v ratio. The pellet consisting only of the spore cells was then washed with 1M NaCl and TE buffer (50 mM Tris-HCl; 0.5 mM EDTA) containing 0.1% SDS respectively. To detach the appendages, the washed spores were sonicated at 20k Hz±50 Hz and 50 watts (Vibra Cell VC50T; Sonic & Materials Inc.; U.S.) for 30 s on ice followed by centrifugation at 4500×g and appendages were collected in the supernatant. To further get rid of the residual components of spore and vegetative mother cells n-Hexane was added and vigorously mixed with the supernatant in 1:2 v/v ratio. The mixture was then left to settle to allow phase separation of water and hexane. The hexane fraction containing the appendages was then collected and kept at 55° C. under pressured air for 1.5 hrs to evaporate the hexane. The appendages were finally resuspended in mill-Q water for further cryo-EM sample preparation.

Recombinant Expression, Purification and In Vitro Assembly of Ena1B Appendages

Ena1B was codon optimized for expression in E. coli., synthesized and cloned into Pet28a expression vector at Twist biosciences (SEQ ID NO:83). The insert was designed to have a N-terminal 6× histidine tag on Ena1B along with a TEV protease cleavage site (SEQ ID NO:89: ENLYFQG) in between. Large scale recombinant expression was carried out in phage resistant T7 Express lysY/Iq E. coli strain from NEB. A single colony was inoculated into 20 mL of LB and grown at 37° C. with shaking at 150 rpm overnight for primary culture. Next morning 6 L of LB was inoculated with 20 mL/L of primary culture and grown at 37° C. with shaking until the OD₆₀₀ reached 0.8 after which protein expression was induced with 1 mM isopropyl β-D-1-thiogalactopyranoside (IPTG). The culture was incubated for a further 3 hrs at 37° C. and harvested by centrifugation at 5,000 rpm. The whole-cell pellet was resuspended in soluble lysis buffer (20 mM Potassium Phosphate, 500 mM NaCl, 10 mM β-ME, 20 mM imidazole, pH 7.5) and sonicated on ice for lysis. The lysate was centrifuged to separate the soluble and insoluble fractions by centrifugation at 18,000 rpm for 45 min in a JA-20 rotor from Beckman coulter. The pellet was further dissolved in denaturing lysis buffer consisting 8M urea in lysis buffer. The dissolved pellet was then passed HisTrap HP columns packed with Ni Sepharose and equilibrated with denaturing lysis buffer. The bound protein was then eluted out from the column with elution buffer (20 mM Potassium Phosphate, pH 7.5, 8 M Urea, 250 mM imidazole) in a gradient mode (20-250 mM Imidazole) using an AKTA purifier at room temperature. Recombinantly purified Ena1B with intact N terminal 6×HIS tag in denaturing conditions was subjected to buffer exchange with soluble lysis buffer by dialysis button from Hampton. As the N terminal His tag hindered the formation of double disulphide bridge between two monomers, Ena1B assembled into spirals (FIG. 8E). To facilitate self-assembly into filaments the His-tag was cleaved off by TEV protease. Purified Ena1B in denaturing conditions was first dialyzed with a buffer containing 20 mM Hepes, pH 7.0, 50 mM NaCl overnight at 4° C. TEV protease along with 100 mM 6-ME was then added in equimolar ratio and incubated for 2 hrs. at 37° C. This led to the assembly of the Ena1B into long filaments FIG. 8F.

Isolation of Recombinant In Vivo/in Cellulo Ena Fibers from Escherichia coli: [as Exemplified Herein for S-Type Fibers as in FIG. 20; and L-Type Fibers as in FIG. 25]

Inoculate 1 liter of LB, 50 μg/ml kanamycin with 20 mL of an overnight pre-culture of E. coli C43(DE3) pET28a Ena1B or Ena3A, without steric block (i.e. for instance without HIS tag-TEV cleavage site as compared to in vitro assembly method). Incubate in a rotary shaker at 37° C. until mid-exponential phase (OD=0.7-1.0), lower temperature to 25° C. and add 1 mM final isopropyl β-d-1-thiogalactopyranoside. Incubate for 18 h, and harvest cells using a JLA 8.1 rotor at 5.000 rcf and 4° C. Resuspend cell pellets in 1×PBS, 1% (w/v) sodium dodecyl sulfate (SDS) using an overhead stirrer mounted with a propeller style agitator at 2000 rpm. Incubate the cell slurry for 30 min on a magnetic hotplate set to 99° C. while continuously stirring with a magnetic stirrer bar. Transfer homogenized lysate to 50 ml falcon tubes and centrifuge for 30 min at 20.000 rcf in a JLA 14.5 rotor at 20° C. Discard supernatant and resuspend pellets in 1×PBS using a Potter-Elvehjem tissue grinder with radial serrations and centrifuge homogenate for 30 min at 20.000 rcf. Discard supernatant and resuspend pellets in miliQ and centrifuge for 30 min at 20.000 rcf. Redissolve cleared Ena pellets in miliQ to reach desired final concentration.

Ena Treatment Experiments to Test its Robustness

Ex vivo Enas extracted from B. cereus strain NVH 0075-95 (see above) were resuspended in deionized water, autoclaved at 121° C. for 20 minutes to ensure inactivation of residual bacteria or spores, and subjected to treatment with buffer or as indicated below and shown in FIG. 7. To determine Ena integrity upon the various treatments, samples were imaged using negative stain TEM and Enas were boxed and subjected to 2D classification as described below. To test protease resistance, ex vivo Ena were subjected to 1 mg/mL Ready-to-use Proteinase K digestion (Thermo Scientific) for 4 hours at 37° C. and imaged by TEM. To study the effects of desiccation on the appendages, ex vivo Ena were vacuum dried at 43° C. using Savant DNA120 Speedvac Concentrator (Thermo scientific) run for 2 hours at a speed of 2k rpm.

Negative-Stain Transmission Electron Microscopy (TEM)

For visualization of spores and recombinantly expressed appendages by NS-TEM, formvar/carbon coated copper grids with 400-hole mesh from Electron Microscopy Sciences was discharged in a ELMO glow discharger with a plasma current of 4 mA at vacuum for 45 s. 3 μL of sample was applied on the grids and allowed to bind to the support film for 1 min after which the extra liquid was blotted out with Whatman grade 1 filter paper. The grid was then washed three times using three 15 μL drops of milli-Q followed by blotting of extra liquid. The washed grid was kept in 15 μL drops of 2% Uranyl acetate three times with 10 s, 2 s and 1 min long durations with a blotting step in between each dip. Finally, the uranyl acetate coated grids were blotted until drying. The grids were then screened using a 120 kV JEOL 1400 microscope equipped with LaB6 filament and TVIPS F416 CCD camera. 2D classes of the appendages were generated in RELION 3.0. as described later.

Preparation of Cryo-TEM Grids and Cryo-EM Data Collection

QUANTIFOIL® holey Cu 400 mesh grids with 2 μm holes and 1 μm spacing were first glow discharged in vacuum using plasma current of 5 mA for 1 min. 3 μL of 0.6 mg/mL Graphene Oxide (GO) solution was applied onto the grid and incubated 1 min for absorption at room temperature. Extra GO was then blotted out and left for drying using a Whatman grade 1 filter paper. For cryo-plunging, 3 μL of protein sample was applied on the GO coated grids at 100% humidity and room temperature in a Gatan CP3 cryo-plunger. After 1 min of absorption it was machine-blotted with Whatman grade 2 filter paper for 5 s from both sides and plunge frozen into liquid ethane at 180° C. Grids were then stored in liquid nitrogen until the data collection. Two datasets were collected for ex vivo and recEna1B appendages with slight changes in the collection parameters. High resolution cryo-EM 2D micrograph movies were recorded on a JEOL Cryoarm300 microscope automated with Serial EM in counting mode. For the ex vivo grown appendages, the microscope was equipped with a K2 summit detector and had the following settings: 300 keV, 100 mm aperture, 30 frames, 62.5 e⁻/Å², 2.315 s exposure, and 0.82 Å/pxl. For the recEna1B dataset a K3 detector was used instead that had a pixel size of 0.782 Å/pxl, with an exposure of 64.66 e⁻/Å² taken over 61 frames.

Image Processing

MOTIONCORR2 (Zheng et al., 2017) implemented in RELION 3.0 (Zivanov et al., 2018) was used to correct for beam-induced image motion and averaged 2D micrographs were generated. The motion-corrected micrographs were used to estimate the CTF parameters using CTFFIND4.2 (Rohou and Grigorieff, 2015) integrated in RELION 3.0. Subsequent processing used RELION 3.0. and SPRING (Desfosses et al., 2014). For both the datasets, the coordinates of the appendages were boxed manually using e2helixboxer from the EMAN2 package (Tang et al., 2007). Special care was taken to select micrographs with good ice and straight stretches of Ena filaments. The filaments were segmented into overlapping single-particle boxes of dimension 300×300 pxl with an inter-box distance of 21 Å. For the ex vivo Enas a total of 53,501 helical fragments was extracted from 580 micrographs with an average of 2-3 long filaments per micrograph. For the recEna1B filaments, 100,495 helical fragments were extracted from 3,000 micrographs with an average of 4-5 filaments per micrograph. To filter out bad particles multiples rounds of 2D classification were run in RELION 3.0. After several rounds of filtering, a dataset of 42,822 and 65,466 good particles of the ex vivo and recEna1B appendages were selected, respectively.

After running ˜50 iterations of 2D classification well-resolved 2D class averages could be obtained. segclassexam of the SPRING package (Desfosses et al., 2014) was used to generate B-factor enhanced power spectrum of the 2D class averages. The generated power spectrum had an amplified signal-to-noise ratio with well resolved layer lines (FIG. 2B). To estimate crude helical parameters, coordinates and phases of the peaks in the layer lines were measured using the segclasslayer option in SPRING. Based on the measured distances and phases possible sets of Bessel orders were deduced, after which the calculated helical parameters were used in a helical reconstruction procedure in RELION (He and Scheres, 2017). A featureless cylinder of 110 Å diameter generated using relion_helix toolbox was used as an initial model for 3D classification. Input rise and twist deduced from Fourier-Bessel indexing were varied in the range of 3.05-3.65 Å and 29-35 degrees, respectively, with a sampling resolution of 0.1 Å and 1 degree between tested start values. So doing, several rounds of 3D classification were run until electron potential maps with good connectivity and recognizable secondary structure were obtained. The output translational information from the 3D classification was used to re-extract particles and 3D refinement was done taking a 25 Å low pass filtered map generated from the 3D classification run. To improve the resolution of the EM maps multiple rounds of 3D refinement were run. To further improve the resolution Bayesian polishing was performed in RELION. Finally, a solvent mask covering the central 50% of the helix z-axis was generated in maskcreate and used for postprocessing and calculating the solvent-flattened Fourier shell correlation (FSC) curve in RELION. After two rounds of polishing, maps of 3.2 Å resolution according to the FSC_{0.143} gold-standard criterion as well as local resolution calculated in RELION were obtained (FIG. 9A).

Model Building

To improve the connectivity of the asymmetrical units, density modification for cryo-EM tool implemented in PHENIX (Afonine et al., 2018) was used. At first the primary skeleton for a single asymmetric subunit from the density modified map was generated in Coot (Emsley et al., 2010). Primary sequence of Ena1B was manually threaded into the asymmetric unit and fitted into the map taking into consideration the chemical properties of the residues. SSM Superpose option in coot was used to build the helix from a single subunit. The built model was then subjected to multiple rounds real space structural refinement in Phenix, each residue was manually inspected after every round of refinement. Model validation was done in Refmac implemented in Phenix. All the visualizations and images for figures were generated in ChimeraX (Goddard et al., 2018), Chimera (Pettersen et al., 2004), Pymol.

Immunostaining of Enas

Aliquots of purified RecEna1A, RecEna1B and RecEna1C were sent to Davids Biotechnologie GmbH (Germany) for rabbit immunization (28-day SuperFast immunization schedule; A055). Sera were received after one month and used without further affinity purification. For immunostaining EM imaging, 3 μl aliquots of purified ex vivo Enas were deposited on Formvar/Carbon grids (400 Mesh, Cu; Electron Microscopy Sciences), washed with 1×PBS, and incubated for 1 h with 0.5% (w/v) BSA in 1×PBS. After additional washing with 1×PBS, separate grids were incubated for 2 h at 37° C. with 1000-fold dilutions in 1×PBS of anti-Ena1A, anti-Ena1B, and anti-Ena1C sera, respectively. Following washing with 1×PBS, grids were incubated for 1 h at 37° C. with a 2000-fold dilution of 10 nm gold labeled Anti-Rabbit IgG produced in goat, and affinity isolated antibody (G7277-.4ML; Sigma-Aldrich).

Quantitative RT-PCR

Quantitative RT-PCR experiments were performed on isolated mRNA from B. cereus cultures harvested from three independent Bacto media cultures (37° C., 150 rpm) at four, eight, 12 and 16 hrs post-inoculation. RNA extraction, cDNA synthesis and RT-qPCR analysis was performed as essentially described before (Madslien et al., 2014), with the following changes: pre-heated (65° C.) TRIzol Reagent (Invitrogen) and bead beating 4 times for 2 min in a Mini-BeadBeater-8 (BioSpec) with cooling on ice in between. Each RT-qPCR of the RNA samples was performed in triplicate, no template was added in negative controls, and rpoB was used as internal control. Slopes of the standard curves and PCR efficiency (E) for each primer pair were estimated by amplifying serial dilutions of the cDNA template. For quantification of mRNA transcript levels, Ct (threshold cycle) values of the target genes and the internal control gene (rpoB) derived from the same sample in each RT-qPCR reaction were first transformed using the term E^{-Ct}. The expression levels of target genes were then normalized by dividing their transformed Ct-values by the corresponding values obtained for the internal control gene (Duodu et al., 2010; Madslien et al., 2014; Pfaffl, 2001). The amplification was conducted by using StepOne PCR software V.2.0 (Applied Biosystems) with the following conditions: 50° C. for 2 min, 95° C. for 2 min, 40 cycles of 15 s at 95° C., 1 min at 60° C. and 15 s at 95° C. All primers used for RT-qPCR analyses are listed in Table 2. Regular PCR reactions were performed on cDNA to confirm that enaA and enaB were expressed as an operon using the primers 2180/2177 and 2176/2175 and DreamTaq DNA polymerase (Thermo Fisher) amplified in an Eppendorf Mastercycler using the following program: 95° C. for 2 min, 30 cycles of 95° C. for 30 s, 54° C. for 30 s, and 72° C. for 1 min.

Construction of Deletion Mutants

The B. cereus strain NVH 0075/95 was used as background for gene deletion mutants. The ena1B gene was deleted in-frame by replacing the reading frames with ATGTAA (5′-3′) using a markerless gene replacement method (Janes and Stibitz, 2006) with minor modifications. The Δena1B Δena1C double mutant was constructed by deletion of ena1C in the B. cereus strain NVH 0075/95 Δena1B background.

To create the deletion mutants the regions upstream (primer A and B, Table 2) and downstream (primer C and D, Table 2) of the target ena genes were amplified by PCR. To allow assembly of the PCR fragments, primers B and C contained complementary overlapping sequences. An additional PCR step was then performed, using the upstream and downstream PCR fragments as template and the A and D primer pair (Table 2). All PCR reactions were conducted using an Eppendorf Mastercycler gradient and high fidelity AccuPrime Taq DNA Polymerase (ThermoFisher Scientific) according to the manufacturer's instructions. The final amplicons were cloned into the thermosensitive shuttle vector pMAD (Arnaud et al., 2004) containing an additional I-Scel site as previously described (Lindback et al., 2012). The pMAD-I-Scel plasmid constructs were passed through One Shot™ INV110 E. coli (ThermoFisher Scientific) to achieve unmethylated DNA to enhance the transformation efficiency in B. cereus. The unmethylated plasmid were introduced into B. cereus NVH 0075/95 by electroporation (Mahillon et al., 1989). After verification of transformants by PCR, the plasmid pBKJ233 (unmethylated), containing the gene for the I-Scel enzyme, was introduced into the transformant strains by electroporation. The I-Scel enzyme makes a double-stranded DNA break in the chromosomally integrated plasmid. Subsequently, homologous recombination events lead to excision of the integrated plasmid resulting in the desired genetic replacement. The gene deletions were verified by PCR amplification using primers A and D (Table 2) and DNA sequencing (Eurofins Genomics).

Search for Orthologues and Homologues of Ena1

Publicly available genomes of species belonging to the Bacillus s.l. group was downloaded from NCBI RefSeq database (n=735, NCB (https://www.ncbi.nlm.nih.gov/refseq/). Except for strains of particular interest due to phenotypic characteristics (GCA_000171035.2_ASM17103v2, GCA_002952815.1_ASM295281v1, GCF_000290995.1_Baci_cere_AND1407_G13175) and species of which closed genomes were non-existent or very scarce, all assemblies included were closed and publicly available genomes from the curated database of NCBI RefSeq. Assemblies were quality checked using QUAST (Gurevich et al., 2013), and only genomes of correct size (˜4.9-6 Mb) and a GC content of ˜35% were included in the downstream analysis. Pairwise tBLASTn searches were performed (e-value 1e-10, max_hspr 1, default settings) to search for homo- and orthologs of the following query-protein sequences from strain NVH 0075-95: Ena1A (SEQ ID NO:1), Ena1B (SEQ ID NO:87), Ena1C (SEQ ID NO:15). The Ena1B protein sequence (SEQ ID NO:87) used as query originated from an inhouse amplicon sequenced product, while the Ena1A and Ena1C protein sequence queries originated from the assembly for strain NVH 0075-95 (Accession number GCF_001044825.1, protein KMP91697.1 and KMP91699.1, resp. We considered proteins orthologs or homologs when a subject protein matched the query protein with high coverage (>70%) and moderate sequence identity (>30%).

Comparative Genomics of the Ena-Genes and Proteins

Phylogenetic trees of the aligned Ena1A-C proteins were constructed using approximately maximum likelihood by FastTree (Price et al., 2010) (default settings) for all hits resulting from the tBLASTn search. The amino acid sequences were aligned using mafft v.7.310 (Katoh et al., 2019), and approximately-maximum-likelihood phylogenetic trees of protein alignments were made using FastTree, using the JTT+CAT model (Price et al., 2010). All Trees were visualized in Microreact (Argimon et al., 2016) and the metadata of species, and presence and absence for Ena1A-C and Ena2A-C overlaid the figures.

TABLE 2
Cryo-EM model and data statistics

| | Ex vivo | recENA1B | |
|---|---|---|---|
| | S-type Ena | (EMDB-11591) | |
| | (EMDB-11592) | (PDB7A02) | |
| | CryoARM300, | CryoARM300, | |
| | BECM | BECM | |
| Data collection and processing | | | |
| Magnification | 60.000 | 60.000 | |
| Voltage (kV) | 300 | 300 | |
| Electron exposure (e−/Å²) | 62.5 | 64.66 | |
| Defocus range (μm) | −0.5 to −3.5 | −0.5 to −3.5 | |
| Pixel size (Å) | 0.82 | 0.784 | |
| | Helical | Helical | |
| Symmetry imposed | Rise = 3.22937 | Rise = 3.43721 | |
| | Rotation = 31.0338 | Rotation = 32.3504 | |
| Initial particle images (no.) | 53501 | 100495 | |
| Final particle images (no.) | 42822 | 65466 | |
| Map resolution (Å) | 3.2 | 3.05 | |
| FSC threshold | 0.143 | 0.143 | |
| Map resolution range (Å) | | 3.05-3.65 ¹ | |
| Refinement | | | |
| Initial model used | NA | de novo | |
| Model resolution (Å) | NA | 2.81 | |
| FSC threshold | NA | 0.143 | |
| Model resolution range (Å) | | | |
| Map sharpening β factor (Å²) | 25.9 B-iso | 27.4 B-iso | |
| | of density | of density | |
| | modification | modification | |
| Model composition | | | |
| Non-hydrogen atoms | NA | 18699 ² | |
| Protein residues | | 2576 ² | |
| Ligands | NA | 0 | |
| β factors (Å²) | | | |
| Protein | NA | 54.39 | |
| Ligand | NA | NA | |
| R.m.s. deviations | | | |
| Bond lengths (Å) | NA | 0.008 | |
| Bond angles (°) | NA | 0.736 | |
| Validation | | | |
| MolProbity score | NA | 1.93 | |
| Clashscore | NA | 8.07 | |
| Poor rotamers (%) | NA | 0 | |
| Ramachandran plot | | | |
| Favored (%) | NA | 101 | (92%) ³ |
| Allowed (%) | NA | 9 | (8%) ³ |

| | | |
|---|---|---|
| Disallowed (%) | NA | 0 ³ |

¹ Numbers reflect the density modified cryo-EM map calculated using ResolveCryoEM (Terwilliger et al., 2019)
² Numbers reflect a S-type Ena model with 23 Ena1B protomers
³ Numbers for a single Ena1B protomer

Sequence List

>SEQ ID NO: 1: Bacillus cereus NVH 0075-95 383 Endospore appendage (Ena) 1A amino acid sequence
(GenBank Protein ID: KMP91697.1; 126aa)
>SEQ ID NO: 2: GCF_007673655.1_Ena1A 125aa B. mycoides (as on the ncbi database)
>SEQ ID NO: 3: GCF_002251005.2_Ena1A 126aa B. cytotoxicus
>SEQ ID NO: 4: GCF_001884105.1_Ena1A 125aa B. luti
>SEQ ID NO: 5: GCA_000171035.2_Ena1A 126aa B. cereus
>SEQ ID NO: 6: GCF_007682405.1_Ena1A 126 aa B. tropicus
>SEQ ID NO: 7: GCF_002572325.1_Ena1A 126aa B. wiedmannii
>SEQ ID NO: 8: Bacillus cereus NVH 0075-95 383 Endospore appendage (Ena) 1B amino acid sequence
(GenBank Protein ID: KMP91698.1; 117aa)
>SEQ ID NO: 9: GCF_000161255.1 Ena1B 120aa B. cereus
>SEQ ID NO: 10: GCF_900095655.1_Ena1B 116 aa B. cytotoxicus
>SEQ ID NO: 11: GCA_000171035.2_Ena1B 117 aa B. cereus
>SEQ ID NO: 12: GCF_002572325.1_Ena1B 117 aa B. wiedmannii
>SEQ ID NO: 13: GCF_001884105.1_Ena1B 117 aa B. luti
>SEQ ID NO: 14: GCF_007682405.1_Ena1B 117 aa B. tropicus
>SEQ ID NO: 15: Bacillus cereus NVH 0075-95 383 Endospore appendage (Ena) 1C amino acid sequence
(GenBank Protein ID: KMP91699.1; 155aa)
>SEQ ID NO: 16: GCF_900094915.1_Ena1C 150 aa B. cytotoxicus
>SEQ ID NO: 17: GCF_000789315.1_Ena1C 155 aa B. cereus
>SEQ ID NO: 18: GCF_001044745.1_Ena1C 155 aa B. wiedmannii
>SEQ ID NO: 19: GCF_002568925.1_Ena1C 155 aa B. wiedmannii
>SEQ ID NO: 20: GCF_001884105.1_Ena1C 155 aa B. luti
>SEQ ID NO: 21: Bacillus cytotoxicus NVH 391-98 Endospore appendage (Ena) 2A amino acid sequence
(GenBank Protein ID: ABS21009.1; 126aa)
>SEQ ID NO: 22: GCF_002555305.1_Ena2A 122aa B. wiedmannii
>SEQ ID NO: 23: GCF_000712595.1_Ena2A 119aa B. manliponensis
>SEQ ID NO: 24: GCF_000008005.1_Ena2A 122aa B. cereus
>SEQ ID NO: 25: GCF_000161275.1_Ena2A 122aa B. cereus
>SEQ ID NO: 26: GCF_000007845.1_Ena2A 122 aa B. anthracis
>SEQ ID NO: 27: GCF_002589195.1_Ena2A 122aa B. toyonensis
>SEQ ID NO: 28: GCF_000290695.1_Ena2A 122 aa B. mycoides
>SEQ ID NO: 29: Bacillus cytotoxicus NVH 391-98 Endospore appendage (Ena) 2B amino acid sequence
(GenBank Protein ID: ABS21010.1; 117aa)
>SEQ ID NO: 30: GCF_002555305.1_Ena2B 113 aa B. wiedmannii
>SEQ ID NO: 31: GCF_000712595.1_Ena2B 114aa B. manliponensis
>SEQ ID NO: 32: GCF_000008005.1_Ena2B 112 aa B. cereus
>SEQ ID NO: 33: GCF_000803665.1_Ena2B 110aa B. thuringiensis
>SEQ ID NO: 34: GCF_004023375.1_Ena2B 111 aa B. mycoides
>SEQ ID NO: 35: GCF_000742875.1_Ena2B 114 aa B. anthracis
>SEQ ID NO: 36: GCF_002589605.1_Ena2B 114 aa B. toyonensis
>SEQ ID NO: 37: GCF_900095005.1_Ena2B 114 aa B. mycoides
>SEQ ID NO: 38: Bacillus cytotoxicus NVH 391-98 Endospore appendage (Ena) 2C amino acid sequence
(GenBank Protein ID: ABS21011.1; 150aa)
>SEQ ID NO: 39: GCF_000338755.1_Ena2C 135 B. thuringiensis
>SEQ ID NO: 40: GCF_003386775.1_Ena2C 135 B. mycoides
>SEQ ID NO: 41: GCF_002578975.1 Ena2C 135 B. wiedmannii
>SEQ ID NO: 42: GCF_006349595.1_Ena2C 135 B. pacificus
>SEQ ID NO: 43: GCF_001455345.1_Ena2C 134 B. thuringiensis
>SEQ ID NO: 44: GCF_004023375.1_Ena2C 144 B. mycoides
>SEQ ID NO: 45: GCF_003227955.1_Ena2C 136 B. anthracis
>SEQ ID NO: 46: GCF_001317525.1_Ena2C 136 B. wiedmannii
>SEQ ID NO: 47: GCF_000712595.1_Ena2C 145 B. manliponensis
>SEQ ID NO: 48: GCF_007673655.1_Ena2C 139 B. mycoides
>SEQ ID NO: 49: Bacillus (multispecies-Bacillus cerus ATCC10987-GCF_000008005.1) Endospore
appendage (Ena) 3A amino acid sequence (WP_017562367.1; 133aa)
>SEQ ID NO: 50: WP_157293150.1/1-112 DUF3992 domain-containing protein [Bacillus sp. ms-22]
>SEQ ID NO: 51: WP_105925236.1/1-114 DUF3992 domain-containing protein [Bacillus sp. LLTC93]
>SEQ ID NO: 52: OLP66313.1/1-115 hypothetical protein BACPU_06150 [Bacillus pumilus]
>SEQ ID NO: 53: WP_010787618.1/1-115 DUF3992 domain-containing protein [Bacillus atrophaeus]
>SEQ ID NO: 54: WP_040373377.1/1-116 DUF3992 domain-containing protein
[Peribacillus psychrosaccharolyticus]
>SEQ ID NO: 55: WP_091498261.1/1-115 DUF3992 domain-containing protein [Amphibacillus marinus]
>SEQ ID NO: 56: WP_008633630.1/1-115 multispecies, DUF3992 domain-containing protein
[Bacillaceae]
>SEQ ID NO: 57: WP_124051031.1/1-116 DUF3992 domain-containing protein [Bacillus endophyticus]
>SEQ ID NO: 58: WP_049679853.1/1-114 DUF3992 domain-containing protein
[Peribacillus loiseleuriae]
>SEQ ID NO: 59: WP_062184382.1/1-118 multispecies, DUF3992 domain-containing protein [Bacillales]
>SEQ ID NO: 60: WP_049681018.1/1-118 DUF3992 domain-containing protein [Peribacillus loiseleuriae]
>SEQ ID NO: 61: WP_154975023.1/1-118 DUF3992 domain-containing protein [Bacillus magaterium]
>SEQ ID NO: 62: WP_048022205.1/1-118 DUF3992 domain-containing protein [Bacillus aryabhattai]
>SEQ ID NO: 63: WP_036199318.1/1-114 DUF3992 domain-containing protein
[Lysinibacillus sinduriensis]
>SEQ ID NO: 64: MQR85259.1/1-115 DUF3992 domain-containing protein [Bacillus megaterium]
>SEQ ID NO: 65: WP_111616476.1/1-114 DUF3992 domain-containing protein [Bacillus sp. YR335]
>SEQ ID NO: 66: TDL84647.1/1-113 DUF3992 domain-containing protein [Vibrio vulnificus]
>SEQ ID NO: 67: WP_119116371.1/1-114 DUF3992 domain-containing protein [Peribacillus asahii]
>SEQ ID NO: 68: WP_000057858.1/1-116 DUF3992 domain-containing protein [Bacillus cereus]
>SEQ ID NO: 69: WP_000192611.1/1-114 DUF3992 domain-containing protein [Bacillus cereus]
>SEQ ID NO: 70: WP_000057857.1/1-114 MULTISPECIES: DUF3992 domain-containing protein
[Bacillus cereus group]
>SEQ ID NO: 71: WP_035510401.1/1-114 MULTISPECIES: DUF3992 domain-containing protein
[Halobacillus]
>SEQ ID NO: 72: WP_101934191.1/1-114 DUF3992 domain-containing protein
[Virgibacillus dokdonensis]
>SEQ ID NO: 73: WP_149173096.1/1-114 DUF3992 domain-containing protein [Bacillus sp. BPN334]
>SEQ ID NO: 74: AAS42063.1/1-115 hypothetical protein BCE_3153 [Bacillus cereus ATCC 10987]
>SEQ ID NO: 75: WP_100527630.1/1-114 DUF3992 domain-containing protein [Paenibacillus sp.
GM1FR]
>SEQ ID NO: 76: WP_026691041.1/1-115 DUF3992 domain-containing protein [Bacillus aurantiacus]
>SEQ ID NO: 77: WP_102693317.1/1-113 DUF3992 domain-containing protein
[Rummeliibacillus pycnus]
>SEQ ID NO: 78: WP_071391073.1/1-109 DUF3992 domain-containing protein
[Anaerobacillus alkalidiazotrophicus]
>SEQ ID NO: 79: WP_107839371.1/1-111 DUF3992 domain-containing protein [Lysinibacillus meyeri]
>SEQ ID NO: 80: WP_066166707.1/1-111 DUF3992 domain-containing protein [Metasolibacillus
fluoroglycofenilyticus]
>SEQ ID NO: 81: recombinant Ena1A nucleotide sequence (codes for SEQ ID NO: 82; 429 bp)
>SEQ ID NO: 82: recombinant Ena1A amino acid sequence (with N-terminal 6xHis tag and TEV
cleavage site
MHHHHHHSSGENLYFQGACECSSTVLTCCSDNSSNFVQDKVCNPWSSAEASTFTVYANNVNQNIVGTGYLTYDVGPGVSPANQITVTVLDSGGGTIQ
TFLVNEGTSISFTFRRFNIIQITTPATPIGTYQGEFCITTRYLMA
>SEQ ID NO: 83: recombinant Ena1B nucleotide sequence (codes for SEQ ID NO: 84; 399 bp)
>SEQ ID NO: 84: recombinant Ena1B amino acid sequence (with N-terminal 6xHis tag and TEV
cleavage site)
MHHHHHHSSGENLYFQGNCSTNLSCCANGQKTIVQDKVCIDWTAAATAAIIYADNISQDIYASGYLKVDTGTGPVTIVFYSGGVTGTAVETIVVATG
SSASFTVRRFDTVTILGTAAAETGEFCMTIRYTLS
>SEQ ID NO: 85: recombinant Ena1C nucleotide sequence (codes for SEQ ID NO: 86; 516 bp)
>SEQ ID NO: 86: recombinant Ena1C amino acid sequence (with N-terminal 6xHis tag and TEV
cleavage site)
MHHHHHHSSGENLYFQGKPHKNIGCFAPLSIICQPTCPCPPPILPPERGDAELVTNEFAGDILISNDFIPISQKQLKQTNTTVNIWKNDGIVSLSGT
ISIYNNRNSTNALSIQIISSTTNTFTALPGNTISYTGFDLQSVSVIDIPSDPSIYIEGRYCFQLTYCKSKRDCL
>SEQ ID NO: 87: Ena1B_NM_Oslo (synthetic sequence)
>SEQ ID NO: 88: synthetic peptide in FIG8
>SEQ ID NO: 89: TEV cleavage site

TABLE 3
Oligonucleotide primer sequences.

| Primer | Sequence (5′-3′) | SEQ ID NOS: |
|---|---|---|
| Deletion mutants | | |
| Δena1A | | |
| A: 2184 | AATGGCGCCAGTTCAATTAC | 90 |
| B: 2198 | CCTCTCTACATAGCCTTTCCCCTCTCTCTT | 91 |
| C: 2199 | AAGGCTATGTAGAGAGGGGAATTAGTAT | 92 |
| D: 2178 | CCTCCTATTCTCCCACCTGAAA | 93 |
| Δena1B | | |
| A: 2164 | TCCATGTGGTATGGCAAAAA | 94 |
| B: 2165 | CCATATATTACA [symbol] ACTAATTCCCCTCTC | 95 |
| C: 2166 | AATTAGTATGTAATATATGGTGATTTAAAGATT | 96 |
| D: 2167 | AACCTACTTGCCCCTGTCCT | 97 |
| Δena1C | | |
| A: 2200 | CGCATCTTGTTTAGGTGCAA | 98 |
| B: 2201 | ATTTTTTTGTTATCCTTTTCATAAGACTGTTTAC | 99 |
| C: 2202 | TGAAAAGGATAACAAAAAAATTATTGCTTTTG | 100 |
| D: 2176 | AGGTGGAGGGACAATCCAAAC | 101 |
| Δena1AB | | |
| A: 2164 | TCCATGTGGTATGGCAAAAA | 102 |
| B: 2186 | CCATATATTACATAGCCTTTCCCCTCTC | 103 |
| C: 2197 | AAAGGCTATGTAATATATGGTGATTTAAAGAT | 104 |
| D: 2167 | AACCTACTTGCCCCTGTCCT | 105 |
| RT-PCR | | |
| 2116/2117 | AAGTGCGTCTAATCAACAAGGAAA/GGGAAATCTCCCATGAACACA | 106/107 |
| 2176/2177 | AGGTGGAGGGACAATCCAAAC/GGCGAAACGTAAATGAAATGC | 108/109 |
| 2174/2175 | CCACTGGAAGTAGCGCATCTT/GCCGCTGTTCCAAGAATTGT | 110/111 |
| 2178/1279 | CCTCCTATTCTCCCACCTGAAA/CTCCAGCGAACTCATTGGTAACT | 112/113 |
| 2180/2181 | GGGTGTACGAGGGTGATATGAATT/TGTCGTTCCGCCAAGTGTT | 114/115 |
| Complementation | | |
| 2220/2221 | GCGGATGTTGTTGGACAA/ACGTGCAAACACATGAATCG | 116/117 |

To allow assembly of the PCR fragments, primers B and C contain sequences overlapping each other (italic).
SEQ ID NO:118-139: N-/C-terminal motif consensus sequences
SEQ ID NO: 140: Ena1B-DE-HA insertion variant amino acid sequence (based on SEQ ID NO:8 Ena1B)
SEQ ID NO: 141: Ena1B-DE-Flag insertion variant amino acid sequence (based on SEQ ID NO:8 Ena1B)
SEQ ID NO: 142: Ena1B-HI-HA insertion variant amino acid sequence (based on SEQ ID NO:8 Ena1B)
SEQ ID NO:143: HA-tag
SEQ ID NO:144: FLAG-tag
SEQ ID NO:145: Ena2A amino acid sequence Bacillus thuringiensis (WP_001277540.1)
SEQ ID NO:146: Ena2C amino acid sequence Bacillus thuringiensis (WP_014481960.1)
SEQ ID NOs: 147-150: C-terminal motif consensus sequences.

### REFERENCES

Afonine, P. V., Poon, B. K., Read, R. J., Sobolev, O. V., Terwilliger, T. C., Urzhumtsev, A., and Adams, P. D. (2018). Real-space refinement in PHENIX for cryo-EM and crystallography. Acta Crystallogr D Struct Biol 74, 531-544.
Aluri, S.; Pastuszka, M. K.; Moses, A. S.; MacKay, J. A. Elastin like peptide amphiphiles form nanofibers with tunable length. Biomacromolecules 2012, 13 (9), 2645-54.
Ankolekar, C., and Labbe, R. G. (2010). Physical characteristics of spores of food-associated isolates of the Bacillus cereus group. Appl Environ Microbiol 76, 982-984.
Argimon, S., Abudahab, K., Goater, R. J. E., Fedosejev, A., Bhai, J., Glasner, C., Feil, E. J., Holden, M. T. G., Yeats, C. A., Grundmann, H., et al. (2016). Microreact: visualizing and sharing data for genomic epidemiology and phylogeography. Microb Genom 2, e000093.
Arnaud, M., Chastanet, A., and Debarbouille, M. (2004). New vector for efficient allelic replacement in naturally nontransformable, low-GC-content, gram-positive bacteria. Appl Environ Microbiol 70, 6887-6891.
Atrih, A., and Foster, S. J. (1999). The role of peptidoglycan structure and structural dynamics during endospore dormancy and germination. Antonie Van Leeuwenhoek 75, 299-307.
Bazinet, A. L. (2017). Pan-genome and phylogeny of Bacillus cereus sensu lato. BMC Evol Biol 17, 176.
Bergman, N. H., Anderson, E. C., Swenson, E. E., Niemeyer, M. M., Miyoshi, A. D., and Hanna, P. C. (2006). Transcriptional profiling of the Bacillus anthracis life cycle in vitro and an implied model for regulation of spore formation. J Bacteriol 188, 6092-6100.
Bliven, S., Prlic, A. (2012). Circular permutation in proteins. PLOS Comput. Biol. 8(3):e1002445.
Burnley, T., Palmer, C. M., and Winn, M. (2017). Recent developments in the CCP-EM software suite. Acta Crystallogr D Struct Biol 73, 469-477.
Chen J., and Zou X. Self-assemble peptide biomaterials and their biomedical applications. 2019. Bioactive materials, 4, 120-131.
DesRosier, J. P., and Lara, J. C. (1981). Isolation and properties of pili from spores of Bacillus cereus. J Bacteriol 145, 613-619.
Driks, A. (2007). Surface appendages of bacterial spores. Mol Microbiol 63, 623-625.
Duodu, S., Hoist-Jensen, A., Skjerdal, T., Cappelier, J. M., Pilet, M. F., and Loncarevic, S. (2010). Influence of storage temperature on gene expression and virulence potential of Listeria monocytogenes strains grown in a salmon matrix. Food Microbiol 27, 795-801.
Edgar, R. C. (2004). MUSCLE: a multiple sequence alignment method with reduced time and space complexity. BMC Bioinformatics 5, 113.
Ehling-Schulz, M., Lereclus, D., and Koehler, T. M. (2019). The Bacillus cereus Group: Bacillus Species with Pathogenic Potential. Microbiol Spectr 7.
Emsley, P., Lohkamp, B., Scott, W. G., and Cowtan, K. (2010). Features and development of Coot. Acta crystallographica Section D, Biological crystallography 66, 486-501.
Fallman, E., Schedin, S., Jass, J., Uhlin, B. E., and Axner, 0. (2005). The unfolding of the P pili quaternary structure by stretching is reversible, not plastic. EMBO Rep 6, 52-56.
Farabella, I., Vasishtan, D., Joseph, A. P., Pandurangan, A. P., Sahota, H., and Topf, M. (2015). TEMPy: a Python library for assessment of three-dimensional electron microscopy density fits. J Appl Crystallogr 48, 1314-1323.
Gerhardt, P., and Ribi, E. (1964). Ultrastructure of the Exosporium Enveloping Spores of Bacillus Cereus. Journal of bacteriology 88, 1774-1789.
Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci 27, 14-25.
Gurevich, A., Saveliev, V., Vyahhi, N., and Tesler, G. (2013). QUAST: quality assessment tool for genome assemblies. Bioinformatics 29, 1072-1075.
Hachisuka, Y., and Kuno, T. (1976). Filamentous appendages of Bacillus cereus spores. Jpn J Microbiol 20, 555-558.
He, S., and Scheres, S. H. W. (2017). Helical reconstruction in RELION. J Struct Biol 198, 163-176.
Herrera Estrada, L. P.; Champion, J. A. Protein nanoparticles for therapeutic protein delivery. Biomater. Sci. 2015, 3 (6), 787-99.
Hodgikiss, W. (1971). Filamentous appendages on the spores and exosporium of certain Bacillus species. In Spore research, A. N. Barker, G. W. Gould, and J. Wolf, eds. (London and New York: Academic Press), pp. 211-218.
Jain, A.; Singh, S. K.; Arya, S. K.; Kundu, S. C.; Kapoor, S. Protein Nanoparticles: Promising Platforms for Drug Delivery Applications. ACS Biomater. Sci. Eng. 2018, 4 (12), 3939-3961.
Janes, B. K., and Stibitz, S. (2006). Routine markerless gene replacement in Bacillus anthracis. Infect Immun 74, 1949-1953.
Katoh, K., Rozewicki, J., and Yamada, K. D. (2019). MAFFT online service: multiple sequence alignment, interactive sequence choice and visualization. Brief Bioinform 20, 1160-1166.
Katyal P., Meleties M., and Montclare J. K. Self-assembled Protein- and peptide-based nanomaterials. ACS Biomater. Sci. Eng. 2019, 5, 4132-4147.
Katz, L. S., Griswold, T., Morrison, S. S., Caravas, J. A., Zhang, S., C., d. B. H., Deng, X., and Carleton, A. (2019). Mashtree: a rapid comparison of whole genome sequence
files. Journal of Open Source Software 4.
Kumar, S., Stecher, G., Li, M., Knyaz, C., and Tamura, K. (2018). MEGA X: Molecular Evolutionary Genetics Analysis across Computing Platforms. Mol Biol Evol 35, 1547-1549.
Lindback, T., Mols, M., Basset, C., Granum, P. E., Kuipers, O. P., and Kovacs, A. T. (2012). CodY, a pleiotropic regulator, influences multicellular behaviour and efficient production of virulence factors in Bacillus cereus. Environ Microbiol 14, 2233-2246.
Lombardi, L., Falanga A., Del Genio V., and Galdiero S. A New hope: self-assembling peptides with antimicrobial activity. Pharmaceutics 2019, 11, 166.
Lukaszczyk, M., Pradhan, B., and Remaut, H. (2019). The Biosynthesis and Structures of Bacterial Pili. Subcell Biochem 92, 369-413.
Madslien, E. H., Granum, P. E., Blatny, J. M., and Lindback, T. (2014). L-alanine-induced germination in
Mandlik, A., Swierczynski, A., Das, A., and Ton-That, H. (2008). Pili in Gram-positive bacteria: assembly, involvement in colonization and biofilm development. Trends Microbiol 16, 33-40.
Matsuurua, K. Rational design of self-assembled proteins and peptides for nano- and micro-sized architectures. RSC Adv. 2014, 4(6), 2942-2953.
Melville, S., and Craig, L. (2013). Type IV pili in Gram-positive bacteria. Microbiol Mol Biol Rev 77, 323-341.
Miller, E., Garcia, T., Hultgren, S., and Oberhauser, A. F. (2006). The mechanical properties of E. coli type 1 pili measured by atomic force microscopy techniques. Biophys J 91, 3848-3856.
Mulvey, M. A., Lopez-Boado, Y. S., Wilson, C. L., Roth, R., Parks, W. C., Heuser, J., and Hultgren, S. J. (1998). Induction and evasion of host defenses by type 1-piliated uropathogenic Escherichia coli. Science 282, 1494-1497.
Nei, M., and Gojobori, T. (1986). Simple methods for estimating the numbers of synonymous and nonsynonymous nucleotide substitutions. Mol Biol Evol 3, 418-426.
Ondov, B. D., Treangen, T. J., Melsted, P., Mallonee, A. B., Bergman, N. H., Koren, S., and Phillippy, A. M. (2016). Mash: fast genome and metagenome distance estimation using MinHash. Genome Biol 17, 132.
Page, A. J., Cummins, C. A., Hunt, M., Wong, V. K., Reuter, S., Holden, M. T., Fookes, M., Falush, D., Keane, J. A., and Parkhill, J. (2015). Roary: rapid large-scale prokaryote pan genome analysis. Bioinformatics 31, 3691-3693.
Panessa-Warren, B. J., Tortora, G. T., and Warren, J. B. (2007). High resolution FESEM and TEM reveal bacterial spore attachment. Microsc Microanal 13, 251-266.
Pettersen, E. F., Goddard, T. D., Huang, C. C., Couch, G. S., Greenblatt, D. M., Meng, E. C., and Ferrin, T. E. (2004). UCSF Chimera—a visualization system for exploratory research and analysis. J Comput Chem 25, 1605-1612.
Pfaffl, M. W. (2001). A new mathematical model for relative quantification in real-time RT-PCR. Nucleic Acids Res 29, e45.
Price, M. N., Dehal, P. S., and Arkin, A. P. (2010). FastTree 2—approximately maximum-likelihood trees for large alignments. PLoS One 5, e9490.
Proft, T., and Baker, E. N. (2009). Pili in Gram-negative and Gram-positive bacteria—structure, assembly and their role in disease. Cell Mol Life Sci 66, 613-635.
Remaut, H., and Waksman, G. (2006). Protein-protein interaction through beta-strand addition. Trends Biochem Sci 31, 436-444.
Richardson, J. S. (1981). The anatomy and taxonomy of protein structure. Adv Protein Chem 34, 167-339.
Rode, L. J., Pope, L., Filip, C., and Smith, L. D. (1971). Spore appendages and taxonomy of Clostridium sordellii. Journal of bacteriology 108, 1384-1389.
Rohou, A., and Grigorieff, N. (2015). CTFFIND4: Fast and accurate defocus estimation from electron micrographs. J Struct Biol 192, 216-221.
Sauer, F. G., Futterer, K., Pinkner, J. S., Dodson, K. W., Hultgren, S. J., and Waksman, G. (1999). Structural basis of chaperone function and pilus biogenesis. Science 285, 1058-1061.
Seemann, T. (2014). Prokka: rapid prokaryotic genome annotation. Bioinformatics 30, 2068-2069.
Setlow, P. (2014). Germination of spores of Bacillus species: what we know and do not know. Journal of bacteriology 196, 1297-1305.
Smirnova, T. A., Zubasheva, M. V., Shevliagina, N. V., Nikolaenko, M. A., and Azizbekian, R. R. (2013). [Electron microscopy of the surfaces of bacillary spores]. Mikrobiologiia 82, 698-706.
Stewart, G. C. (2015). The Exosporium Layer of Bacterial Spores: a Connection to the Environment and the Infected Host. Microbiol Mol Biol Rev 79, 437-457.
Tamura, K., Nei, M., and Kumar, S. (2004). Prospects for inferring very large phylogenies by using the neighbor-joining method. Proc Natl Acad Sci USA 101, 11030-11035.
Todd, S. J., Moir, A. J., Johnson, M. J., & Moir, A. (2003). Genes of Bacillus cereus and Bacillus anthracis encoding proteins of the exosporium. Journal of bacteriology, 185(11), 3373-3378.
Ton-That, H., and Schneewind, O. (2004). Assembly of pili in Gram-positive bacteria. Trends Microbiol 12, 228-234.
Walker, J. R., Gnanam, A. J., Blinkova, A. L., Hermandson, M. J., Karymov, M. A., Lyubchenko, Y. L., Graves, P. R., Haystead, T. A., and Linse, K. D. (2007). Clostridium taeniosporum spore ribbon-like appendage structure, composition and genes. Mol Microbiol 63, 629-643.
Wang, J., Mei, H., Zheng, C., Qian, H., Cui, C., Fu, Y., Su, J., Liu, Z., Yu, Z., and He, J. (2013). The metabolic regulation of sporulation and parasporal crystal formation in Bacillus thuringiensis revealed by transcriptomics and proteomics. Mol Cell Proteomics 12, 1363-1376.
Wheeler, T. J., Clements, J. & Finn, R. D. Skylign: a tool for creating informative, interactive logos representing sequence alignments and profile hidden Markov models. BMC Bioinformatics 15, 7 (2014). https://doi.org/10.1186/1471-2105-15-7
Xu, Q., Shoji, M., Shibata, S., Naito, M., Sato, K., Elsliger, M. A., Grant, J. C., Axelrod, H. L., Chiu, H. J., Farr, C. L., et al. (2016). A Distinct Type of Pilus from the Human Microbiome. Cell 165, 690-703.
Yu, Y.-C.; Berndt, P.; Tirrell, M.; Fields, G. B. Self-Assembling Amphiphiles for Construction of Protein Molecular Architecture. J. Am. Chem. Soc. 1996, 118 (50), 12515-12520.
Zheng, S. Q., Palovcak, E., Armache, J. P., Verba, K. A., Cheng, Y., and Agard, D. A. (2017). MotionCor2: anisotropic correction of beam-induced motion for improved cryo-electron microscopy. Nat Methods 14, 331-332.
Zivanov, J., Nakane, T., Forsberg, B. O., Kimanius, D., Hagen, W. J., Lindahl, E., and Scheres, S. H. (2018). New tools for automated high-resolution cryo-EM structure determination in RELION-3. Elife 7.
Zuckerkandl, E., and Pauling, L. (1965). Molecules as documents of evolutionary history. J Theor Biol 8, 357-366.
The Pfam protein families database in 2019: S. El-Gebali, J. Mistry, A. Bateman, S. R. Eddy, A. Luciani, S. C. Potter, M. Qureshi, L. J. Richardson, G. A. Salazar, A. Smart, E. L. L. Sonnhammer, L. Hirsh, L. Paladin, D. Piovesan, S. C. E. Tosatto, R. D. Finn. Nucleic Acids Research (2019) doi: 10.1093/nar/gky995.

1. A multimer of a self-assembling protein, wherein:
the multimer comprises at least seven subunits of the self-assembling protein;
the self-assembling proteins are present as non-covalently linked subunits of the multimer; and
the self-assembling protein, comprises a DUF3992 domain, and wherein the self-assembling protein has a three-dimensional predicted fold matching the Ena1B structure with a fold similarity Z-score of 6.5 or more, wherein Ena1B corresponds to SEQ ID NO:8.

2. The multimer of claim 1, wherein the self-assembling protein comprises an amino acid sequence selected from the group consisting of SEQ ID NOs: 1-80, 145, 146, and a homologue with at least 80% identity of any one thereof.

3. The multimer of claim 1, wherein the self-assembling protein is an engineered self-assembling protein.

4. The multimer of claim 1, wherein at least one of the self-assembling proteins comprises a sequence heterologous to the DUF3992 domain.

5. The multimer of claim 1, wherein at least one self-assembling protein of the multimer is an engineered self-assembling protein.

6. The multimer of claim 4, wherein at least one self-assembling protein subunit of the multimer comprises an N-terminal region which comprises the amino acid sequence motif ZXₙCCXₘC, wherein Z is Leu, Ile, Val, or Phe, n is 1 or 2, and m is between 10 and 12, and comprise a C-terminal region which comprises the amino acid sequence motif GX_{2/3}CX₄Y, and wherein X is any amino acid.

7. The multimer of claim 6, wherein at least one self-assembling protein subunit of the multimer comprises an amino acid sequence motif ZXₙCCXₘC, wherein m is between 13 and 16, or wherein m is 7-9.

8. The multimer of claim 4, wherein the self-assembling protein subunits of the multimer comprise an N-terminal region which comprises the amino acid sequence motif ZXₙC(C)XₘC, wherein Z is Leu, Ile, Val, or Phe, n is 1 or 2, and m is between 10 and 12, (C) is an optional Cys, and comprise a C-terminal region which comprises the amino acid sequence motif S-Z-N-Y-X-B, wherein Z is Leu or Ile, B is Phe or Tyr, and X is any amino acid.

9. The multimer of claim 1, wherein the multimer is comprised in a protein fiber comprising at least two multimers of claim 4, wherein the multimers are longitudinally stacked and covalently linked through at least one disulphide bond.

10. The multimer of claim 9, wherein the self-assembling protein subunits of the multimers are identical.

11. The multimer of claim 9, wherein the protein fiber is an engineered protein fiber characterized in that the multimers comprise at least one engineered multimer or engineered self-assembling protein.

12. A chimeric gene comprising the following operably linked DNA elements: a) a heterologous promoter, and b) a nucleic acid sequence encoding a self-assembling protein comprising a DUF3992 domain, and wherein the protein has a three-dimensional predicted fold matching the Ena1B structure with a fold similarity Z-score of 6.5 or more, wherein Ena1B corresponds to SEQ ID NO:8.

13. The chimeric gene of claim 12, wherein the chimeric gene is comprised in a host.

14. The chimeric gene of claim 12, wherein the chimeric gene is comprised in a bacterial endospore.

15. The multimer of claim 1, wherein the multimer is comprised in/on a modified surface.

16. A method of producing a self-assembling protein, the method comprising:
a. expressing a chimeric gene encoding the self-assembling protein in a cell, wherein
the self-assembling protein comprises a DUF3992 domain, and wherein the self-assembling protein has a three-dimensional predicted fold matching the Ena1B structure with a fold similarity Z-score of 6.5 or more, wherein Ena1B corresponds to SEQ ID NO:8;
the chimeric gene comprises a nucleic acid encoding the self-assembling protein operatively linked to a heterologous promoter; and
the nucleic acid sequence encoding the self-assembling protein optionally comprises a heterologous N- or C-terminal tag, and, and/or
b. isolating monomers and/or multimers of the self-assembling protein from the cell.

17. The method according to claim 16, wherein the heterologous N- or C-terminal tag comprises at least 6 amino acid residues.

18. The method according to claim 16, wherein the heterologous N- or C-terminal tag is a removable tag, and wherein the method further comprises removing the tag from the protein subunits to allow fiber formation.

19. A method of producing the multimer of claim 9 in a host cell, the method comprising:
expressing a chimeric gene encoding the self-assembling peptide in the host cell, wherein the self-assembling protein has no heterologous tag which allows fiber formation in cellulo, and/or
isolating multimers and/or fibers comprising the self-assembling protein from the host cell.

20. The multimer of claim 15, wherein the surface has been modified by the covalent binding of the multimer to the surface.

21. The method according to claim 19, wherein the isolating multimers and/or fibers comprising the self-assembling protein from the host cell comprises cell lysis.

22. The multimer of claim 1, wherein the multimer is comprised in a thin protein film or a hydrogel.
